SEQUENCE LISTING (280 sequences)

SEQ ID NO: 1 - protein, 227 aa, Homo sapiens
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 2 - protein, 27 aa, Synthetic construct
  VARIANT 1: note = F or Y
  VARIANT 2: note = F, I, L or V
  VARIANT 3: note = A or Q
  VARIANT 6: note = A, F, H, Y or W
  VARIANT 9: note = F or Y
  VARIANT 11: note = A or L
  VARIANT 13: note = C, H, N, Q, W or Y
  VARIANT 15: note = D, N, Q, S or T
  VARIANT 17: note = A , D, E, G, H, L, N, Q, S, T or Y
  VARIANT 10: note = N, Q or T
  VARIANT 25: note = A, Q or S
XXXAWXEIXXXPXLXDXQWPAFIEXLH

SEQ ID NO: 3 - protein, 58 aa, Synthetic construct
  VARIANT 1: note = G, I, L, N or V
  VARIANT 2: note = D, E or Q
  VARIANT 3: note = D, E, G, N or Q
  VARIANT 4: note = D, E, H, N, R or T
  VARIANT 5: note = E, I, L, T or V
  VARIANT 6: note = F, W or Y
  VARIANT 7: note = A, G, H, I, L, N, P, Q, R, T or V
  VARIANT 8: note = A, L or E
  VARIANT 9: note = F or Y
  VARIANT 10: note = F, I, L or V
  VARIANT 11: note = A or Q
  VARIANT 14: note = A, F, H, Y or W
  VARIANT 17: note = F or Y
  VARIANT 18: note = N, Q or T
  VARIANT 19: note = A or L
  VARIANT 21: note = C, H, N, Q, W or Y
  VARIANT 23: note = D, N, Q, S or T
  VARIANT 25: note = A, D, E, G, H, L, N, Q, S, T or Y
  VARIANT 33: note = A, Q or S
  VARIANT 36: note = A, D or E
  VARIANT 37: note = D or E
  VARIANT 41: note = A, R or W
  VARIANT 42: note = A, D or T
  VARIANT 46: note = A, E or K
  VARIANT 50: note = Q or R
  VARIANT 52: note = N or E
  VARIANT 53: note = D , E, K or T
  VARIANT 54: note = A or I
  VARIANT 56: note = A or C
  VARIANT 57: note = K or P
  VARIANT 58: note = K or E
  VARIANT 43: note = E, N, Q or S
XXXXXXXXXXXAWXEIXXXPXLXDXQWPAFIEXLHXXPSQXXXLLXEARXLXXXQXXX

SEQ ID NO: 4 - protein, 155 aa, Synthetic construct
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTR

SEQ ID NO: 5 - protein, 233 aa, Synthetic construct
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIHHHHHH

SEQ ID NO: 6 - protein, 279 aa, Synthetic construct
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGAAKAAFLPGMNPPPYSQFLSRRNEIPLIHFNTPIPRQHTQSAEDDSERDPLNVLKPRARMTPAPASCSQELPSAEDNSPMASDPLGVVRGGRVNTHAGGTGPEGCRPFAKFIGGGGSGGGGSGGGGSGLNDIFEAQKIEWHEGGGGSGGGGSEDQVDPRLIDGK

SEQ ID NO: 7 - protein, 447 aa, Synthetic construct
QVQLVQSGAEVKKPGASVKVSCKASGYTFTNHYMHWVRQAPGQGLEWMGIINPISGSTSNAQKFQGRVTMTRDTSTSTVYMELSSLRSEDTAVYYCARDIVDAFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Synthetic construct
AIQLTQSPSSLSASVGDRVTITCRASQGISSALVWYQQKPGKAPKLLIYDASSLESGVPSRFSGSGSGTDFTLTISSLQPEDFATYYCQQFNDYFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 20 aa, Synthetic construct
QRLMEDICLPRWGCLWEDDF

SEQ ID NO: 10 - protein, 58 aa, Synthetic construct
VDNREFNAYLQAWYEIFNLPNLNDNQWPAFIESLHEDPSQWANLLAEARRLNDAQAPE

SEQ ID NO: 11 - protein, 27 aa, Synthetic construct
YLQAWYEIFNLPNLNDNQWPAFIESLH

SEQ ID NO: 12 - protein, 27 aa, Synthetic construct
YLQAWYEIFNLPWLNDNQWPAFIESLH

SEQ ID NO: 13 - protein, 27 aa, Synthetic construct
YLQAWYEIFNLPYLNDNQWPAFIESLH

SEQ ID NO: 14 - protein, 27 aa, Synthetic construct
YLQAWYEIFQLPNLNDNQWPAFIESLH

SEQ ID NO: 15 - protein, 27 aa, Synthetic construct
YLQAWWEIFNLPNLNDNQWPAFIESLH

SEQ ID NO: 16 - protein, 27 aa, Synthetic construct
YLQAWFEIFNLPNLNDNQWPAFIESLH

SEQ ID NO: 17 - protein, 27 aa, Synthetic construct
YLQAWHEIFNLPNLNDNQWPAFIESLH

SEQ ID NO: 18 - protein, 27 aa, Synthetic construct
YLAAWYEIFNLPNLNDNQWPAFIESLH

SEQ ID NO: 19 - protein, 27 aa, Synthetic construct
YLQAWYEIFNLPHLNDNQWPAFIESLH

SEQ ID NO: 20 - protein, 27 aa, Synthetic construct
YLQAWYEIFNLPNLNDNQWPAFIEALH

SEQ ID NO: 21 - protein, 27 aa, Synthetic construct
YLQAWYEIFNLPNLNDNQWPAFIEQLH

SEQ ID NO: 22 - protein, 27 aa, Synthetic construct
YLQAWYEIFNLPNLQDNQWPAFIESLH

SEQ ID NO: 23 - protein, 27 aa, Synthetic construct
YLQAWYEIFNLPNLNDQQWPAFIESLH

SEQ ID NO: 24 - protein, 27 aa, Synthetic construct
YLQAWYEIFNLPNLNDDQWPAFIESLH

SEQ ID NO: 25 - protein, 27 aa, Synthetic construct
YLQAWYEIFNLPNLNDEQWPAFIESLH

SEQ ID NO: 26 - protein, 27 aa, Synthetic construct
YLQAWYEIFNLPNLNDSQWPAFIESLH

SEQ ID NO: 27 - protein, 27 aa, Synthetic construct
YLQAWYEIFNLPNLNDTQWPAFIESLH

SEQ ID NO: 28 - protein, 27 aa, Synthetic construct
YLQAWYEIFNLPNLNDHQWPAFIESLH

SEQ ID NO: 29 - protein, 27 aa, Synthetic construct
YLQAWYEIFNLPNLNDAQWPAFIESLH

SEQ ID NO: 30 - protein, 27 aa, Synthetic construct
YLQAWYEIFNLPNLNDLQWPAFIESLH

SEQ ID NO: 31 - protein, 27 aa, Synthetic construct
YLQAWYEIFNLPNLTDNQWPAFIESLH

SEQ ID NO: 32 - protein, 27 aa, Synthetic construct
YLQAWYEIFNLPNLTDEQWPAFIESLH

SEQ ID NO: 33 - protein, 27 aa, Synthetic construct
YLQAWYEIFNLPNLTDQQWPAFIESLH

SEQ ID NO: 34 - protein, 27 aa, Synthetic construct
YLQAWYEIFNLPNLSDNQWPAFIESLH

SEQ ID NO: 35 - protein, 27 aa, Synthetic construct
YLQAWYEIFNLPNLNDGQWPAFIESLH

SEQ ID NO: 36 - protein, 27 aa, Synthetic construct
YVQAWYEIFNLPNLNDNQWPAFIESLH

SEQ ID NO: 37 - protein, 27 aa, Synthetic construct
YIQAWYEIFNLPNLNDNQWPAFIESLH

SEQ ID NO: 38 - protein, 27 aa, Synthetic construct
YFQAWYEIFNLPNLNDNQWPAFIESLH

SEQ ID NO: 39 - protein, 27 aa, Synthetic construct
YLQAWYEIFNLPQLNDNQWPAFIESLH

SEQ ID NO: 40 - protein, 27 aa, Synthetic construct
YLQAWYEIFNLPNLQDQQWPAFIESLH

SEQ ID NO: 41 - protein, 27 aa, Synthetic construct
YLQAWYEIFNLPHLNDQQWPAFIESLH

SEQ ID NO: 42 - protein, 27 aa, Synthetic construct
YLQAWYEIFNLPNLDDAQWPAFIESLH

SEQ ID NO: 43 - protein, 27 aa, Synthetic construct
YLQAWYEIFNLPNLTDAQWPAFIESLH

SEQ ID NO: 44 - protein, 27 aa, Synthetic construct
YLQAWYEIFNLPNLDDQQWPAFIESLH

SEQ ID NO: 45 - protein, 27 aa, Synthetic construct
YLQAWYEIFNLPHLNDAQWPAFIESLH

SEQ ID NO: 46 - protein, 27 aa, Synthetic construct
YLQAWYEIFNLPNLNDYQWPAFIESLH

SEQ ID NO: 47 - protein, 27 aa, Synthetic construct
YLQAWYEIFNLPHLTDQQWPAFIESLH

SEQ ID NO: 48 - protein, 27 aa, Synthetic construct
YLQAWYEIFNLPNLTDYQWPAFIESLH

SEQ ID NO: 49 - protein, 27 aa, Synthetic construct
YLQAWYEIFTLPHLTDQQWPAFIESLH

SEQ ID NO: 50 - protein, 27 aa, Synthetic construct
YLQAWYEIFNLPCLNDNQWPAFIESLH

SEQ ID NO: 51 - protein, 27 aa, Synthetic construct
YLQAWYEIFNLPCLNDQQWPAFIESLH

SEQ ID NO: 52 - protein, 27 aa, Synthetic construct
YLQAWYEIFNLPCLTDSQWPAFIESLH

SEQ ID NO: 53 - protein, 58 aa, Synthetic construct
VDNREFNAYLQAWYEIFNLPNLNDNQWPAFIESLHEDPSQWANLLAEARRLNDAQAKE

SEQ ID NO: 54 - protein, 58 aa, Synthetic construct
VDNTLYPLYLQAWYEIFNLPNLNDNQWPAFIESLHEDPSQWANLLAEARRLNKAQAPE

SEQ ID NO: 55 - protein, 58 aa, Synthetic construct
VDNREFNAYLQAWYEIFNLPNLNDNQWPAFIESLHEDPSQWANLLAEARRLNKAQAPE

SEQ ID NO: 56 - protein, 58 aa, Synthetic construct
VDNTLYPLYLQAWYEIFNLPNLNDNQWPAFIESLHEDPSQWANLLKEARRLNDAQAPE

SEQ ID NO: 57 - protein, 58 aa, Synthetic construct
VDNREFNAYLQAWYEIFNLPNLNDNQWPAFIESLHEDPSQWANLLKEARRLNDAQAPE

SEQ ID NO: 58 - protein, 58 aa, Synthetic construct
LDNDEFQLYLQAWYEIFNLPNLNDNQWPAFIESLHDDPSQWANLLEEARRLNDAQAPK

SEQ ID NO: 59 - protein, 58 aa, Synthetic construct
LDNDEFTLYLQAWYEIFNLPNLNDNQWPAFIESLHDDPSQWANLLEEARRLNDAQAPK

SEQ ID NO: 60 - protein, 58 aa, Synthetic construct
LDNTEFILYLQAWYEIFNLPNLNDNQWPAFIESLHDDPSQWANLLEEARRLNDAQAPK

SEQ ID NO: 61 - protein, 58 aa, Synthetic construct
LDNDTFILYLQAWYEIFNLPNLNDNQWPAFIESLHDDPSQWANLLEEARRLNDAQAPK

SEQ ID NO: 62 - protein, 58 aa, Synthetic construct
LDNDEFILYLQAWYEIFNLPNLNDNQWPAFIESLHADPSQWANLLEEARRLNDAQAPK

SEQ ID NO: 63 - protein, 58 aa, Synthetic construct
VDNDEFILYLQAWYEIFNLPNLNDNQWPAFIESLHADPSQWANLLAEARRLNDAQAPK

SEQ ID NO: 64 - protein, 58 aa, Synthetic construct
LDNDEFILYLQAWYEIFNLPNLNDNQWPAFIESLHADPSQWANLLAEARRLNDAQAPK

SEQ ID NO: 65 - protein, 58 aa, Synthetic construct
IDNDEFILYLQAWYEIFNLPNLNDNQWPAFIESLHDDPSQWANLLAEARRLNDAQAPK

SEQ ID NO: 66 - protein, 58 aa, Synthetic construct
NDNDEFILYLQAWYEIFNLPNLNDNQWPAFIESLHDDPSQWANLLAEARRLNDAQAPK

SEQ ID NO: 67 - protein, 58 aa, Synthetic construct
VDQDEFILYLQAWYEIFNLPNLNDNQWPAFIESLHDDPSQWANLLAEARRLNDAQAPK

SEQ ID NO: 68 - protein, 58 aa, Synthetic construct
VDNTEFILYLQAWYEIFNLPNLNDNQWPAFIESLHDDPSQWANLLAEARRLNDAQAPK

SEQ ID NO: 69 - protein, 58 aa, Synthetic construct
VDNNEFILYLQAWYEIFNLPNLNDNQWPAFIESLHDDPSQWANLLAEARRLNDAQAPK

SEQ ID NO: 70 - protein, 58 aa, Synthetic construct
VDNHEFILYLQAWYEIFNLPNLNDNQWPAFIESLHDDPSQWANLLAEARRLNDAQAPK

SEQ ID NO: 71 - protein, 58 aa, Synthetic construct
VDNREFILYLQAWYEIFNLPNLNDNQWPAFIESLHDDPSQWANLLAEARRLNDAQAPK

SEQ ID NO: 72 - protein, 58 aa, Synthetic construct
VDNDTFILYLQAWYEIFNLPNLNDNQWPAFIESLHDDPSQWANLLAEARRLNDAQAPK

SEQ ID NO: 73 - protein, 58 aa, Synthetic construct
VDNDVFILYLQAWYEIFNLPNLNDNQWPAFIESLHDDPSQWANLLAEARRLNDAQAPK

SEQ ID NO: 74 - protein, 58 aa, Synthetic construct
VDNDEFQLYLQAWYEIFNLPNLNDNQWPAFIESLHDDPSQWANLLAEARRLNDAQAPK

SEQ ID NO: 75 - protein, 58 aa, Synthetic construct
VDNDEFNLYLQAWYEIFNLPNLNDNQWPAFIESLHDDPSQWANLLAEARRLNDAQAPK

SEQ ID NO: 76 - protein, 58 aa, Synthetic construct
VDNDEFLLYLQAWYEIFNLPNLNDNQWPAFIESLHDDPSQWANLLAEARRLNDAQAPK

SEQ ID NO: 77 - protein, 58 aa, Synthetic construct
VDNDEFALYLQAWYEIFNLPNLNDNQWPAFIESLHDDPSQWANLLAEARRLNDAQAPK

SEQ ID NO: 78 - protein, 58 aa, Synthetic construct
VDNDEFRLYLQAWYEIFNLPNLNDNQWPAFIESLHDDPSQWANLLAEARRLNDAQAPK

SEQ ID NO: 79 - protein, 58 aa, Synthetic construct
VDNDEFGLYLQAWYEIFNLPNLNDNQWPAFIESLHDDPSQWANLLAEARRLNDAQAPK

SEQ ID NO: 80 - protein, 58 aa, Synthetic construct
VDNDEFPLYLQAWYEIFNLPNLNDNQWPAFIESLHDDPSQWANLLAEARRLNDAQAPK

SEQ ID NO: 81 - protein, 58 aa, Synthetic construct
VDNDEFHLYLQAWYEIFNLPNLNDNQWPAFIESLHDDPSQWANLLAEARRLNDAQAPK

SEQ ID NO: 82 - protein, 58 aa, Synthetic construct
VDNDEFVLYLQAWYEIFNLPNLNDNQWPAFIESLHDDPSQWANLLAEARRLNDAQAPK

SEQ ID NO: 83 - protein, 58 aa, Synthetic construct
LDNDEFILYLQAWYEIFNLPWLNDNQWPAFIESLHADPSQWANLLEEARRLNDAQAPK

SEQ ID NO: 84 - protein, 58 aa, Synthetic construct
LDNDEFILYLQAWYEIFNLPYLNDNQWPAFIESLHADPSQWANLLEEARRLNDAQAPK

SEQ ID NO: 85 - protein, 58 aa, Synthetic construct
LDNDEFIEYLQAWYEIFNLPNLNDNQWPAFIESLHDDPSQRANLLAEARRLNDAQAPK

SEQ ID NO: 86 - protein, 58 aa, Synthetic construct
LDNDEFILYLQAWYEIFQLPNLNDNQWPAFIESLHDDPSQWAQLLEEARRLNDAQAPK

SEQ ID NO: 87 - protein, 58 aa, Synthetic construct
LDNDEFILYLQAWYEIFNLPWLNDNQWPAFIESLHDDPSQWAQLLEEARRLNDAQAPK

SEQ ID NO: 88 - protein, 58 aa, Synthetic construct
LDNDEFILYLQAWYEIFNLPWLNDNQWPAFIESLHADPSQWAQLLEEARRLNDAQAPK

SEQ ID NO: 89 - protein, 58 aa, Synthetic construct
VEGDEFNAYLQAWYEIFNLPNLNDNQWPAFIESLHEDPSQWANLLAEARRLNDAQAPK

SEQ ID NO: 90 - protein, 58 aa, Synthetic construct
VEGEEFNAYLQAWYEIFNLPNLNDNQWPAFIESLHEDPSQWANLLAEARRLNDAQAPK

SEQ ID NO: 91 - protein, 58 aa, Synthetic construct
VDNTIFNAYLQAWYEIFNLPNLNDNQWPAFIESLHEDPSQWANLLAEARRLNDAQAPK

SEQ ID NO: 92 - protein, 58 aa, Synthetic construct
VDNTVFNAYLQAWYEIFNLPNLNDNQWPAFIESLHEDPSQWANLLAEARRLNDAQAPK

SEQ ID NO: 93 - protein, 58 aa, Synthetic construct
VDNDLFNAYLQAWYEIFNLPNLNDNQWPAFIESLHEDPSQWANLLAEARRLNDAQAPK

SEQ ID NO: 94 - protein, 58 aa, Synthetic construct
VDNDIFNAYLQAWYEIFNLPNLNDNQWPAFIESLHEDPSQWANLLAEARRLNDAQAPK

SEQ ID NO: 95 - protein, 58 aa, Synthetic construct
VDNDVFNAYLQAWYEIFNLPNLNDNQWPAFIESLHEDPSQWANLLAEARRLNDAQAPK

SEQ ID NO: 96 - protein, 58 aa, Synthetic construct
VENTLFNAYLQAWYEIFNLPNLNDNQWPAFIESLHEDPSQWANLLAEARRLNDAQAPK

SEQ ID NO: 97 - protein, 58 aa, Synthetic construct
VENTIFNAYLQAWYEIFNLPNLNDNQWPAFIESLHEDPSQWANLLAEARRLNDAQAPK

SEQ ID NO: 98 - protein, 58 aa, Synthetic construct
VENTVFNAYLQAWYEIFNLPNLNDNQWPAFIESLHEDPSQWANLLAEARRLNDAQAPK

SEQ ID NO: 99 - protein, 58 aa, Synthetic construct
VDNTLFNAYLQAWYEIFNLPNLNDNQWPAFIESLHEDPSQWANLLAEARRLNDAQAPK

SEQ ID NO: 100 - protein, 58 aa, Synthetic construct
VDNTLFNAYLQAWWEIFNLPNLNDNQWPAFIESLHEDPSQWANLLAEARRLNDAQAPK

SEQ ID NO: 101 - protein, 58 aa, Synthetic construct
VDNTLFNAYLQAWFEIFNLPNLNDNQWPAFIESLHEDPSQWANLLAEARRLNDAQAPK

SEQ ID NO: 102 - protein, 58 aa, Synthetic construct
VDNTLFNAYLQAWHEIFNLPNLNDNQWPAFIESLHEDPSQWANLLAEARRLNDAQAPK

SEQ ID NO: 103 - protein, 58 aa, Synthetic construct
VDNTEFILYLQAWWEIFNLPNLNDNQWPAFIESLHDDPSQWANLLAEARRLNDAQAPK

SEQ ID NO: 104 - protein, 58 aa, Synthetic construct
VDNTEFILYLQAWFEIFNLPNLNDNQWPAFIESLHDDPSQWANLLAEARRLNDAQAPK

SEQ ID NO: 105 - protein, 58 aa, Synthetic construct
VDNTEFILYLQAWHEIFNLPNLNDNQWPAFIESLHDDPSQWANLLAEARRLNDAQAPK

SEQ ID NO: 106 - protein, 58 aa, Synthetic construct
VDNTLFILYLQAWWEIFNLPNLNDNQWPAFIESLHDDPSQWANLLAEARRLNDAQAPK

SEQ ID NO: 107 - protein, 58 aa, Synthetic construct
VEGDEFNAYLQAWYEIFNLPNLNDNQWPAFIESLHEDPSQWANLLEEARRLNDAQAPK

SEQ ID NO: 108 - protein, 58 aa, Synthetic construct
VEGEEFNAYLQAWYEIFNLPNLNDNQWPAFIESLHEDPSQWANLLEEARRLNDAQAPK

SEQ ID NO: 109 - protein, 58 aa, Synthetic construct
VDNTLFNAYLQAWYEIFNLPNLNDNQWPAFIESLHEDPSQWANLLEEARRLNDAQAPK

SEQ ID NO: 110 - protein, 58 aa, Synthetic construct
VDNTIFNAYLQAWYEIFNLPNLNDNQWPAFIESLHEDPSQWANLLEEARRLNDAQAPK

SEQ ID NO: 111 - protein, 58 aa, Synthetic construct
VDNTVFNAYLQAWYEIFNLPNLNDNQWPAFIESLHEDPSQWANLLEEARRLNDAQAPK

SEQ ID NO: 112 - protein, 58 aa, Synthetic construct
VDNDLFNAYLQAWYEIFNLPNLNDNQWPAFIESLHEDPSQWANLLEEARRLNDAQAPK

SEQ ID NO: 113 - protein, 58 aa, Synthetic construct
VDNDIFNAYLQAWYEIFNLPNLNDNQWPAFIESLHEDPSQWANLLEEARRLNDAQAPK

SEQ ID NO: 114 - protein, 58 aa, Synthetic construct
VDNDVFNAYLQAWYEIFNLPNLNDNQWPAFIESLHEDPSQWANLLEEARRLNDAQAPK

SEQ ID NO: 115 - protein, 58 aa, Synthetic construct
VENTLFNAYLQAWYEIFNLPNLNDNQWPAFIESLHEDPSQWANLLEEARRLNDAQAPK

SEQ ID NO: 116 - protein, 58 aa, Synthetic construct
VENTIFNAYLQAWYEIFNLPNLNDNQWPAFIESLHEDPSQWANLLEEARRLNDAQAPK

SEQ ID NO: 117 - protein, 58 aa, Synthetic construct
VDNTLFNAYLQAWWEIFNLPNLNDNQWPAFIESLHEDPSQWANLLEEARRLNDAQAPK

SEQ ID NO: 118 - protein, 58 aa, Synthetic construct
VDNTLFNAYLQAWHEIFNLPNLNDNQWPAFIESLHEDPSQWANLLEEARRLNDAQAPK

SEQ ID NO: 119 - protein, 58 aa, Synthetic construct
VDNTEFILYLQAWWEIFNLPNLNDNQWPAFIESLHDDPSQWANLLEEARRLNDAQAPK

SEQ ID NO: 120 - protein, 58 aa, Synthetic construct
LDNEEFNAYLQAWYEIFNLPNLNDNQWPAFIESLHEDPSQWANLLAEARRLNDAQAPK

SEQ ID NO: 121 - protein, 58 aa, Synthetic construct
LDNREFQAYLQAWYEIFNLPNLNDNQWPAFIESLHEDPSQWANLLAEARRLNDAQAPK

SEQ ID NO: 122 - protein, 58 aa, Synthetic construct
VDQEEFNAYLQAWYEIFNLPNLNDNQWPAFIESLHEDPSQWANLLAEARRLNDAQAPK

SEQ ID NO: 123 - protein, 58 aa, Synthetic construct
VDEEEFNAYLQAWYEIFNLPNLNDNQWPAFIESLHEDPSQWANLLAEARRLNDAQAPK

SEQ ID NO: 124 - protein, 58 aa, Synthetic construct
VDNEEFQAYLQAWYEIFNLPNLNDNQWPAFIESLHEDPSQWANLLAEARRLNDAQAPK

SEQ ID NO: 125 - protein, 58 aa, Synthetic construct
LDNELFNAYLQAWYEIFNLPNLNDNQWPAFIESLHEDPSQWANLLAEARRLNDAQAPK

SEQ ID NO: 126 - protein, 58 aa, Synthetic construct
LDQTEFNAYLQAWYEIFNLPNLNDNQWPAFIESLHEDPSQWANLLAEARRLNDAQAPK

SEQ ID NO: 127 - protein, 58 aa, Synthetic construct
LDETEFNAYLQAWYEIFNLPNLNDNQWPAFIESLHEDPSQWANLLAEARRLNDAQAPK

SEQ ID NO: 128 - protein, 58 aa, Synthetic construct
LDDEEFNAYLQAWYEIFNLPNLNDNQWPAFIESLHEDPSQWANLLAEARRLNDAQAPK

SEQ ID NO: 129 - protein, 58 aa, Synthetic construct
LDNEEFQAYLQAWYEIFNLPNLNDNQWPAFIESLHEDPSQWANLLAEARRLNDAQAPK

SEQ ID NO: 130 - protein, 58 aa, Synthetic construct
VDETLFNAYLQAWYEIFNLPNLNDNQWPAFIESLHEDPSQWANLLAEARRLNDAQAPK

SEQ ID NO: 131 - protein, 58 aa, Synthetic construct
VDQTEFQAYLQAWYEIFNLPNLNDNQWPAFIESLHEDPSQWANLLAEARRLNDAQAPK

SEQ ID NO: 132 - protein, 58 aa, Synthetic construct
VDQEEFQAYLQAWYEIFNLPNLNDNQWPAFIESLHEDPSQWANLLAEARRLNDAQAPK

SEQ ID NO: 133 - protein, 58 aa, Synthetic construct
VDEEEFQAYLQAWYEIFNLPNLNDNQWPAFIESLHEDPSQWANLLAEARRLNDAQAPK

SEQ ID NO: 134 - protein, 58 aa, Synthetic construct
VDNTLFQAYLQAWYEIFNLPNLNDNQWPAFIESLHEDPSQWANLLAEARRLNDAQAPK

SEQ ID NO: 135 - protein, 58 aa, Synthetic construct
LDNELFQAYLQAWYEIFNLPNLNDNQWPAFIESLHEDPSQWANLLAEARRLNDAQAPK

SEQ ID NO: 136 - protein, 58 aa, Synthetic construct
LDQTLFNAYLQAWYEIFNLPNLNDNQWPAFIESLHEDPSQWANLLAEARRLNDAQAPK

SEQ ID NO: 137 - protein, 58 aa, Synthetic construct
LDQEEFQAYLQAWYEIFNLPNLNDNQWPAFIESLHEDPSQWANLLAEARRLNDAQAPK

SEQ ID NO: 138 - protein, 58 aa, Synthetic construct
LDEEEFQAYLQAWYEIFNLPNLNDNQWPAFIESLHEDPSQWANLLAEARRLNDAQAPK

SEQ ID NO: 139 - protein, 58 aa, Synthetic construct
LDQELFNAYLQAWYEIFNLPNLNDNQWPAFIESLHEDPSQWANLLAEARRLNDAQAPK

SEQ ID NO: 140 - protein, 58 aa, Synthetic construct
VDQTLFQAYLQAWYEIFNLPNLNDNQWPAFIESLHEDPSQWANLLAEARRLNDAQAPK

SEQ ID NO: 141 - protein, 58 aa, Synthetic construct
LDQELFQAYLQAWYEIFNLPNLNDNQWPAFIESLHEDPSQWANLLAEARRLNDAQAPK

SEQ ID NO: 142 - protein, 58 aa, Synthetic construct
LDEELFQAYLQAWYEIFNLPNLNDNQWPAFIESLHEDPSQWANLLAEARRLNDAQAPK

SEQ ID NO: 143 - protein, 58 aa, Synthetic construct
LDQTLFQAYLQAWYEIFNLPNLNDNQWPAFIESLHEDPSQWANLLAEARRLNDAQAPK

SEQ ID NO: 144 - protein, 58 aa, Synthetic construct
LDDTLFQAYLQAWYEIFNLPNLNDNQWPAFIESLHEDPSQWANLLAEARRLNDAQAPK

SEQ ID NO: 145 - protein, 58 aa, Synthetic construct
VDNDEFNAYLQAWYEIFNLPNLNDNQWPAFIESLHEDPSQWANLLAEARRLNDAQAPK

SEQ ID NO: 146 - protein, 58 aa, Synthetic construct
VDNREFIAYLQAWYEIFNLPNLNDNQWPAFIESLHEDPSQWANLLAEARRLNDAQAPK

SEQ ID NO: 147 - protein, 58 aa, Synthetic construct
VDNREFLAYLQAWYEIFNLPNLNDNQWPAFIESLHEDPSQWANLLAEARRLNDAQAPK

SEQ ID NO: 148 - protein, 58 aa, Synthetic construct
VDNREFNLYLQAWYEIFNLPNLNDNQWPAFIESLHEDPSQWANLLAEARRLNDAQAPK

SEQ ID NO: 149 - protein, 58 aa, Synthetic construct
LDNEEFQAYLQAWYEIFNLPNLNDNQWPAFIESLHEDPSQWDNLLEEARRLNDAQAPK

SEQ ID NO: 150 - protein, 58 aa, Synthetic construct
VDQEEFQAYLQAWYEIFNLPNLNDNQWPAFIESLHEDPSQWDNLLAEARRLNDAQAPK

SEQ ID NO: 151 - protein, 58 aa, Synthetic construct
VDNREFVAYLQAWYEIFNLPNLNDNQWPAFIESLHEDPSQWANLLAEARRLNDAQAPK

SEQ ID NO: 152 - protein, 58 aa, Synthetic construct
VDNREFNAYLAAWYEIFNLPNLNDNQWPAFIESLHEDPSQWANLLAEARRLNDAQAPK

SEQ ID NO: 153 - protein, 58 aa, Synthetic construct
VDNTEFQAYLQAWYEIFNLPNLNDNQWPAFIESLHEDPSQWANLLAEARRLNDAQAPK

SEQ ID NO: 154 - protein, 58 aa, Synthetic construct
GEGTEFQAYLQAWYEIFNLPNLNDNQWPAFIESLHEDPSQWANLLAEARRLNDAQAPK

SEQ ID NO: 155 - protein, 58 aa, Synthetic construct
LDEEEFQAYLQAWYEIFNLPNLNDNQWPAFIESLHEDPSQWANLLEEARRLNDAQAPK

SEQ ID NO: 156 - protein, 58 aa, Synthetic construct
LENEEFQAYLQAWYEIFNLPNLNDNQWPAFIESLHEDPSQWANLLAEARRLNDAQAPK

SEQ ID NO: 157 - protein, 58 aa, Synthetic construct
LENEEFQAYLQAWYEIFNLPNLNDNQWPAFIESLHEDPSQWANLLEEARRLNDAQAPK

SEQ ID NO: 158 - protein, 58 aa, Synthetic construct
LENEEFQLYLQAWYEIFNLPNLNDNQWPAFIESLHEDPSQWANLLEEARRLNDAQAPK

SEQ ID NO: 159 - protein, 58 aa, Synthetic construct
LDEEEFQAYLQAWYEIFNLPNLNDNQWPAFIESLHEDPSQWANLLEEARQLNDAQAPK

SEQ ID NO: 160 - protein, 58 aa, Synthetic construct
LDEEEFQAYLQAWYEIFNLPNLNDNQWPAFIESLHEDPSQWTNLLEEARQLNDAQAPK

SEQ ID NO: 161 - protein, 58 aa, Synthetic construct
LDEEEFQAYLQAWYEIFNLPNLNDNQWPAFIESLHEDPSQWTSLLEEARQLNDAQAPK

SEQ ID NO: 162 - protein, 58 aa, Synthetic construct
LDEEEFQAYLQAWYEIFNLPHLNDNQWPAFIESLHEDPSQWANLLEEARRLNDAQAPK

SEQ ID NO: 163 - protein, 58 aa, Synthetic construct
LDEEEFQAYLQAWYEIFNLPNLNDNQWPAFIEALHEDPSQWANLLEEARRLNDAQAPK

SEQ ID NO: 164 - protein, 58 aa, Synthetic construct
LDEEEFQAYLQAWYEIFNLPNLNDNQWPAFIESLHEDPSQAANLLEEARRLNDAQAPK

SEQ ID NO: 165 - protein, 58 aa, Synthetic construct
LDEEEFQAYLQAWYEIFNLPNLNDNQWPAFIEQLHEDPSQWANLLEEARRLNDAQAPK

SEQ ID NO: 166 - protein, 58 aa, Synthetic construct
LDEEEFQAYLQAWYEIFQLPNLNDNQWPAFIESLHEDPSQWANLLAEARRLNDAQAPK

SEQ ID NO: 167 - protein, 58 aa, Synthetic construct
LDEEEFQAYLQAWYEIFNLPNLQDNQWPAFIESLHEDPSQWANLLAEARRLNDAQAPK

SEQ ID NO: 168 - protein, 58 aa, Synthetic construct
LDEEEFQAYLQAWYEIFNLPNLNDQQWPAFIESLHEDPSQWANLLAEARRLNDAQAPK

SEQ ID NO: 169 - protein, 58 aa, Synthetic construct
LDEEEFQAYLQAWYEIFNLPNLNDNQWPAFIESLHEDPSQWAQLLAEARRLNDAQAPK

SEQ ID NO: 170 - protein, 58 aa, Synthetic construct
LDEEEFQAYLQAWYEIFNLPNLNDDQWPAFIESLHEDPSQWANLLEEARRLNDAQAPK

SEQ ID NO: 171 - protein, 58 aa, Synthetic construct
LDEEEFQAYLQAWYEIFNLPNLNDEQWPAFIESLHEDPSQWANLLEEARRLNDAQAPK

SEQ ID NO: 172 - protein, 58 aa, Synthetic construct
LDEEEFQAYLQAWYEIFNLPNLNDQQWPAFIESLHEDPSQWANLLEEARRLNDAQAPK

SEQ ID NO: 173 - protein, 58 aa, Synthetic construct
LDEEEFQAYLQAWYEIFNLPNLNDSQWPAFIESLHEDPSQWANLLEEARRLNDAQAPK

SEQ ID NO: 174 - protein, 58 aa, Synthetic construct
LDEEEFQAYLQAWYEIFNLPNLNDTQWPAFIESLHEDPSQWANLLEEARRLNDAQAPK

SEQ ID NO: 175 - protein, 58 aa, Synthetic construct
LDEEEFQAYLQAWYEIFNLPNLNDHQWPAFIESLHEDPSQWANLLEEARRLNDAQAPK

SEQ ID NO: 176 - protein, 58 aa, Synthetic construct
LDEEEFQAYLQAWYEIFNLPNLNDAQWPAFIESLHEDPSQWANLLEEARRLNDAQAPK

SEQ ID NO: 177 - protein, 58 aa, Synthetic construct
LDEEEFQAYLQAWYEIFNLPNLNDLQWPAFIESLHEDPSQWANLLEEARRLNDAQAPK

SEQ ID NO: 178 - protein, 58 aa, Synthetic construct
LDEEEFQAYLQAWYEIFNLPNLNDNQWPAFIESLHEDPSQWAELLEEARRLNDAQAPK

SEQ ID NO: 179 - protein, 58 aa, Synthetic construct
LDEEEFQAYLQAWYEIFNLPNLNDNQWPAFIESLHEDPSQWAQLLEEARRLNDAQAPK

SEQ ID NO: 180 - protein, 58 aa, Synthetic construct
LDEEEFQAYLQAWYEIFNLPNLNDEQWPAFIESLHEDPSQWAELLEEARRLNDAQAPK

SEQ ID NO: 181 - protein, 58 aa, Synthetic construct
LDEEEFQAYLQAWYEIFNLPNLNDEQWPAFIESLHEDPSQWAQLLEEARRLNDAQAPK

SEQ ID NO: 182 - protein, 58 aa, Synthetic construct
LDEEEFQAYLQAWYEIFNLPNLNDQQWPAFIESLHEDPSQWAELLEEARRLNDAQAPK

SEQ ID NO: 183 - protein, 58 aa, Synthetic construct
LDEEEFQAYLQAWYEIFNLPNLNDQQWPAFIESLHEDPSQWAQLLEEARRLNDAQAPK

SEQ ID NO: 184 - protein, 58 aa, Synthetic construct
LDEEEFQAYLQAWYEIFNLPYLNDNQWPAFIESLHEDPSQWANLLEEARRLNDAQAPK

SEQ ID NO: 185 - protein, 58 aa, Synthetic construct
LDEEEFQAYLQAWYEIFNLPNLTDNQWPAFIESLHEDPSQWAELLEEARRLNDAQAPK

SEQ ID NO: 186 - protein, 58 aa, Synthetic construct
LDEEEFQAYLQAWYEIFNLPNLTDNQWPAFIESLHEDPSQWAQLLEEARRLNDAQAPK

SEQ ID NO: 187 - protein, 58 aa, Synthetic construct
LDEEEFQAYLQAWYEIFNLPNLTDEQWPAFIESLHEDPSQWANLLEEARRLNDAQAPK

SEQ ID NO: 188 - protein, 58 aa, Synthetic construct
LDEEEFQAYLQAWYEIFNLPNLTDEQWPAFIESLHEDPSQWAELLEEARRLNDAQAPK

SEQ ID NO: 189 - protein, 58 aa, Synthetic construct
LDEEEFQAYLQAWYEIFNLPNLTDEQWPAFIESLHEDPSQWAQLLEEARRLNDAQAPK

SEQ ID NO: 190 - protein, 58 aa, Synthetic construct
LDEEEFQAYLQAWYEIFNLPNLTDQQWPAFIESLHEDPSQWANLLEEARRLNDAQAPK

SEQ ID NO: 191 - protein, 58 aa, Synthetic construct
LDEEEFQAYLQAWYEIFNLPNLTDQQWPAFIESLHEDPSQWAELLEEARRLNDAQAPK

SEQ ID NO: 192 - protein, 58 aa, Synthetic construct
LDEEEFQAYLQAWYEIFNLPNLTDQQWPAFIESLHEDPSQWAQLLEEARRLNDAQAPK

SEQ ID NO: 193 - protein, 58 aa, Synthetic construct
LDEEEFQAYLQAWYEIFNLPNLNDNQWPAFIESLHEEPSQWANLLEEARRLNDAQAPK

SEQ ID NO: 194 - protein, 58 aa, Synthetic construct
LDEEEFQAYLQAWYEIFNLPNLNDNQWPAFIESLHEEPSQWAELLEEARRLNDAQAPK

SEQ ID NO: 195 - protein, 58 aa, Synthetic construct
LDEEEFQAYLQAWYEIFNLPNLNDNQWPAFIESLHEEPSQWAQLLEEARRLNDAQAPK

SEQ ID NO: 196 - protein, 58 aa, Synthetic construct
LDEEEFQAYLQAWYEIFNLPNLNDEQWPAFIESLHEEPSQWANLLEEARRLNDAQAPK

SEQ ID NO: 197 - protein, 58 aa, Synthetic construct
LDEEEFQAYLQAWYEIFNLPNLNDEQWPAFIESLHEEPSQWAELLEEARRLNDAQAPK

SEQ ID NO: 198 - protein, 58 aa, Synthetic construct
LDEEEFQAYLQAWYEIFNLPNLNDEQWPAFIESLHEEPSQWAQLLEEARRLNDAQAPK

SEQ ID NO: 199 - protein, 58 aa, Synthetic construct
LDEEEFQAYLQAWYEIFNLPNLNDQQWPAFIESLHEEPSQWANLLEEARRLNDAQAPK

SEQ ID NO: 200 - protein, 58 aa, Synthetic construct
LDEEEFQAYLQAWYEIFNLPNLNDQQWPAFIESLHEEPSQWAELLEEARRLNDAQAPK

SEQ ID NO: 201 - protein, 58 aa, Synthetic construct
LDEEEFQAYLQAWYEIFNLPNLNDDQWPAFIESLHEDPSQWAQLLEEARRLNDAQAPK

SEQ ID NO: 202 - protein, 58 aa, Synthetic construct
LDEEEFQAYLQAWYEIFNLPNLSDNQWPAFIESLHEDPSQWANLLEEARRLNDAQAPK

SEQ ID NO: 203 - protein, 58 aa, Synthetic construct
LDEEEFQAYLQAWYEIFNLPNLNDSQWPAFIESLHEDPSQWAQLLEEARRLNDAQAPK

SEQ ID NO: 204 - protein, 58 aa, Synthetic construct
LDEEEFQAYLQAWYEIFNLPNLNDTQWPAFIESLHEDPSQWAQLLEEARRLNDAQAPK

SEQ ID NO: 205 - protein, 58 aa, Synthetic construct
LDEEEFQAYLQAWYEIFNLPNLNDAQWPAFIESLHEDPSQWAQLLEEARRLNDAQAPK

SEQ ID NO: 206 - protein, 58 aa, Synthetic construct
LDEEEFQAYLQAWYEIFNLPNLNDLQWPAFIESLHEDPSQWAQLLEEARRLNDAQAPK

SEQ ID NO: 207 - protein, 58 aa, Synthetic construct
LDEEEFQAYLQAWYEIFNLPNLNDGQWPAFIESLHEDPSQWAQLLEEARRLNDAQAPK

SEQ ID NO: 208 - protein, 58 aa, Synthetic construct
LDEEEFQAYLQAWYEIFNLPNLNDDQWPAFIESLHEDPSQWAELLEEARRLNDAQAPK

SEQ ID NO: 209 - protein, 58 aa, Synthetic construct
LDEEEFQAYLQAWYEIFQLPNLNDNQWPAFIESLHEDPSQWANLLEEARRLNDAQAPK

SEQ ID NO: 210 - protein, 58 aa, Synthetic construct
LDEEEFQAYLQAWYEIFNLPNLNDNQWPAFIESLHEDPSQWASLLEEARRLNDAQAPK

SEQ ID NO: 211 - protein, 58 aa, Synthetic construct
LDEEEFQAYLQAWYEIFNLPNLNDSQWPAFIESLHEDPSQWAELLEEARRLNDAQAPK

SEQ ID NO: 212 - protein, 58 aa, Synthetic construct
LDEEEFQAYLQAWYEIFNLPNLNDTQWPAFIESLHEDPSQWAELLEEARRLNDAQAPK

SEQ ID NO: 213 - protein, 58 aa, Synthetic construct
LDEEEFQAYLQAWYEIFNLPNLNDAQWPAFIESLHEDPSQWAELLEEARRLNDAQAPK

SEQ ID NO: 214 - protein, 58 aa, Synthetic construct
LDEEEFQAYLQAWYEIFNLPNLNDLQWPAFIESLHEDPSQWAELLEEARRLNDAQAPK

SEQ ID NO: 215 - protein, 58 aa, Synthetic construct
LDEEEFQAYLQAWYEIFNLPNLNDGQWPAFIESLHEDPSQWAELLEEARRLNDAQAPK

SEQ ID NO: 216 - protein, 58 aa, Synthetic construct
LDEEEFAAYLQAWYEIFNLPNLNDNQWPAFIESLHEDPSQWANLLEEARRLNDAQAPK

SEQ ID NO: 217 - protein, 58 aa, Synthetic construct
LDEEEFQAYVQAWYEIFNLPNLNDNQWPAFIESLHEDPSQWANLLEEARRLNDAQAPK

SEQ ID NO: 218 - protein, 58 aa, Synthetic construct
LDEEEFQAYIQAWYEIFNLPNLNDNQWPAFIESLHEDPSQWANLLEEARRLNDAQAPK

SEQ ID NO: 219 - protein, 58 aa, Synthetic construct
LDEEEFQAYFQAWYEIFNLPNLNDNQWPAFIESLHEDPSQWANLLEEARRLNDAQAPK

SEQ ID NO: 220 - protein, 58 aa, Synthetic construct
LDEEEFQAYLQAWFEIFNLPNLNDNQWPAFIESLHEDPSQWANLLEEARRLNDAQAPK

SEQ ID NO: 221 - protein, 58 aa, Synthetic construct
LDEEEFQAYLQAWWEIFNLPNLNDNQWPAFIESLHEDPSQWANLLEEARRLNDAQAPK

SEQ ID NO: 222 - protein, 58 aa, Synthetic construct
LDEEEFQAYLQAWHEIFNLPNLNDNQWPAFIESLHEDPSQWANLLEEARRLNDAQAPK

SEQ ID NO: 223 - protein, 58 aa, Synthetic construct
LDEEEFQAYLQAWYEIFNLPQLNDNQWPAFIESLHEDPSQWANLLEEARRLNDAQAPK

SEQ ID NO: 224 - protein, 58 aa, Synthetic construct
LDEEEFQAYLQAWYEIFNLPNLQDNQWPAFIESLHEDPSQWANLLEEARRLNDAQAPK

SEQ ID NO: 225 - protein, 58 aa, Synthetic construct
VDEEEFQAYLQAWYEIFNLPNLNDNQWPAFIESLHEDPSQWANLLEEARRLNDAQAPK

SEQ ID NO: 226 - protein, 58 aa, Synthetic construct
VDEEEFQAYLQAWYEIFNLPNLNDQQWPAFIESLHEDPSQWANLLEEARRLNDAQAPK

SEQ ID NO: 227 - protein, 58 aa, Synthetic construct
LEEEEFQAYLQAWYEIFNLPNLNDNQWPAFIESLHEDPSQWANLLEEARRLNDAQAPK

SEQ ID NO: 228 - protein, 58 aa, Synthetic construct
LEEEEFQAYLQAWYEIFNLPNLNDNQWPAFIESLHEEPSQWANLLEEARRLNDAQAPK

SEQ ID NO: 229 - protein, 58 aa, Synthetic construct
LEEEEFQAYLQAWYEIFNLPNLNDQQWPAFIESLHEEPSQWANLLEEARRLNDAQAPK

SEQ ID NO: 230 - protein, 58 aa, Synthetic construct
LDEEEFQAYLQAWYEIFNLPNLNDEQWPAFIESLHEDPSQWANLLAEARRLNDAQAPK

SEQ ID NO: 231 - protein, 58 aa, Synthetic construct
LDEEEFQAYLQAWYEIFNLPNLNDAQWPAFIESLHEDPSQWANLLAEARRLNDAQAPK

SEQ ID NO: 232 - protein, 58 aa, Synthetic construct
LDEEEFQAYLQAWYEIFNLPNLQDQQWPAFIESLHEDPSQWANLLEEARRLNDAQAPK

SEQ ID NO: 233 - protein, 58 aa, Synthetic construct
LDEEEFQAYLQAWYEIFNLPNLNDQQWPAFIESLHEDPSQWAQLLAEARRLNDAQAPK

SEQ ID NO: 234 - protein, 58 aa, Synthetic construct
LDEEEFQAYLQAWYEIFNLPNLNDQQWPAFIESLHEDPSQWANLLAEARQLNDAQAPK

SEQ ID NO: 235 - protein, 58 aa, Synthetic construct
LDEEEFQAYLQAWYEIFNLPHLNDQQWPAFIESLHEDPSQWANLLEEARRLNDAQAPK

SEQ ID NO: 236 - protein, 58 aa, Synthetic construct
LDEEEFQAYLQAWYEIFNLPNLNDQQWPAFIESLHEDPSQWANLLAEARRLNEAQAPK

SEQ ID NO: 237 - protein, 58 aa, Synthetic construct
LDEEEFQAYLQAWYEIFNLPNLNDQQWPAFIESLHEDPSQWANLLEEARRLNEAQAPK

SEQ ID NO: 238 - protein, 58 aa, Synthetic construct
LDEEEFQAYLQAWYEIFNLPNLNDQQWPAFIESLHEDPSQWANLLAEARQLNEAQAPK

SEQ ID NO: 239 - protein, 58 aa, Synthetic construct
LDEEEFQAYLQAWYEIFNLPNLDDAQWPAFIESLHEDPSQWANLLEEARRLNDAQAPK

SEQ ID NO: 240 - protein, 58 aa, Synthetic construct
LDEEEFQAYLQAWYEIFNLPNLNDQQWPAFIESLHEDPSQWANLLEEARQLNDAQAPK

SEQ ID NO: 241 - protein, 58 aa, Synthetic construct
LDEEEFQAYLQAWYEIFNLPNLTDAQWPAFIESLHEDPSQWANLLEEARRLNDAQAPK

SEQ ID NO: 242 - protein, 58 aa, Synthetic construct
LDEEEFQAYLQAWYEIFNLPNLTDAQWPAFIESLHEDPSQWAQLLEEARRLNDAQAPK

SEQ ID NO: 243 - protein, 58 aa, Synthetic construct
LDEEEFQAYLQAWYEIFNLPNLDDQQWPAFIESLHEDPSQWANLLAEARRLNDAQAPK

SEQ ID NO: 244 - protein, 58 aa, Synthetic construct
LDEEEFQAYLQAWYEIFNLPNLDDQQWPAFIESLHEDPSQWANLLEEARRLNDAQAPK

SEQ ID NO: 245 - protein, 58 aa, Synthetic construct
LEEEEFQAYLQAWYEIFNLPNLNDAQWPAFIESLHEDPSQWANLLEEARRLNDAQAPK

SEQ ID NO: 246 - protein, 58 aa, Synthetic construct
LQEEEFQAYLQAWYEIFNLPNLNDAQWPAFIESLHEDPSQWANLLEEARRLNDAQAPK

SEQ ID NO: 247 - protein, 58 aa, Synthetic construct
LEEEEFQAYLQAWYEIFNLPHLNDAQWPAFIESLHEDPSQWANLLEEARRLNDAQAPK

SEQ ID NO: 248 - protein, 58 aa, Synthetic construct
LEEEEFQAYLQAWYEIFNLPNLTDAQWPAFIESLHEDPSQWANLLEEARRLNDAQAPK

SEQ ID NO: 249 - protein, 58 aa, Synthetic construct
LEEEEFQAYLQAWYEIFNLPNLNDQQWPAFIESLHEDPSQWANLLEEARRLNDAQAPK

SEQ ID NO: 250 - protein, 58 aa, Synthetic construct
LEEEEFQAYLQAWYEIFNLPNLNDYQWPAFIESLHEDPSQWANLLEEARRLNDAQAPK

SEQ ID NO: 251 - protein, 58 aa, Synthetic construct
LEEEEFQAYLQAWYEIFNLPNLNDLQWPAFIESLHEDPSQWANLLEEARRLNDAQAPK

SEQ ID NO: 252 - protein, 58 aa, Synthetic construct
LEEEEFQAYLQAWYEIFNLPNLNDAQWPAFIESLHEDPSQWASLLEEARRLNDAQAPK

SEQ ID NO: 253 - protein, 58 aa, Synthetic construct
LEEEEFQAYLQAWYEIFNLPNLNDAQWPAFIESLHEDPSQWAELLEEARRLNDAQAPK

SEQ ID NO: 254 - protein, 58 aa, Synthetic construct
LEEEEFQAYLQAWYEIFNLPNLNDAQWPAFIESLHEDPSQWANLLEEARRLNEAQAPK

SEQ ID NO: 255 - protein, 58 aa, Synthetic construct
LEEEEFQAYLQAWYEIFNLPNLNDAQWPAFIESLHEDPSQWANLLEEARRLNTAQAPK

SEQ ID NO: 256 - protein, 58 aa, Synthetic construct
LEEEEFQAYLQAWYEIFNLPNLNDAQWPAFIESLHEDPSQWANLLEEARRLNDIQAPK

SEQ ID NO: 257 - protein, 58 aa, Synthetic construct
LEEEEFQAYLQAWYEIFNLPHLTDQQWPAFIESLHEEPSQWAELLEEARRLNEAQAPK

SEQ ID NO: 258 - protein, 58 aa, Synthetic construct
LEEEEFQAYLQAWYEIFNLPHLTDQQWPAFIESLHEEPSQWAQLLEEARRLNEAQAPK

SEQ ID NO: 259 - protein, 58 aa, Synthetic construct
LEEEEFQAYLQAWYEIFNLPNLTDQQWPAFIESLHEDPSQWAELLEEARRLNDAQAPK

SEQ ID NO: 260 - protein, 58 aa, Synthetic construct
LEEEEFQAYLQAWYEIFNLPNLTDAQWPAFIESLHEDPSQWAELLEEARRLNDAQAPK

SEQ ID NO: 261 - protein, 58 aa, Synthetic construct
LEEEEFQAYLQAWYEIFNLPNLTDAQWPAFIESLHEEPSQWAELLEEARRLNDAQAPK

SEQ ID NO: 262 - protein, 58 aa, Synthetic construct
LEEEEFQAYLQAWYEIFNLPNLTDYQWPAFIESLHEEPSQWANLLEEARRLNDAQAPK

SEQ ID NO: 263 - protein, 58 aa, Synthetic construct
LEEEEFQAYLQAWYEIFNLPNLTDQQWPAFIESLHEDPSQWAQLLEEARRLNEAQAPK

SEQ ID NO: 264 - protein, 58 aa, Synthetic construct
LEEEEFQAYLQAWYEIFNLPNLNDQQWPAFIESLHEDPSQWAQLLEEARRLNEAQAPK

SEQ ID NO: 265 - protein, 58 aa, Synthetic construct
LEEEEFQAYLQAWYEIFNLPNLTDAQWPAFIESLHEDPSQWAQLLEEARRLNEAQAPK

SEQ ID NO: 266 - protein, 58 aa, Synthetic construct
LEEEEFQAYLQAWYEIFNLPNLTDYQWPAFIESLHEDPSQWANLLEEARRLNEAQAPK

SEQ ID NO: 267 - protein, 58 aa, Synthetic construct
LEEEEFQAYLQAWYEIFNLPHLNDQQWPAFIESLHEEPSQWAQLLEEARRLNEAQAPK

SEQ ID NO: 268 - protein, 58 aa, Synthetic construct
LEEEEFQAYLQAWYEIFNLPHLNDQQWPAFIESLHEEPSQWAQLLEEARRLNDAQAPK

SEQ ID NO: 269 - protein, 58 aa, Synthetic construct
LEEEEFQAYLQAWYEIFNLPHLNDQQWPAFIESLHEEPSQWANLLEEARRLNEAQAPK

SEQ ID NO: 270 - protein, 58 aa, Synthetic construct
LEEEEFQAYLQAWYEIFNLPHLNDQQWPAFIESLHEDPSQWAQLLEEARRLNEAQAPK

SEQ ID NO: 271 - protein, 58 aa, Synthetic construct
LEEEEFQAYLQAWYEIFNLPHLNDQQWPAFIESLHEDPSQWAQLLEEARRLNDAQAPK

SEQ ID NO: 272 - protein, 58 aa, Synthetic construct
LEEEEFQAYLQAWYEIFTLPHLTDQQWPAFIESLHEEPSQWAELLEEARRLNEAQAPK

SEQ ID NO: 273 - protein, 58 aa, Synthetic construct
LEEEEFQAYLQAWYEIFTLPHLTDQQWPAFIESLHEEPSQWAQLLEEARRLNEAQAPK

SEQ ID NO: 274 - protein, 58 aa, Synthetic construct
VDQEEFQAYLQAWYEIFNLPCLNDNQWPAFIESLHEDPSQWDNLLEEARRLNDAQCPK

SEQ ID NO: 275 - protein, 58 aa, Synthetic construct
LDNEEFQAYLQAWYEIFNLPCLNDNQWPAFIESLHEDPSQWDNLLEEARRLNDAQCPK

SEQ ID NO: 276 - protein, 58 aa, Synthetic construct
LDNDEFILYLQAWYEIFNLPCLNDNQWPAFIESLHDDPSQWANLLAEARRLNDAQCPK

SEQ ID NO: 277 - protein, 58 aa, Synthetic construct
LDNDEFILYLQAWYEIFNLPCLNDNQWPAFIESLHDDPSQWANLLEEARRLNDAQCPK

SEQ ID NO: 278 - protein, 58 aa, Synthetic construct
LDNDEFQLYLQAWYEIFNLPCLNDNQWPAFIESLHDDPSQWANLLEEARRLNDAQCPK

SEQ ID NO: 279 - protein, 58 aa, Synthetic construct
LDEEEFQAYLQAWYEIFNLPCLNDQQWPAFIESLHEDPSQWANLLEEARRLNDAQCPK

SEQ ID NO: 280 - protein, 58 aa, Synthetic construct
LDEEEFQAYLQAWYEIFNLPCLTDSQWPAFIESLHEDPSQWANLLEEARRLNDAQCPK

(19) United States
(12) Patent Application Publication
(10) Pub. No.: US 2026/0274901 A1
(43) Pub. Date: Sep. 17, 2026

(54) ANTAGONISTS OF HUMAN FGF23

(71) Applicant: Novo Nordisk A/S, Bagsvaerd (DK)

(72) Inventors: Christian Wenzel Tornoee, Lyngby (DK); Camille Villequey, Ballerup (DK); Marina Kasimova, Broenshoei (DK); Franziska Schoeppe, Koebenhavn S (DK)

(21) Appl. No.: 19/355,693

(22) Filed: Oct. 10, 2025

(30) Foreign Application Priority Data
Oct. 11, 2024 (EP) 24206126.5

(51) Int. Cl. C07K 14/31; A61K 38/00

(52) U.S. Cl. CPC C07K 14/31; A61K 38/00

(57) ABSTRACT

The present invention relates to novel hFGF23 antagonistic polypeptides and long-acting derivatives thereof, and their use in medicine and in particular for inhibiting hFGF23-induced signalling in patients in need thereof, such as patients suffering from hypophosphatemic disorders, such as X-linked hypophosphatemic rickets (XLH), as well as pharmaceutical compositions comprising such compounds.

### TECHNICAL FIELD

Human fibroblast growth factor 23 (hFGF23) antagonists and compositions comprising such compounds for use in medicine.

### CROSS-REFERENCE TO RELATED APPLICATIONS

This application claims priority under 35 U.S.C. § 119 to European Patent Application 24206126.5, filed Oct. 11, 2024; the contents of which are incorporated herein by reference.

### INCORPORATION-BY-REFERENCE OF THE SEQUENCE LISTING

The instant application contains a Sequence Listing which has been submitted in XML format via USPTO patent electronic filing system and is hereby incorporated by reference in its entirety. Said XML file, created on Mar. 19, 2026, is named “240007US01_sequence_listing_rev.xml”, and is 273,067 bytes in size.

### BACKGROUND

Human fibroblast growth factor 23 (hFGF23) is a human protein with 251 amino acid residues which acts by binding to its receptor complex, primarily fibroblast growth factor receptor 1 isoform c (FGFR1c) and alpha-klotho. Activation of FGFR1c-alpha-klotho by hFGF23 in the kidney leads to the inhibition of sodium-phosphate cotransporters in the proximal tubules, resulting in decreased reabsorption of phosphate, thereby increasing phosphate excretion in urine and reducing phosphate concentration in blood. Therefore, inhibition of hFGF23 activity can be used in the treatment of certain hypophosphatemic disorders. X-linked hypophosphatemic rickets (henceforth referred to as XLH) is one such disease where the hFGF23 concentration in the blood is elevated due to loss of function mutations in the PHEX (phosphate-regulating endopeptidase homologue, X-linked) gene. Until recently, the sole therapy for chronic hypophosphatemia in XLH has been supplementation with high doses of oral phosphate and active vitamin D (referred to as conventional therapy). However, conventional therapy is burdensome, poorly tolerated, has limited efficacy and is associated with dangerous adverse events. In 2018, a monoclonal IgG1 anti-hFGF23 antibody, burosumab, was approved for the treatment of XLH. Inhibition of hFGF23 by burosumab increases 1,25 dihydroxyvitamin D levels and the renal reabsorption of phosphate, resulting in improved blood phosphate levels in XLH patients (WO2008/099969).

Despite burosumab's ability to improve phosphate levels in blood, many patients still experience hypophosphatemia during treatment, and burosumab has a heavy treatment burden. In paediatric patients treated with burosumab, many do not achieve a phosphate concentration within the age-appropriate reference range. For adults on burosumab, in addition to a subset of patients not reaching the normal range, phosphate levels are not stable over the course of the treatment and so many patients experience hypophosphatemia during the latter time between doses. High dose and large injection volumes needed for burosumab treatment makes it poorly compatible with self-administration devices, such as injectable pens. In most cases, burosumab is administered by a healthcare professional, which imposes a heavy burden on the patient and caregivers. In addition, the injection itself is burdensome and can be painful due to high volume and the process of loading the drug into a syringe from a vial. Each dose requires up to 3 ml for adults, and typically 1-2 ml for children. Hence, there is a need in the art for improved hFGF23 antagonistic compounds.

### SUMMARY

The present invention relates to compounds in the form of hFGF23 antagonistic polypeptides and derivatives thereof comprising a protraction moiety (i.e. a moiety extending the half-life in blood) which are capable of binding to hFGF23 and antagonizing hFGF23-induced signalling activity. The hFGF23 antagonistic polypeptides and derivatives thereof as disclosed herein are designed to provide an efficacious treatment of hypophosphatemic disorders, such as X-linked hypophosphatemic rickets (XLH).

In one aspect the invention relates to hFGF23 antagonistic polypeptides, which are capable of binding an epitope comprising the amino acid residues selected from the group consisting of:
a) R76, T86, R91 and R114;
b) M74, R76, T86, R91, F108, P110, R114, L166, I167 and N170;
c) R76, T86, R91, Y93, Y107, R114, F169 and N170;
d) W36, R76, T86, R91, Y107, R114, 1167, F169 and N170; and
e) R76, T86, R91, R114, L166 and F169;
on hFGF23 according to SEQ ID NO:1 as determined at 3.5 Å.

In another aspect the invention relates to hFGF23 antagonistic polypeptides, which are capable of binding hFGF23 according to SEQ ID NO:1, comprising a binding motif (BM) which consists of an amino acid residue sequence selected from

i)
XₐX_{b}X_{c}AWX_{d}EIXₑX_{f}X_{g}PXₕLXᵢDXⱼQWPAFIEXₖLH
according to SEQ ID NO: 2

wherein, independently of each other,
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S; and
ii) an amino acid residue sequence having at least 93% identity to the sequence defined in i).

In another aspect the invention relates to hFGF23 antagonistic polypeptides, which comprise a binding motif (BM) as set out above, wherein said polypeptides comprises an amino acid residue sequence selected from

a)
X₁X₂X₃X₄X₅X₆X₇X₈-(BM)-X₃₆X₃₇PSQX₄₁X₄₂X₄₃LLX₄₆EARX₅₀LX₅₂X₅₃
X₅₄QX₅₆X₅₇X₅₈ according to SEQ ID NO: 3,

wherein (BM) consists of an amino acid residue sequence as defined above,
wherein, independently of each other,
X₁ is G, I, L, N or V;
X₂ is D, E or Q;
X₃ is D, E, G, N or Q;
X₄ is D, E, H, N, R or T;
X₅ is E, I, L, T or V;
X₆ is F, W or Y;
X₇ is A, G, H, I, L, N, P, Q, R, T or V;
X₈ is A, L or E;
X₃₆ is A, D or E;
X₃₇ is D or E;
X₄₁ is A, R or W;
X₄₂ is A, D or T;
X₄₃ is E, N, Q or S;
X₄₆ is A, E or K;
X₅₀ is Q or R;
X₅₂ is N or E;
X₅₃ is D, E, K or T;
X₅₄ is A or I;
X₅₆ is A or C;
X₅₇ is K or P;
X₅₈ is C, K or E; and
b) an amino acid residue sequence having at least 93% identity to a sequence defined in a).

In one aspect the invention relates to hFGF23 antagonistic polypeptides such as those represented by Chem. 89, Chem. 106, Chem. 107, Chem. 111, Chem. 197, Chem. 201, Chem. 204, Chem. 206, Chem. 207 and Chem. 208 herein.

In another aspect the invention relates to hFGF23 antagonistic polypeptides as described above wherein a protraction moiety is attached to said polypeptides, such to arrive at hFGF23 antagonistic polypeptide derivatives.

In another aspect the invention relates to a compound selected from the group consisting of:

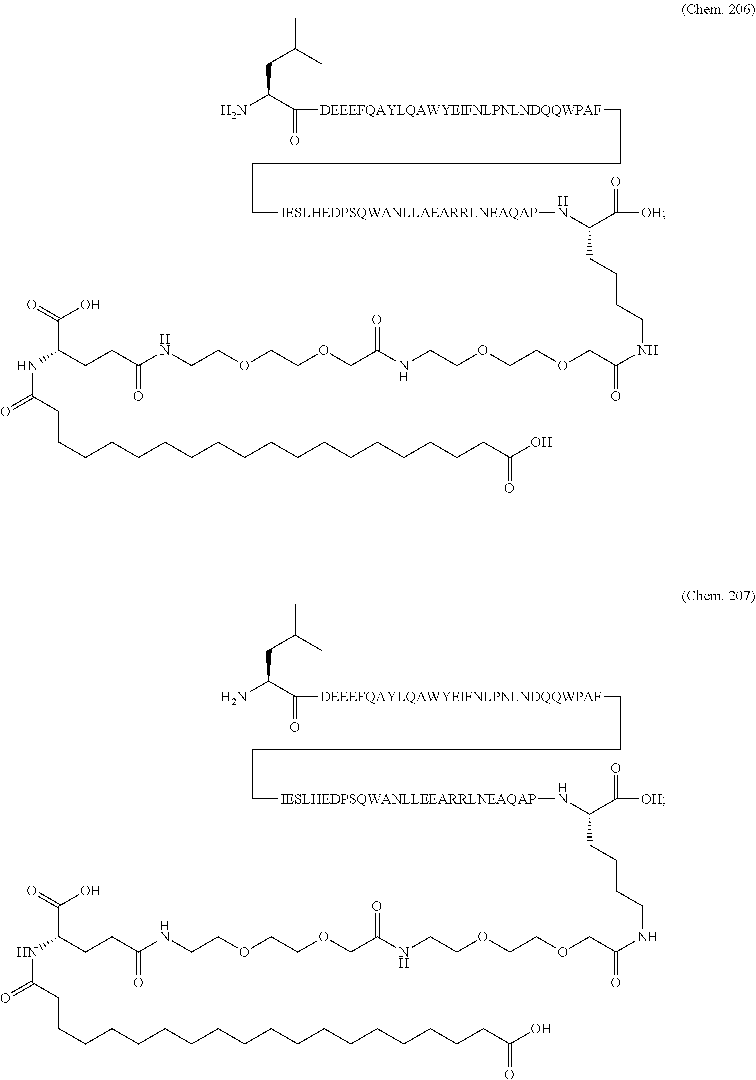

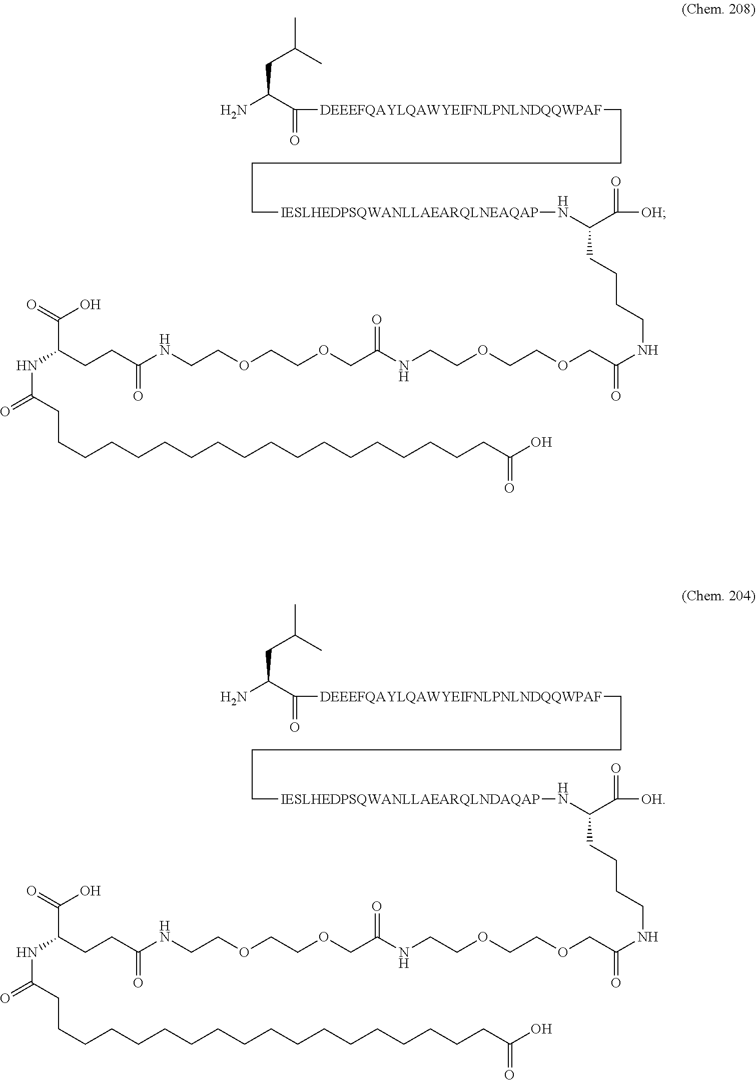

In another aspect the invention relates to further compounds as disclosed herein.

In another aspect the invention relates to polynucleotides, vectors and host cells suitable for producing the hFGF23 antagonistic polypeptides, derivatives thereof, and compounds as described above.

In another aspect the invention relates to pharmaceutical compositions comprising hFGF23 antagonistic polypeptides or derivatives thereof, or compounds as described above.

In another aspect the invention relates to kits comprising hFGF23 antagonistic polypeptides or derivatives thereof, compounds or compositions as described above, and instructions for use.

In another aspect the invention relates to medicinal use of the hFGF23 antagonistic polypeptides or derivatives thereof, compounds, compositions or kits as described above, and in particular for use in the treatment of a hypophosphatemic disease, such as—but not limited to—X-linked hypophosphatemic rickets (XLH).

The invention may also solve further problems that will be apparent from the disclosure of the exemplary embodiments.

### BRIEF DESCRIPTION OF THE FIGURES

FIG. 1 shows plasma phosphate levels after i.v. dosing of compounds to minipigs (Chem. 197, Chem. 201, Chem. 204).

FIG. 2 shows plasma phosphate levels after s.c. dosing of compounds to male Beagle dogs (Chem. 204, 206, 207, 208).

FIG. 3 shows plasma phosphate levels after i.v. dosing of compounds to minipigs (Chem. 206).

### BRIEF DESCRIPTION OF THE SEQUENCES

SEQ ID NO:1 represents the amino acid residue sequence of mature human FGF23 (hFGF23) after removal of the 24-amino acid signal peptide from the full-length human FGF23.

SEQ ID NO:2 represents the amino acid residue sequence of binding motifs of the invention which comprises positions with fixed or non-fixed residues.

SEQ ID NO:3 represents the amino acid residue sequence of hFGF23 antagonistic polypeptides of the invention which comprises positions with fixed or non-fixed residues.

SEQ ID NO:4 represents the amino acid residue sequence of the hFGF23 fragment used in Example 8 herein.

SEQ ID NO:5 represents the amino acid residue sequence of the hFGF23 variant used in Example 2 herein.

SEQ ID NO:6 represents the amino acid residue sequence of the hFGF23 variant used in Example 3 herein.

SEQ ID NO:7 represents the amino acid residue sequence of the heavy chain of burosumab.

SEQ ID NO:8 represents the amino acid residue sequence of the light chain of burosumab.

SEQ ID NO:9 represents the amino acid residue sequence of a peptide protractor.

SEQ ID NO:10 represents the amino acid residue sequence of a hFGF23 antagonistic polypeptide used in Example 8 herein.

SEQ ID NOs:11-52 represent the amino acid residue sequences of hFGF23 antagonistic polypeptide binding motifs (BM).

SEQ ID NOs:53-280 represent the amino acid residue sequences of hFGF23 antagonistic polypeptides.

### DESCRIPTION

The present invention relates to compounds capable of binding to human FGF23 (hFGF23) and capable of antagonizing its biological activity. hFGF23 (as represented by SEQ ID NO:1 herein) is involved in metabolism of phosphate and calcium in the body.

Inhibition of hFGF23 activity can be used in the treatment of certain hypophosphatemic disorders, such as X-linked hypophosphatemic rickets (XLH) where the hFGF23 concentration in the blood is elevated due to loss of function mutations in the PHEX (phosphate-regulating endopeptidase homologue, X-linked) gene. Further diseases that can be prevented or treated by the compounds of the present invention include diseases characterized by excessive activity of hFGF23 such as tumor-induced osteomalacia, autosomal dominant hypophosphatemic rickets (ADHR), fibrous dysplasia and McCune-Albright syndrome.

The hFGF23 antagonistic polypeptides as disclosed herein comprise a three-helix bundle protein domain derived from the Z domain of staphylococcal protein A. By randomizing amino acids on two of the three helices, large libraries were constructed, from which potent binders were identified. The compounds of the invention are thus designed to bind to hFGF23 such to antagonize hFGF23-induced signalling activity with high potency and provide efficacious treatment of hypophosphatemia and to be compatible with low burden administration devices that are amenable to self- or caregiver drug delivery at home. In one embodiment, the hFGF23 antagonistic polypeptides as disclosed herein specifically binds to hFGF23 such to reduce hFGF23-induced signalling mediated by both FGFR1c as receptor and alpha-klotho (KL) as co-receptor thereby, for example, increasing phosphate levels in blood.

The compounds as disclosed herein preferably have a molecular mass between 5 and 10 kDa, such as between 7 and 8 kDa, which is substantially smaller than the antibody designated burosumab (around 147 kDa) and have an in vivo therapeutically useful half-life in blood anticipated to allow for a convenient dosing frequency that provides an efficacious reduction of hFGF23-induced signalling. The presence of a protraction moiety serves to increase the half-life in blood of the compounds disclosed herein.

Without being bound by theory this means that, for a potency close or equivalent to burosumab in nmole/kg, a lower dose in mg/kg and/or lower injection volume can be used, which in turn would be compatible with a self-administration device such as an injectable pen. An improved phosphate upregulation with less peak-to-through variation is, moreover, expected.

Thus, the molecular mass of the compounds expectedly allows for high concentration in a pharmaceutical formulation. The pharmacokinetic properties of the compounds as disclosed herein allow for a favourable drug exposure profile that is expected to minimize phosphate variation between doses, which is expected to reduce hypophosphatemia between doses and to consequently improve disease outcomes. In addition, the compounds as disclosed herein have an efficacy expected to be significantly better than burosumab per mg when used in patients in need thereof, such as patients suffering from XLH. The increased efficacy of the compounds should allow them to provide greater magnitudes of phosphate improvements beyond what burosumab can achieve, thus improving rates of phosphate normalization in said patients.

In one aspect the compounds of the invention are characterized by reference to their target binding site, i.e. the amino acid residues on hFGF23 making up the key epitope residues or full set of epitope residues for the hFGF23 antagonistic polypeptides. Thus, in some embodiments the invention relates to hFGF23 antagonistic polypeptides, which are capable of binding an epitope comprising the amino acid residues selected from the group consisting of:
a) R76, T86, R91 and R114;
b) M74, R76, T86, R91, F108, P110, R114, L166, I167 and N170;
c) R76, T86, R91, Y93, Y107, R114, F169 and N170;
d) W36, R76, T86, R91, Y107, R114, 1167, F169 and N170; and
e) R76, T86, R91, R114, L166 and F169;
on hFGF23 according to SEQ ID NO:1 as determined at 3.5 Å.

In a preferred embodiment a compound of the invention is a hFGF23 antagonistic polypeptide, which is capable of binding an epitope comprising amino acid residues M74, R76, T86, R91, F108, P110, R114, L166, 1167, N170 on hFGF23 according to SEQ ID NO:1 as determined at 3.5 Å.

In one aspect the compounds of the invention are characterized by reference to their binding motifs (BM). In one such embodiment the compounds can be described as hFGF23 antagonistic polypeptides, which are capable of binding hFGF23 according to SEQ ID NO:1, comprising a binding motif (BM) which consists of an amino acid residue sequence selected from

i)
XₐX_{b}X_{c}AWX_{d}EIXₑX_{f}X_{g}PXₕLXᵢDXⱼQWPAFIEXₖLH
according to SEQ ID NO: 2

wherein, independently of each other,
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S; and
ii) an amino acid residue sequence having at least 93% identity to the sequence defined in i).

In a preferred embodiment Xₐ is Y. In a preferred embodiment X_{b} is L. In a preferred embodiment X_{c} is Q. In a preferred embodiment X_{f} is N. In a preferred embodiment Xₐ is Y, X_{b} is L, X_{c} is Q, and X_{f} is N.

In some embodiments the (BM) is selected from the group consisting of:

(SEQ ID NO: 11)
YLQAWYEIFNLPNLNDNQWPAFIESLH;
(SEQ ID NO: 12)
YLQAWYEIFNLPWLNDNQWPAFIESLH;
(SEQ ID NO: 13)
YLQAWYEIFNLPYLNDNQWPAFIESLH;
(SEQ ID NO: 14)
YLQAWYEIFQLPNLNDNQWPAFIESLH;
(SEQ ID NO: 15)
YLQAWWEIFNLPNLNDNQWPAFIESLH;
(SEQ ID NO: 16)
YLQAWFEIFNLPNLNDNQWPAFIESLH;
(SEQ ID NO: 17)
YLQAWHEIFNLPNLNDNQWPAFIESLH;
(SEQ ID NO: 18)
YLAAWYEIFNLPNLNDNQWPAFIESLH;
(SEQ ID NO: 19)
YLQAWYEIFNLPHLNDNQWPAFIESLH;
(SEQ ID NO: 20)
YLQAWYEIFNLPNLNDNQWPAFIEALH;
(SEQ ID NO: 21)
YLQAWYEIFNLPNLNDNQWPAFIEQLH;
(SEQ ID NO: 22)
YLQAWYEIFNLPNLQDNQWPAFIESLH;
(SEQ ID NO: 23)
YLQAWYEIFNLPNLNDQQWPAFIESLH;
(SEQ ID NO: 24)
YLQAWYEIFNLPNLNDDQWPAFIESLH;
(SEQ ID NO: 25)
YLQAWYEIFNLPNLNDEQWPAFIESLH;
(SEQ ID NO: 26)
YLQAWYEIFNLPNLNDSQWPAFIESLH;
(SEQ ID NO: 27)
YLQAWYEIFNLPNLNDTQWPAFIESLH;
(SEQ ID NO: 28)
YLQAWYEIFNLPNLNDHQWPAFIESLH;
(SEQ ID NO: 29)
YLQAWYEIFNLPNLNDAQWPAFIESLH;
(SEQ ID NO: 30)
YLQAWYEIFNLPNLNDLQWPAFIESLH;
(SEQ ID NO: 31)
YLQAWYEIFNLPNLTDNQWPAFIESLH;
(SEQ ID NO: 32)
YLQAWYEIFNLPNLTDEQWPAFIESLH;
(SEQ ID NO: 33)
YLQAWYEIFNLPNLTDQQWPAFIESLH;
(SEQ ID NO: 34)
YLQAWYEIFNLPNLSDNQWPAFIESLH;
(SEQ ID NO: 35)
YLQAWYEIFNLPNLNDGQWPAFIESLH;
(SEQ ID NO: 36)
YVQAWYEIFNLPNLNDNQWPAFIESLH;
(SEQ ID NO: 37)
YIQAWYEIFNLPNLNDNQWPAFIESLH;
(SEQ ID NO: 38)
YFQAWYEIFNLPNLNDNQWPAFIESLH;
(SEQ ID NO: 39)
YLQAWYEIFNLPQLNDNQWPAFIESLH;
(SEQ ID NO: 40)
YLQAWYEIFNLPNLQDQQWPAFIESLH;
(SEQ ID NO: 41)
YLQAWYEIFNLPHLNDQQWPAFIESLH;
(SEQ ID NO: 42)
YLQAWYEIFNLPNLDDAQWPAFIESLH;
(SEQ ID NO: 43)
YLQAWYEIFNLPNLTDAQWPAFIESLH;
(SEQ ID NO: 44)
YLQAWYEIFNLPNLDDQQWPAFIESLH;
(SEQ ID NO: 45)
YLQAWYEIFNLPHLNDAQWPAFIESLH;
(SEQ ID NO: 46)
YLQAWYEIFNLPNLNDYQWPAFIESLH;
(SEQ ID NO: 47)
YLQAWYEIFNLPHLTDQQWPAFIESLH;
(SEQ ID NO: 48)
YLQAWYEIFNLPNLTDYQWPAFIESLH;
(SEQ ID NO: 49)
YLQAWYEIFTLPHLTDQQWPAFIESLH;
(SEQ ID NO: 50)
YLQAWYEIFNLPCLNDNQWPAFIESLH;
(SEQ ID NO: 51)
YLQAWYEIFNLPCLNDQQWPAFIESLH;
(SEQ ID NO: 52)
YLQAWYEIFNLPCLTDSQWPAFIESLH.

In one aspect the compounds of the invention are characterized by reference to their binding motifs (BM) and the amino acid residues N- and C-terminally flanking the (BM), and can thus be described as hFGF23 antagonistic polypeptides, which comprise a binding motif (BM) as set out above, wherein said polypeptides comprise an amino acid residue sequence selected from

a)
X₁X₂X₃X₄X₅X₆X₇X₈-(BM)-X₃₆X₃₇PSQX₄₁X₄₂X₄₃LLX₄₆EARX₅₀LX₅₂X₅₃
X₅₄QX₅₆X₅₇X₅₈ according to SEQ ID NO: 3,

wherein (BM) consists of an amino acid residue sequence as defined in (i) above,
wherein, independently of each other,
X₁ is G, I, L, N or V;
X₂ is D, E or Q;
X₃ is D, E, G, N or Q;
X₄ is D, E, H, N, R or T;
X₅ is E, I, L, T or V;
X₆ is F, W or Y;
X₇ is A, G, H, I, L, N, P, Q, R, T or V;
X₈ is A, L or E;
X₃₆ is A, D or E;
X₃₇ is D or E;
X₄₁ is A, R or W;
X₄₂ is A, D or T;
X₄₃ is E, N, Q or S;
X₄₆ is A, E or K;
X₅₀ is Q or R;
X₅₂ is N or E;
X₅₃ is D, E, K or T;
X₅₄ is A or I;
X₅₆ is A or C;
X₅₇ is K or P;
X₅₈ is C, K or E; and
b) an amino acid residue sequence having at least 93% identity to a sequence defined in a).

In preferred embodiments, independently of each other,
X₁ is G, I, L or V;
X₂ is D or E;
X₃ is D or E;
X₄ is D or E;
X₅ is E;
X₆ is F;
X₇ is I or Q;
X₈ is A or L;
Xₐ is Y;
X_{b} is V;
X_{c} is Q;
X_{d} is Y;
Xₑ is F or Y;
X_{f} is N or Q;
X_{g} is L;
Xₕ is N;
Xᵢ is N or T;
Xⱼ is A, N or Q;
Xₖ is S;
X₃₆ is D or E;
X₃₇ is D or E;
X₄₁ is W;
X₄₂ is A;
X₄₃ is N;
X₄₆ is A or E;
X₅₀ is Q or R;
X₅₂ is N;
X₅₃ is E;
X₅₄ is A;
X₅₆ is A;
X₅₇ is P;
X₅₈ is K.

As the skilled person will realize, it is possible to make minor changes to the sequence of amino acids in a polypeptide without significantly affecting the function thereof. Thus, the present disclosure encompasses modified variants of the polypeptide, which retain their antagonistic characteristics. In this way, encompassed by the present disclosure is a hFGF23 antagonistic polypeptide comprising an amino acid sequence with 93% or greater identity, such as 95, 96, 97, 98 or 99% or greater identity, to the hFGF23 antagonistic polypeptide or (BM) as defined in above. For example, it is possible that an amino acid residue belonging to a certain functional grouping of amino acid residues (e g hydrophobic, hydrophilic, polar etc) could be exchanged for another amino acid residue from the same functional group.

In some embodiments a hFGF23 antagonistic polypeptide of the invention may comprise 50-70 amino acid residues. In preferred embodiments the hFGF23 antagonistic polypeptide may comprise 56-60 amino acid residues. In a more preferred embodiment the hFGF23 antagonistic polypeptide comprises 58 amino acid residues.

In preferred embodiments the hFGF23 antagonistic polypeptides comprise a protraction moiety. hFGF23 antagonistic polypeptides that comprises a protractor or protraction moiety may also be referred to as “hFGF23 antagonistic polypeptide derivatives”, the word derivative indicating that a protractor or protraction moiety is present.

Preferred compounds of the present invention are Chem. 89, Chem. 106, Chem. 107, Chem. 111, Chem. 197, Chem. 201, Chem. 204, Chem. 206, Chem. 207 and Chem. 208 (see table 4 below). Non-limiting examples are shown below. Chem. 204 is represented by the following formula:

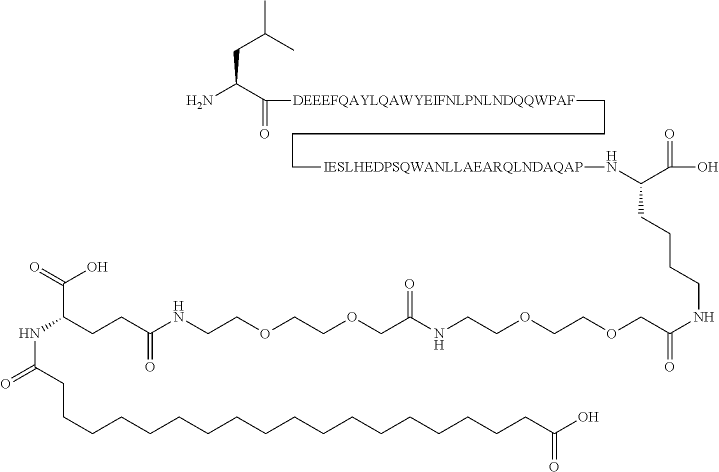

Chem. 206 is represented by the following formula:

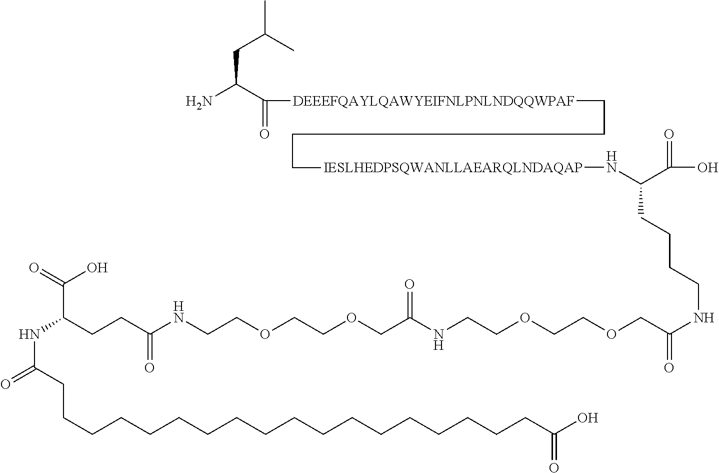

Chem. 207 is represented by the following formula:

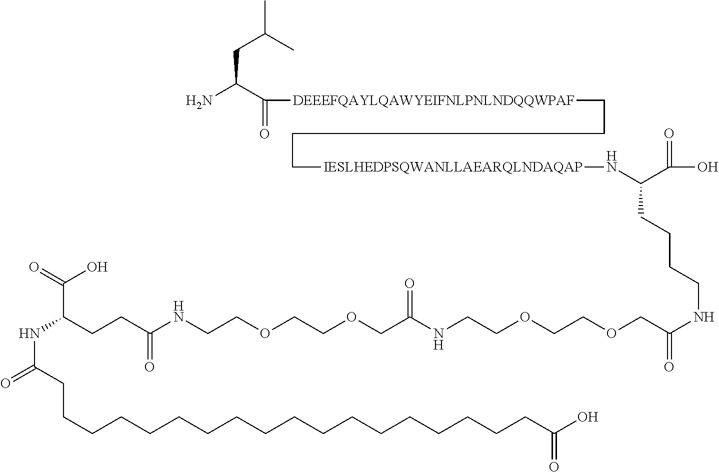

Chem. 208 is represented by the following formula:

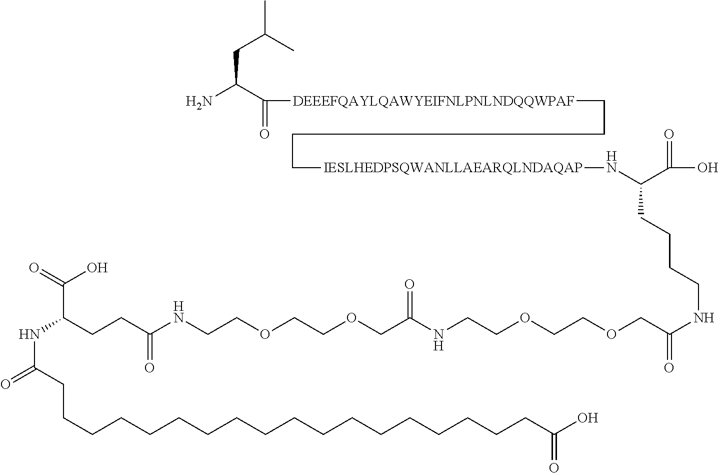

In one aspect the present invention relates to pharmaceutical formulations comprising the hFGF23 antagonistic polypeptides or hFGF23 antagonistic polypeptide derivatives of the invention and one or more pharmaceutically acceptable excipients.

In one aspect the present invention relates to medicinal use of the hFGF23 antagonistic polypeptides, hFGF23 antagonistic polypeptide derivatives and pharmaceutical formulations comprising the hFGF23 antagonistic polypeptides or derivatives thereof.

Such medicinal use includes use in the treatment of a hypophosphatemic disorder, such as—but not limited to—X-linked hypophosphatemic rickets (XLH).

In other aspects the present invention relates to a kit comprising the hFGF23 antagonistic polypeptide or polypeptide derivative, or said pharmaceutical composition;
a polynucleotide encoding the hFGF23 antagonistic polypeptide or polypeptide derivative, an expression vector encoding such polynucleotide;
a host cell which produces said hFGF23 antagonistic polypeptide or polypeptide derivatives;
as well as methods for producing said polypeptide or derivatives thereof comprising
a) culturing a host cell according to the preceding claim under conditions permissive of expression of the polypeptide encoded by said expression vector, b) isolating said polypeptide, and c) optionally attaching a protraction moiety to said isolated polypeptide.

The term “a” or “an” is intended to mean “one or more.”.

The term “about” is used herein to mean approximately, roughly, or around. When the term “about” is used in conjunction with a numerical range, it modifies that range by extending the boundaries above and below the numerical values set forth. In general, the term “about” can modify a numerical value above and below the stated value by 10%, up or down (higher or lower).

The term “comprise” and variations thereof such as “comprises” and “comprising,” when preceding the recitation of a step or an element, are intended to mean that the addition of further steps or elements is optional and not excluded.

As used throughout, “amino acids” or “amino acid residues” includes canonical amino acids (which are genetically encoded), and unnatural amino acids. Non-limiting examples of unnatural amino acids are Aib (α-aminoisobutyric acid), desamino histidine (alternative name 3-(imidazol-4-yl)propanoic acid, abbreviated Imp (imidazopropionyl) and the d-isomers of the canonical amino acids. All amino acid residues within the polypeptide for which the optical isomer is not stated is herein to be understood to mean the L-isomer, unless otherwise specified.

The term “binding affinity” is herein used as a measure of the strength of a non-covalent interaction between two molecules, e.g. a polypeptide and an antigen. The term “binding affinity” is used to describe monovalent interactions.

Binding affinity between two molecules, e.g. a hFGF23 binder, such as a hFGF23 antagonistic polypeptide as disclosed herein, and hFGF23, through a monovalent interaction may be quantified by determining the equilibrium dissociation constant (K_{D}). K_{D} can be determined by measurement of the kinetics of complex formation and dissociation, e.g. by the Surface Plasmon Resonance (SPR) method.

The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/Kₐ. Following the above definition, binding affinities associated with different molecular interactions, such as comparison of the binding affinity of different binders for a given antigen, may be compared by comparison of the K_{D} values for the individual binder/antigen complexes (such as the interaction between the compounds as disclosed herein and hFGF23).

The K_{D} of a hFGF23 antagonistic polypeptide for its target may be less than 100 μM such as less than 10 μM, such as less than 9 μM, such as less than 8 μM, such as less than 7 μM, such as less than 6 μM, such as less than 5 μM, such as less than 4 μM, such as less than 3 μM, such as less than 2 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 10 nM, such as less than 2 nM, such as less than 1 nM. Preferably the K_{D} is less than 0.1 μM, such as less than 10 nM, such as less than 2 nM, such as less than 1 nM. More preferably the K_{D} is less than 2 nM.

The term “binding motif” (BM) as used herein refers to the hFGF23 binding region of the hFGF23 antagonistic polypeptide. The (BM) comprises key binding residues enabling tight binding to hFGF23 and “forms part of” the three-helix bundle protein domain. For example, the (BM) may constitute two alpha helices with an interconnecting loop, within said three-helix bundle protein domain.

In some embodiments the (BM) comprises 25-28 amino acid residues. In a preferred embodiment the (BM) comprises 27 amino acid residues.

In some embodiments the (BM) sequence corresponds to the sequence from position 9 to position 35 (both positions included) in a sequence selected from the group consisting of SEQ ID NOs:11-52; or a sequence having 90% identity thereto, such as 91, 92, 93% or greater identity thereto, such as 94, 95, 96, 97, 98 or 99% identity to said (BM).

In some embodiments the (BM) is characterized in comprising amino acid residues which are either fixed or not fixed (variable). The non-fixed amino acid residues are identified herein by Xₓ, wherein the letter in subscript identifies the position in the (BM). N- and C-terminally the (BM) is flanked by non-(BM) amino acid residues and taken together the (BM) and non-(BM) residues make up a hFGF23 antagonistic polypeptide.

A “cross-species reactive” polypeptide binds to FGF23 from all indicated species (e.g. human, dog, pig and cynomolgus monkey) with comparable affinity, in particular with a K_{D} in the range of a factor of 100, such as within a range of a factor of 50, within a range of a factor of 20, or within a range of a factor of 10. Within a K_{D} range of a defined factor X means that the highest affinity for a particular listed species is not more than X-times higher than the lowest affinity measured for binding to a different listed species. A person skilled in the art will understand that any method for measuring affinity can be used to verify that a cross-species reactive polypeptide binds to the target antigen from all listed species within a given K_{D} factor range as described herein as long as the same conditions are applied to the K_{D} measurement for all listed species. Preferably, the K_{D} values are measured using SPR, in particular at 25° C.

The term “epitope”, as used herein, is defined in the context of a molecular interaction between an “antigen binding polypeptide”, such as a hFGF23 binder, such as the hFGF23 antagonistic polypeptides and derivatives thereof disclosed herein, and its corresponding antigen. Generally, “epitope” refers to the area or region on an antigen to which a binder binds, i.e. the area or region in physical contact with the binder. Physical contact may be defined using various criteria (e.g. a distance cut-off of 4-5 Å, such as 4, 4.5 or 5 Å; or solvent accessibility) for atoms in the binding molecule and antigen molecules.

hFGF23 may comprise a number of different epitopes, which may include, without limitation, (1) linear peptide epitopes (2) conformational epitopes which consist of one or more non-contiguous amino acids located near each other in the hFGF23 conformation. The epitope for a given binder/antigen pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen Deuterium eXchange Mass Spectrometry (HDX-MS), various competition binding methods and computational methods such as Alphafold 2; methods that are known in the art. In the context of an X-ray derived crystal structure defined by spatial coordinates of a complex between a binding molecule, e.g. a hFGF23 antagonistic polypeptide as disclosed herein, and its antigen, the term epitope is herein, unless otherwise specified or contradicted by context, specifically defined as hFGF23 residues characterized by having a heavy atom (i.e. a non-hydrogen atom) within a distance of 3.5 Å, from a heavy atom in the binding molecule.

Epitopes described at the amino acid level, e.g. determined from an X-ray structure, are said to be identical if they contain the same set of amino acid residues. Epitopes are said to overlap if at least one amino acid residue is shared by the epitopes. Epitopes are said to be separate (unique) if no amino acid residue is shared by the epitopes.

The epitope may be identified by routine methods. For example, the general location of an epitope may be determined by assessing the ability of hFGF23 antagonistic polypeptides to bind to different fragments or variants of hFGF23. The specific amino acids within hFGF23 that make contact with a binder (epitope) may also be determined using routine methods. For example, the binder and target molecule may be combined and the resulting complex may be crystallised. The crystal structure of the complex may be determined and used to identify specific sites of interaction between the antibody and its target. Epitope residues which are common to several different compounds may be regarded as ‘key’ epitope residues.

The term “hFGF23 antagonistic polypeptide” as used herein refers to a polypeptide which is capable of binding to hFGF23 according to SEQ ID NO:1 and capable of partly or completely blockings its biological activity, such as hFGF23-induced signalling activity, such as hFGF23-induced signalling activity of the FGFR1c-alpha-klotho complex. hFGF23 antagonistic polypeptides comprise a three-helix bundle protein domain being a variant of protein Z derived from the domain B of staphylococcal protein A.

In some embodiments the hFGF23 antagonistic polypeptide is characterized in comprising amino acid residues which are either fixed or not fixed. The non-fixed (variable) amino acid residues are identified herein by Xₓ, wherein the number/letter in subscript identifies the position in the hFGF23 antagonistic polypeptide.

The term “half-life” or “half-life in blood” as used herein refers to the time required for half the quantity of a substance administered to a patient to be metabolized or eliminated from the blood of the patient by normal biological processes.

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods.

In the present invention identity was determined using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5). The scores for each sequence in a pair are calculated as the number of matches in the aligned sequence, normalized by the sequence length and multiplied by 100.

The term “pharmaceutically acceptable excipient” means an excipient that is useful in preparing a pharmaceutical composition that is generally safe and non-toxic, and includes excipients that are acceptable for human pharmaceutical use. Such excipients can for example be solid, liquid or semisolid.

The term “polypeptide” as used herein includes oligopeptides and refers to a single chain of amino acids connected by one or more peptide bonds.

“Substitution” variants preferably involve the replacement of one or more amino acid(s) with the same number of amino acid(s). Substitutions may be, but are not limited to, conservative substitutions. For example, an amino acid may be substituted to an amino acid with similar biochemical properties, for example, a basic amino acid may be substituted to another basic amino acid (e.g. lysine to arginine), an acidic amino acid may be substituted to another acidic amino acid (e.g. glutamate to aspartate), a neutral amino acid may be substituted to another neutral amino acid (e.g. threonine to serine), a charged amino acid may be substituted to another charged amino acid (e.g. glutamate to lysine), a hydrophilic amino acid may be substituted to another hydrophilic amino acid (e.g. asparagine to glutamine), a hydrophobic amino acid may be substituted to another hydrophobic amino acid (e.g. alanine to valine), a polar amino acid may be substituted to another polar amino acid (e.g. serine to threonine), an aromatic amino acid may be substituted to another aromatic amino acid (e.g. phenylalanine to tryptophan) and an aliphatic amino acid may be substituted to another aliphatic amino acid (e.g. leucine to isoleucine). Such substitutions are referred to as “conservative” substitutions.

The present invention encompasses variants of the hFGF23 antagonistic polypeptides, as disclosed herein, which may comprise 1, 2, or 3 amino acid substitutions in the individual binding motif and hFGF23 antagonistic polypeptide sequences disclosed herein. In some embodiments the substitutions are conservative substitutions.

The term “treatment” and variations thereof, as used herein, refers to the medical therapy of any human subject in need thereof. The term includes administering a therapeutically effective amount of a polypeptide as disclosed herein sufficient to reduce or eliminate at least one symptom of the disorder in question. “Treatment”, however, need not be a cure. The timing and purpose of said treatment may vary from one individual to another, according to the status quo of the subject's health. Thus, said treatment may be prophylactic, disease modifying, palliative, symptomatic and/or curative. In terms of the present invention, prophylactic, disease modifying, palliative, symptomatic and/or curative treatments may represent separate aspects of the invention.

The term “protraction moiety” (PM) as used herein refers to a moiety capable of extending the half-life in blood of the compound to which it is attached. A protraction moiety comprises a “protractor” (P) and an optional “linker” (L_{P}). The term “protraction” thus refers to half-life extension and a protractor or protraction moiety thus serves the purpose of the extending half-life in blood of the hFGF23 antagonistic polypeptides disclosed herein. The terms “protracted” and “half-life extended” are used interchangeably and generally refer to an extension of the blood plasma half-life of the compounds as disclosed herein.

The protraction moiety is preferably covalently attached to the side chain of a surface exposed lysine or a cysteine residue in the polypeptide after the polypeptide as such has been produced (e.g. by synthesis or recombinant expression), preferably via the epsilon-amino group. Herein, the residue making up the point of attachment for such attachment is generally referred to as “R1” (and in case of more than one protraction moiety “R2”, “R3” asf.).

In view of the disclosure herein the skilled person will be able to identify other surface exposed residue(s) suitable for attachment.

A protraction moiety may consist of one protractor (and no linker (“L_{P}”)).

A protraction moiety may comprise one linker (“L_{P}”) and one protractor (“P”).

A protraction moiety may comprise one linker and two or more protractors (“P”).

When the linker (“L_{P}”) is present, the protraction moiety attaches to the hFGF23 antagonistic polypeptide backbone via L_{P}. When the linker (“L_{P}”) is absent, “P” attaches directly to the polypeptide backbone.

hFGF23 antagonistic polypeptide derivatives—i.e. a hFGF23 antagonistic polypeptide to which a protraction moiety is attached—as disclosed herein may comprise a single cysteine or lysine residue, to which a single protraction moiety is attached.

The hFGF23 antagonistic polypeptide derivative as disclosed herein may comprise two lysine residues and two protraction moieties.

The hFGF23 antagonistic polypeptide derivative as disclosed herein may comprise two lysine residues and two identical protraction moieties.

The hFGF23 antagonistic polypeptide derivative as disclosed herein may comprise two lysine residues and two non-identical protraction moieties.

The hFGF23 antagonistic polypeptide derivative as disclosed herein may comprise three lysine residues and three protraction moieties.

The hFGF23 antagonistic polypeptide derivative as disclosed herein may comprise three lysine residues and three identical protraction moieties.

The hFGF23 antagonistic polypeptide derivative as disclosed herein may comprise three lysine residues and three non-identical protraction moieties (for example, the first and second protraction moieties may be identical and the third protraction moiety different form said first and second moieties).

The hFGF23 antagonistic polypeptide derivative as disclosed herein may comprise two cysteine residues and two protraction moieties.

The hFGF23 antagonistic polypeptide derivative as disclosed herein may comprise two cysteine residues and two identical protraction moieties.

The hFGF23 antagonistic polypeptide derivative as disclosed herein may comprise two cysteine residues and two non-identical protraction moieties.

The hFGF23 antagonistic polypeptide derivative as disclosed herein may comprise three cysteine residues and three protraction moieties.

The hFGF23 antagonistic polypeptide derivative as disclosed herein may comprise three cysteine residues and three identical protraction moieties.

The hFGF23 antagonistic polypeptide derivative as disclosed herein may comprise three cysteine residues and three non-identical protraction moieties.

Where the hFGF23 antagonistic polypeptide derivative comprises two or three protraction moieties, the protracting moieties are preferably similar, more preferably substantially identical, or, most preferably, identical.

Herein, hFGF23 antagonistic polypeptides comprising protraction moiety(ies) are in some instances referred as polypeptides comprising a protraction moiety or simply as polypeptide “derivatives”.

The protraction moiety may be capable of non-covalently binding to albumin, thereby promoting the circulation of the hFGF23 antagonistic polypeptide derivative in the blood stream and prolonging its half-life. Thus, in one embodiment the protraction moiety is an albumin binding moiety.

The protractor may comprise an acyl group. The protractor may comprise a fatty acyl group. The acyl group may be branched or unbranched. The acyl group may be saturated or unsaturated.

The protractor may comprise a distal carboxylic acid group.

The protractor may comprise at least two acidic groups, wherein one acidic group is attached terminally.

The protractor may comprise a fatty acid group.

The protractor may comprise a fatty acid group and an amide group.

The protractor may comprise a distal carboxylic acid group and an amide group.

The protractor may comprise an alkyl group.

The protractor may comprise an aryl group.

The protractor may comprise a tetrazole group.

The protractor may comprise a sulfonic acid group.

The protractor may comprise a phenoxy group.

The protractor may comprise a benzoic acid group.

The protractor may comprise 8-30 carbon atoms. The protractor may comprise 8, 9, 10, 11, 12, 13, 14, 15, 16, 17, 18, 19, 20, 21, 22, 23, 24, 25, 26, 27, 28, 29 or 30 carbon atoms.

The protractor may comprise 6-30 consecutive —CH₂— groups. The protractor may comprise a carbon chain comprising at least 6, 7, 8, 9, 10, 11, 12, 13, 14, 15, 16, 17, 18, 19, 20, 21, 22, 23, 24, 25, 26, 27, 28, 29 or 30 consecutive —CH₂— groups.

The protractor may comprise 12-26 carbon atoms. The protractor may comprise 12, 13, 14, 15, 16, 17, 18, 19, 20, 21, 22, 23, 24, 25 or 26 carbon atoms.

The protractor may comprise 10-26 consecutive —CH₂— groups. The protractor may comprise a carbon chain comprising 10, 11, 12, 13, 14, 15, 16, 17, 18, 19, 20, 21, 22, 23, 24, 25 or 26 consecutive —CH₂— groups.

The protractor may comprise 16-22 carbon atoms. The protractor may comprise 16, 17, 18, 19, 20, 21 or 22 carbon atoms.

The protractor may comprise 14-20 consecutive —CH₂— groups. The protractor may comprise a carbon chain comprising 14, 15, 16, 17, 18, 19 or 20 consecutive —CH₂-groups.

The protractor may comprise 16-22 consecutive carbon atoms and 14-20 consecutive —CH₂— groups.

The protractor may comprise 16 consecutive carbon atoms and 14 consecutive —CH₂-groups.

The protractor may comprise 18 consecutive carbon atoms and 16 consecutive —CH₂-groups.

The protractor may comprise 20 consecutive carbon atoms and 18 consecutive —CH₂-groups.

The protractor may comprise 22 consecutive carbon atoms and 20 consecutive —CH₂-groups.

The protractor may comprise a group defined by:
Chem. 1: HOOC—(CH₂)ₙ—CO—* wherein n is an integer in the range of 8-30, which may also be referred to as a C(n+2) diacid, or as Chem. 1:

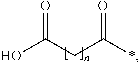

wherein n is an integer in the range of 8-30.

The protractor may comprise an oligopeptide. The protractor oligopeptide may be 10 to 40 amino acids, such as 10 to 30 amino acids, such as 15 to 25 amino acids, and, preferably, 20 amino acids. The protractor oligopeptide sequence composition may for example be GlnArgLeuMetGluAsplleCysLeuProArgTrpGlyCysLeuTrpGluAspAspPhe (single amino acid notation QRLMEDICLPRWGCLWEDDF (Chem. 1f, SEQ ID NO:9) (see also WO01/45746 Å2).

The protractor may be attached to the hFGF23 antagonistic polypeptide via an amino acid linker L_{P}.

In one embodiment L_{P} may join the protractor to the side chain of a lysine or cysteine residue in the hFGF23 antagonistic polypeptide backbone.

In one embodiment L_{P} may join the protractor to the N- or C-terminal residue in the hFGF23 antagonistic polypeptide backbone.

In one embodiment L_{P} may join the protractor to residue 46K, 53K or 57K in the hFGF23 antagonistic polypeptide.

The hFGF23 antagonistic polypeptide derivative may comprise two protraction moieties, each of which comprises 14, 15, 16, 17, 18, 19 or 20 carbon atoms.

The hFGF23 antagonistic polypeptide derivative may comprise two protraction moieties, wherein each protractor comprises 12, 13, 14, 15, 16, 17 or 18 consecutive —CH₂— groups, such protractors are also designated C12, C13, C14, C15, C16, C17 or C18 protractors.

In some embodiments the protractor comprises acidic groups, such as a carboxylic acid group flanking the consecutive —CH2-groups in each end (diacids).

The hFGF23 antagonistic polypeptide derivative may comprise two C14 diacids, two C16 diacids or two C18 diacids.

The hFGF23 antagonistic polypeptide derivative may comprise three protraction moieties, each of which comprises a protractor comprising 12, 13, 14, 15, 16, 17, 18, 19 or 20 carbon atoms. The hFGF23 antagonistic polypeptide derivative may comprise three protraction moieties, wherein the protractor comprises 10, 11, 12 13, 14, 15, 16, 17 or 18 consecutive —CH₂— groups.

In some embodiments the protractor comprises a C16, C17, C18, C19, C20, C21 or C22 fatty acid, such as a C16, C17, C18, C19 or C20 fatty diacid.

In a preferred embodiment the hFGF23 antagonistic polypeptide derivative comprises a diacid protractor comprising 20 consecutive —CH₂— groups (a C20 diacid protractor).

In a preferred embodiment said 020 diacid protractor is Chem. 1c in Table 1 below.

In some embodiments a hFGF23 antagonistic polypeptide derivative as disclosed herein may comprise a protractor which is selected from any one of those depicted in Table 1.

TABLE 1
Examples of protractors (“P”)

| Protractor | |
|---|---|
| ID | Structure |
| C16 diacid (Chem. 1a) | 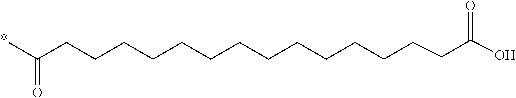 [SMILES: CC(=O)CCCCCCCCCCCCCCC(=O)O] |
| C18 diacid (Chem. 1b) | 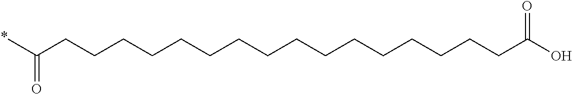 [SMILES: CC(=O)CCCCCCCCCCCCCCCCC(=O)O] |
| C20 diacid (Chem. 1c) | 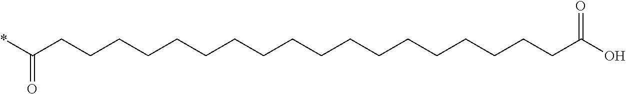 [SMILES: *C(=O)CCCCCCCCCCCCCCCCCCC(=O)O] |
| C17 tetrazole (Chem. 1d) | 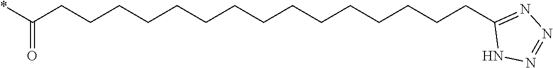 [SMILES: *C(=O)CCCCCCCCCCCCCCCc1nnn[nH]1] |
| C18 tetrazole (Chem. 1e) | 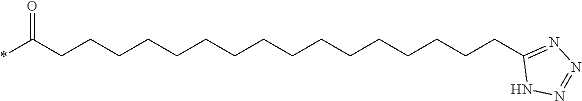 [SMILES: *C(=O)CCCCCCCCCCCCCCCCc1nnn[nH]1] |
| Albumin- binder oligopeptide (Chem. 1f, SEQ ID NO: 9) | 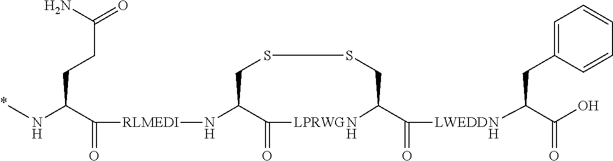 |
| | 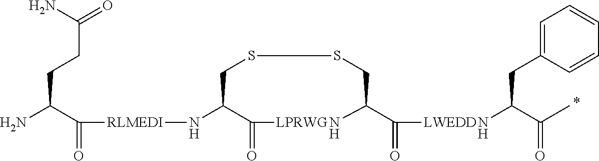 |

wherein an asterix (*) designates the point of attachment to a L_{P} linker element, or an amino acid residue in the hFGF23 antagonistic polypeptide.

The term “protraction moiety” as used herein refers to a moiety having half-life extending properties and comprising a “protractor” (“P”) and an optional “linker” (“L_{P}”), said linker—if present—linking the protractor to the hFGF23 antagonistic polypeptide The L_{P} linker—when present—may comprise Ado, Aeep or Aeeep, sulfonamide, Trx, ε-Lys, Ahx, Glu, γGlu, Gly, Ser, Ala and/or Thr.

The L_{P} linker may for example comprise at least a moiety which may be represented by the chemical formulas below wherein an asterix (*) designates the point of attachment to a further L_{P} linker element, a protractor, or an amino acid residue in the hFGF23 antagonistic polypeptide. The skilled person will understand that the first and second * in the individual chemical formulas below will not designate attachment points on the same type of element, i.e. the linkers according to the below formulas are not intended to link two hFGF23 antagonistic polypeptides nor two protractors.

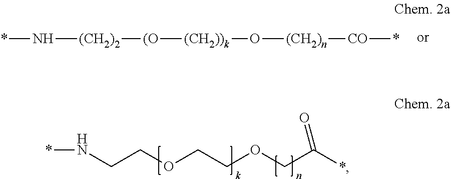

wherein k is an integer in the range of 1-5, and n is an integer in the range of 1-5.

When k=1 and n=1, the linker element may be designated Ado, or 8-amino-3,6-dioxaoctanoyl, which may be represented by the following chemical formula:

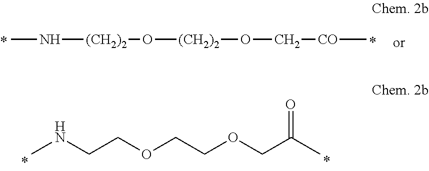

When k=1 and n=2, the linker element may be designated Aeep, which may be represented by the following chemical formula:

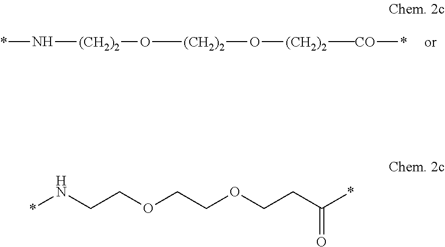

When k=2 and n=2, the linker element may be designated Aeeep, which may be represented by the following chemical formula:

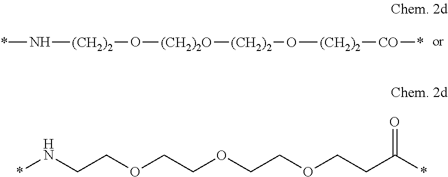

The optional linker (L_{P}) may comprise a sulfonamide-C4 moiety. A sulfonamide-C4 group is a sulfonamide group attached to a 4-butanoyl group, having the following chemical formula:

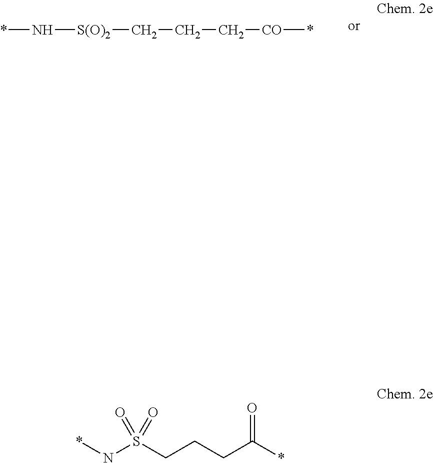

The optional linker L_{P} may comprise Trx. Trx is also referred to as Tranexamic acid, trans-4-(aminomethyl)cyclohexanecarboxylic acid, having the following chemical formula:

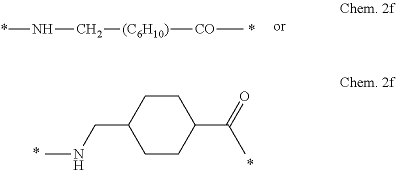

The linker L_{P} may comprise epsilon-lysine (c-Lys).

The linker L_{P} may comprise lysine (Lys).

The linker L_{P} may comprise Ahx. Ahx is also referred to as Aminocaproic acid, 6-aminohexanoic acid and is defined by

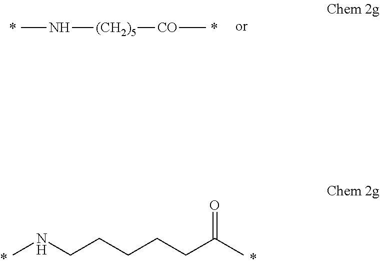

The linker L_{P} may comprise a Glu di-radical, such as

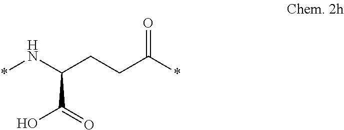 [SMILES: *N[C@@H](CCC(*)=O)C(=O)O]

wherein the Glu di-radical may be included p times, where p is an integer in the range of 1-3.

Chem. 2 h may also be referred to as gamma-Glu, or briefly γGlu, since it is the gamma carboxy group of the amino acid glutamic acid (E) which is here used for connection to the epsilon-amino group of lysine. As explained above, the other linker element may, for example, be another Glu residue, or an Ado molecule. The amino group of Glu in turn forms an amide bond with the carboxy group of the protractor moiety, or with the carboxy group of, e.g., an Ado molecule, if present, or with the gamma-carboxy group of, e.g., another Glu, if present, such as:

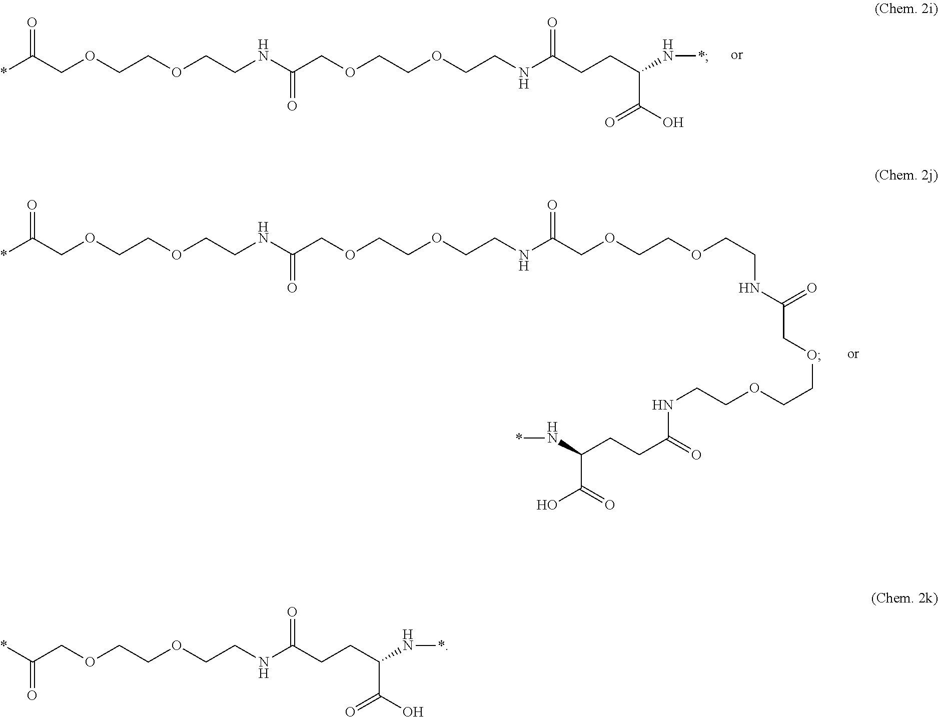

In preferred embodiments the protractor, exemplified by Chems. 1a-1f, is attached to the polypeptide backbone using a linker (L_{P}) selected from Table 2 below wherein “R1” represents the site of attachment in the hFGF23 antagonistic polypeptide and “P” represents the protractor.

In preferred embodiments the protractor Chem. 1a-1f is attached to the polypeptide backbone using the linker (L_{P}) designated L1 (Chem. 2i) in Table 2 below.

In a preferred embodiment the site of attachment R1 is on the epsilon-amino group of the polypeptide backbone.

In one such embodiment the site of attachment R1 is on the epsilon-amino group of the C-terminal residue lysine of the polypeptide backbone.

In another such embodiment the site of attachment R1 is on the epsilon-amino group of lysine in position 46, 53 or 57 of the hFGF23 antagonistic polypeptide.

Thus, in preferred embodiments the residue in position 46, 53 and/or 57 is lysine (K).

In a preferred embodiment the residue in position 58 is lysine (K).

Based on the disclosure herein, the skilled person will be able to determine the optimal L_{P} linker for use in a specific hFGF23 antagonistic polypeptide derivative as disclosed herein, optionally after some limited routine experiments.

TABLE 2
Examples of preferred protraction moiety linkers (“L_{P}”)

| Linker | |
|---|---|
| (L_{P}) | |
| ID | Structure |
| L1 (Chem. 2i) | 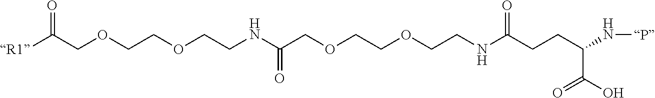 |
| L2 (Chem. 2j) | 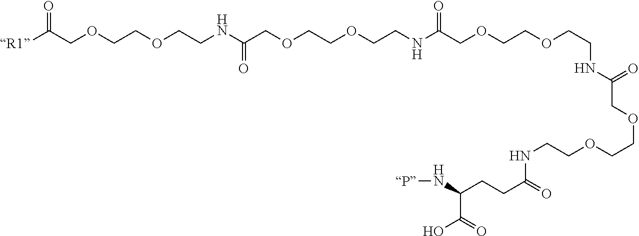 |
| L3 (Chem. 2k) | 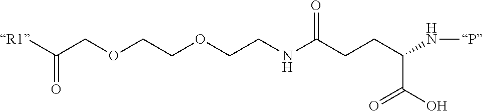 |

The compounds as disclosed herein may be produced by means of solid-phase peptide synthesis as described in the Examples section below. Fmoc-Lys(Mtt)-OH, which is installed at the site of protraction moiety attachment, allows for selective removal of the Mtt-group followed by installation of the protraction moiety during peptide synthesis. Global deprotection of resin bound peptides was performed after synthesis followed by reverse-phase HPLC purification.

The compounds as disclosed herein may be produced by means of recombinant nucleic acid techniques. In general, a cloned wild-type compounds as disclosed herein nucleic acid sequence is modified to encode the desired protein. This modified sequence is then inserted into an expression vector, which is in turn transformed or transfected into host cells.

The nucleic acid construct encoding the compounds as disclosed herein may suitably be of genomic, cDNA or synthetic origin. Amino acid sequence alterations are accomplished by modification of the genetic code by well-known techniques.

The DNA sequences encoding the compounds as disclosed herein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the compounds as disclosed herein is operably linked to additional segments required for transcription of the DNA. The term, “operably linked” indicates that the segments are arranged so that they function in concert for their intended purposes, e.g. transcription initiates in a promoter and proceeds through the DNA sequence coding for the polypeptide until it terminates within a terminator.

Thus, expression vectors for use in expressing compounds as disclosed herein will comprise a promoter capable of initiating and directing the transcription of a cloned gene or cDNA. The promoter may be any DNA sequence, which shows transcriptional activity in the host cell of choice and may be derived from genes encoding proteins either homologous or heterologous to the host cell.

Additionally, expression vectors for use of expression of compounds as disclosed herein will also comprise a terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Expression of compounds as disclosed herein can be aimed for either intracellular expression in the cytosol of the host cell or be directed into the secretory pathway for extracellular expression into the growth medium.

Intracellular expression is the default pathway and requires an expression vector with a DNA sequence comprising a promoter followed by the DNA sequence encoding the compounds as disclosed herein polypeptide followed by a terminator.

To direct the compounds as disclosed herein into the secretory pathway of the host cells, a secretory signal sequence (also known as signal peptide or a pre sequence) is needed as an N-terminal extension of the compounds as disclosed herein. A DNA sequence encoding the signal peptide is joined to the 5′ end of the DNA sequence encoding the compounds as disclosed herein in the correct reading frame. The signal peptide may be that normally associated with the protein or may be from a gene encoding another secreted protein.

The procedures used to ligate the DNA sequences coding for the compounds as disclosed herein, the promoter, the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, New York, 1989).

The host cell into which the DNA sequences encoding compounds as disclosed herein is introduced may be any cell that is capable of expressing compounds as disclosed herein either intracellularly or extracellularly. If posttranslational modifications are needed, suitable host cells include yeast, fungi, insects and higher eukaryotic cells such as mammalian cells.

The protraction moiety can be attached to polypeptide backbone as follows.

To a solution of a polypeptide backbone (10-40 mg/mL, 0.25 M Na₂CO₃(aq)) is added N-methylpyrrolidinone (0.25× volume of peptide solution) under stirring. To obtain selective reaction conditions for the epsilon-amino group of lysine, pH is increased by slow addition of aqueous sodium hydroxide until pH reaches 10.5-11.

The acylation reagent (2-4 molar equivalents compared to the polypeptide backbone) is dissolved in N-methylpyrrolidinone, and added to the stirred peptide solution over 2-60 min while continuously adjusting pH to 10.5-11 by addition of aqueous sodium hydroxide. The final reaction mixture is stirred at room temperature at constant pH until the acylation reagent is consumed (0-4 h). The reaction mixture is neutralised by dropwise addition of acetic acid and purified by reverse-phase HPLC.

Thus, the protraction moiety can be attached to the polypeptide as described above. In other embodiments of the invention the protraction moiety can be part of the polypeptide backbone, i.e. a fusion polypeptide relying on attachment via a peptidic amide bond. Fusion polypeptides can be obtained synthetically or by recombinant expression by methods known to a person skilled in the art.

The hFGF23 antagonistic polypeptide derivatives as disclosed herein may be prepared in pharmaceutical compositions. In some embodiments such composition comprises at least one pharmaceutically acceptable excipient.

The excipients may serve various purposes, e.g. as a carrier, vehicle, filler, binder, lubricant, glidant, disintegrant, flow control agent, crystallization inhibitors, solubilizer, stabilizer, colouring agent, flavouring agent, surfactant, emulsifier, delivery agent, hydrotrope or combinations thereof and/or to improve administration, and/or absorption of the active pharmaceutical ingredient(s).

The amount of each excipient used may vary within ranges conventional in the art. Techniques and excipients which may be used are described in e.g Remington: the Science and Practice of Pharmacy, 22^{nd} edition, Remington and Allen, Eds., Pharmaceutical Press (2013).

The compositions containing the compounds as disclosed herein can be administered for prophylactic and/or in some embodiments on-demand treatments.

The compositions are typically administered to a subject already suffering from a disease, such as the indications described below, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease as well as the weight and general state of the subject.

A compound as disclosed herein, such as a hFGF23 antagonistic polypeptide, such as a hFGF23 antagonistic polypeptide derivative, may be administered parenterally, such as intravenously, such as intramuscularly, such as subcutaneously in an appropriate pharmaceutical composition.

In some embodiments the dose of the compound to be delivered e.g. by subcutaneous administration may be from about 0.1 mg to 500 mg of the compound per day, preferably from about 0.5 mg to 150 mg per day, depending on the severity of the condition.

A suitable dose may also be adjusted for a particular compound based on the properties of that compound, including its in vivo half-life or mean residence time in blood and its biological activity.

In one embodiment the present invention relates to an injection device with content of said composition.

In one embodiment the compounds can be used in low burden administration devices that are amenable to self or caregiver drug delivery at home.

In one embodiment the hFGF23 antagonistic polypeptide or polypeptide derivative is a cross-species reactive polypeptide or polypeptide derivative.

In one embodiment the hFGF23 antagonistic polypeptide or polypeptide derivative provides an IC₅₀ value of less than 1 nM in an inhibitory potency assay as disclosed herein.

In one embodiment the hFGF23 antagonistic polypeptide or polypeptide derivative provides a % residual activity at 1 nM below 50% in an inhibitory potency assay as disclosed herein.

In one embodiment the compounds of the present invention provide improved phosphate levels in the body as compared to compounds of the art, such as burosumab.

### FURTHER EMBODIMENTS

1. A hFGF23 antagonistic polypeptide, which is capable of binding an epitope comprising the amino acid residues selected from the group consisting of:
   a) R76, T86, R91 and R114;
   b) M74, R76, T86, R91, F108, P110, R114, L166, I167 and N170;
   c) R76, T86, R91, Y93, Y107, R114, F169 and N170;
   d) W36, R76, T86, R91, Y107, R114, 1167, F169 and N170; and
   e) R76, T86, R91, R114, L166 and F169;
on hFGF23 according to SEQ ID NO:1 as determined at 3.5 Å.
   2. The hFGF23 antagonistic polypeptide according to embodiment 1, which is capable of binding hFGF23 according to SEQ ID NO:1, comprising a binding motif (BM) which consists of an amino acid residue sequence selected from

i)
XₐX_{b}X_{c}AWX_{d}EIXₑX_{f}X_{g}PXₕLXᵢDXⱼQWPAFIEXₖLH
according to SEQ ID NO: 2

wherein, independently of each other,
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S; and
ii) an amino acid residue sequence having at least 93% identity to the sequence defined in i).
3. A hFGF23 antagonistic polypeptide, which is capable of binding hFGF23 according to SEQ ID NO:1, comprising a binding motif (BM) which consists of an amino acid residue sequence selected from

i)
XₐX_{b}X_{c}AWX_{d}EIXₑX_{f}X_{g}PXₕLXᵢDXⱼQWPAFIEXₖLH
according to SEQ ID NO: 2

wherein, independently of each other,
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S; and
ii) an amino acid residue sequence having at least 93% identity to the sequence defined in i).
4. The hFGF23 antagonistic polypeptide according to embodiment 2 or 3, wherein the (BM) is selected from the group consisting of:
SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51 and SEQ ID NO:52; optionally comprising 1, 2, or 3 substitution(s), wherein said substitution(s) can take place in any one of positions 1, 2, 3, 6, 9, 10, 11, 13, 15, 17 and/or 25 in the selected (BM) sequence.
5. The hFGF23 antagonistic polypeptide according to embodiment 4, wherein the substitution(s) is/are conservative substitution(s).
6. The hFGF23 antagonistic polypeptide according to any one of the preceding embodiments, which comprises a binding motif (BM), wherein said polypeptide comprises an amino acid residue sequence selected from

a)
X₁X₂X₃X₄X₅X₆X₇X₈-(BM)-X₃₆X₃₇PSQX₄₁X₄₂
X₄₃LLX₄₆EARX₅₀LX₅₂X₅₃X₅₄QX₅₆X₅₇X₅₈
according to SEQ ID NO: 3,

wherein (BM) consists of an amino acid residue sequence as defined in embodiment 2(i),
wherein, independently of each other,
   X₁ is G, I, L, N or V;
   X₂ is D, E or Q;
   X₃ is D, E, G, N or Q;
   X₄ is D, E, H, N, R or T;
   X₅ is E, I, L, T or V;
   X₆ is F, W or Y;
   X₇ is A, G, H, I, L, N, P, Q, R, T or V;
   X₈ is A, L or E;
   X₃₆ is A, D or E;
   X₃₇ is D or E;
   X₄₁ is A, R or W;
   X₄₂ is A, D or T;
   X₄₃ is E, N, Q or S;
   X₄₆ is A, E or K;
   X₅₀ is Q or R;
   X₅₂ is N or E;
   X₅₃ is D, E, K or T;
   X₅₄ is A or I;
   X₅₆ is A or C;
   X₅₇ is K or P;
   X₅₈ is C, K or E; and
b) an amino acid residue sequence having at least 93% identity to the sequence defined in a).
7. A hFGF23 antagonistic polypeptide, which is capable of binding hFGF23 according to SEQ ID NO:1, wherein said polypeptide comprises an amino acid residue sequence selected from

a)
X₁X₂X₃X₄X₅X₆X₇X₈XₐX_{b}X_{c}WX_{d}EIXₑX_{f}X_{g}PXₕLXᵢDXⱼQWPA
FIEXₖLHX₃₆X₃₇PSQX₄₁X₄₂X₄₃LLX₄₆EARX₅₀LX₅₂X₅₃X₅₄
QX₅₆X₅₇X₅₈
according to SEQ ID NO: 3

wherein, independently of each other,
X₁ is G, I, L, N or V;
X₂ is D, E or Q;
X₃ is D, E, G, N or Q;
X₄ is D, E, H, N, R or T;
X₅ is E, I, L, T or V;
X₆ is F, W or Y;
X₇ is A, G, H, I, L, N, P, Q, R, T or V;
X₈ is A, L or E;
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S;
X₃₆ is A, D or E;
X₃₇ is D or E;
X₄₁ is A, R or W;
X₄₂ is A, D or T;
X₄₃ is E, N, Q or S;
X₄₆ is A, E or K;
X₅₀ is Q or R;
X₅₂ is N or E;
X₅₃ is D, E, K or T;
X₅₄ is A or I;
X₅₆ is A or C;
X₅₇ is K or P;
X₅₈ is C, K or E; and
b) an amino acid residue sequence having at least 93% identity to a sequence defined in a).
8. A hFGF23 antagonistic polypeptide, which is capable of binding hFGF23 according to SEQ ID NO:1, comprising an amino acid residue sequence

X₁X₂X₃X₄X₅X₆X₇X₈XₐX_{b}X_{c}WX_{d}EIXₑX_{f}X_{g}PXₕLXᵢDXⱼQWPA
FIEXₖLHX₃₆X₃₇PSQX₄₁X₄₂X₄₃LLX₄₆EARX₅₀LX₅₂X₅₃X₅₄
QX₅₆X₅₇X₅₈
according to SEQ ID NO: 3

wherein, independently of each other,
X₁ is G, I, L, N or V;
X₂ is D, E or Q;
X₃ is D, E, G, N or Q;
X₄ is D, E, H, N, R or T;
X₅ is E, I, L, T or V;
X₆ is F, W or Y;
X₇ is A, G, H, I, L, N, P, Q, R, T or V;
X₈ is A, L or E;
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S;
X₃₆ is A, D or E;
X₃₇ is D or E;
X₄₁ is A, R or W;
X₄₂ is A, D or T;
X₄₃ is E, N, Q or S;
X₄₆ is A, E or K;
X₅₀ is Q or R;
X₅₂ is N or E;
X₅₃ is D, E, K or T;
X₅₄ is A or I;
X₅₅ is A or C;
X₅₇ is K or P;
X₅₈ is C, K or E;
optionally comprising 1, 2, or 3 substitution(s), wherein said substitution(s) can take place in any one of the above non-fixed positions.
9. The hFGF23 antagonistic polypeptide according to embodiment 8, wherein the substitution(s) is/are conservative substitution(s).
10. The hFGF23 antagonistic polypeptide according to any one of embodiments 6-9 wherein, independently of each other,
   X₁ is G, I, L or V;
   X₂ is D or E;
   X₃ is D or E;
   X₄ is D or E;
   X₅ is E;
   X₆ is F;
   X₇ is I or Q;
   X₈ is A or L;
   Xₐ is Y;
   X_{b} is V;
   X_{c} is Q;
   X_{d} is Y;
   Xₑ is F or Y;
   X_{f} is N or Q;
   X_{g} is L;
   Xₕ is N;
   Xᵢ is N or T;
   Xⱼ is A, N or Q;
   Xₖ is S;
   X₃₆ is D or E;
   X₃₇ is D or E;
   X₄₁ is W;
   X₄₂ is A;
   X₄₃ is N;
   X₄₆ is A or E;
   X₅₀ is Q or R;
   X₅₂ is N;
   X₅₃ is E;
   X₅₄ is A;
   X₅₆ is A;
   X₅₇ is P;
   X₅₈ is K.
11. A hFGF23 antagonistic polypeptide, which is capable of binding hFGF23 according to SEQ ID NO:1, having at least 93% identity to a hFGF23 antagonistic polypeptide selected from the group consisting of:
   Chem. 197 according to SEQ ID NO:168;
   Chem. 201 according to SEQ ID NO:176;
   Chem. 204 according to SEQ ID NO:234;
   Chem. 206 according to SEQ ID NO:236;
   Chem. 207 according to SEQ ID NO:237; and
   Chem. 208 according to SEQ ID NO:238.
12. The hFGF23 antagonistic polypeptide according to embodiment 11, having at least 94%, at least 95%, at least 96%, at least 97%, at least 98%, or at least 99% identity to a hFGF23 antagonistic polypeptide selected from the group consisting of:
   Chem. 197 according to SEQ ID NO:168;
   Chem. 201 according to SEQ ID NO:176;
   Chem. 204 according to SEQ ID NO:234;
   Chem. 206 according to SEQ ID NO:236;
   Chem. 207 according to SEQ ID NO:237; and
   Chem. 208 according to SEQ ID NO:238.
13. The hFGF23 antagonistic polypeptide according to embodiment 11 or 12 which is capable of binding an epitope comprising the amino acid residues selected from the group consisting of:
   a) R76, T86, R91 and R114;
   b) M74, R76, T86, R91, F108, P110, R114, L166, I167 and N170;
   c) R76, T86, R91, Y93, Y107, R114, F169 and N170;
   d) W36, R76, T86, R91, Y107, R114, 1167, F169 and N170; and
   e) R76, T86, R91, R114, L166 and F169;
   on hFGF23 according to SEQ ID NO:1 as determined at 3.5 Å.
14. The hFGF23 antagonistic polypeptide according to any one of embodiments 2-6, wherein said binding motif (BM) forms part of a three-helix bundle protein domain, said three-helix bundle protein domain being a variant of protein Z, which is derived from domain B of staphylococcal Protein A.
15. The hFGF23 antagonistic polypeptide according to any one of the preceding embodiments wherein the polypeptide comprises 58 amino acid residues.
16. The hFGF23 antagonistic polypeptide according to any one of the preceding embodiments, wherein the polypeptide is selected from the group consisting of:
   Chem. 89;
   Chem. 106;
   Chem. 107;
   Chem. 111;
   Chem. 197;
   Chem. 201;
   Chem. 204;
   Chem. 206;
   Chem. 207; and
   Chem. 208.
17. The hFGF23 antagonistic polypeptide according to any one of the preceding embodiments wherein the polypeptide is capable of binding hFGF23 with a K_{D} value of less than 100 μM such as less than 10 μM, such as less than 9 μM, such as less than 8 μM, such as less than 7 μM, such as less than 6 μM, such as less than 5 μM, such as less than 4 μM, such as less than 3 μM, such as less than 2 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 10 nM, such as less than 1 nM, preferably less than 0.1 μM, such as less than 10 nM, such as less than 2 nM, such as less than 1 nM.
18. The hFGF23 antagonistic polypeptide according to any one of the preceding embodiments wherein the polypeptide comprises a three-helix bundle protein domain being a variant of protein Z which is derived from domain B of staphylococcal Protein A.
19. The hFGF23 antagonistic polypeptide according to any one of the preceding embodiments wherein the polypeptide is a three-helix bundle protein domain being a variant of protein Z which is derived from domain B of staphylococcal Protein A.
20. The hFGF23 antagonistic polypeptide according to any one of the preceding embodiments wherein the polypeptide is an AFFIBODY® or derived from an AFFIBODY®.
21. The hFGF23 antagonistic polypeptide according to any one of the preceding embodiments wherein said polypeptide comprises a protraction moiety.
22. The hFGF23 antagonistic polypeptide according to any one of the preceding embodiments wherein the protraction moiety is covalently attached to an amino acid residue side chain group on said polypeptide.
23. The hFGF23 antagonistic polypeptide according to any one of embodiments 1-21 wherein the protraction moiety is covalently fused to said polypeptide via a peptidic amide bond.
24. The hFGF23 antagonistic polypeptide according to embodiment 21 or 22 wherein the protraction moiety comprises at least two acidic groups wherein one acidic group is located terminally.
25. The hFGF23 antagonistic polypeptide according to embodiment 21, 22 or 24 wherein the protraction moiety comprises a C16, C17, C18, C19, C20, C21 or C22 fatty acid, such as a C16, C17, C18, C19 or C20 fatty diacid.
26. The hFGF23 antagonistic polypeptide according to embodiment 21, 22, 24 or 25 wherein said protraction moiety comprises a protractor “P” selected from the group consisting of:

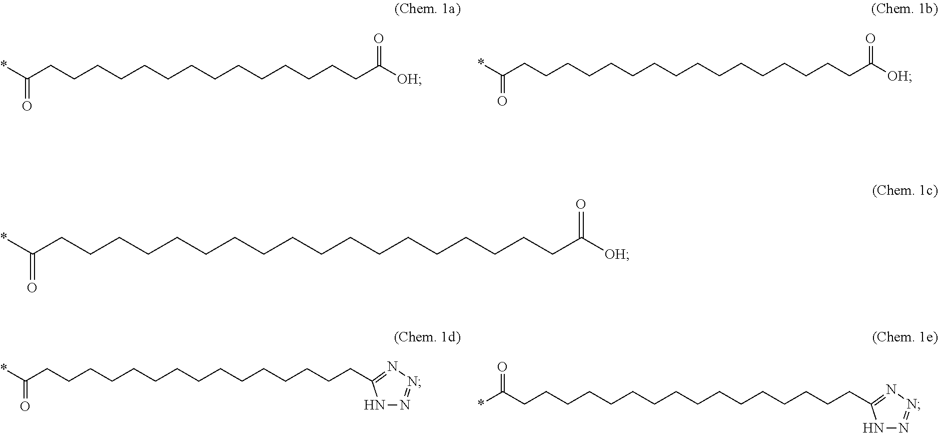 [SMILES: *C(=O)CCCCCCCCCCCCCCC(=O)O ; *C(=O)CCCCCCCCCCCCCCCCC(=O)O ; *C(=O)CCCCCCCCCCCCCCCCCCC(=O)O ; *C(=O)CCCCCCCCCCCCCCCc1nnn[nH]1 ; *C(=O)CCCCCCCCCCCCCCCCc1nnn[nH]1]

(Chem. 1f, SEQ ID NO: 9)
QRLMEDICLPRWGCLWEDDF-*;
and
(Chem. 1f, SEQ ID NO: 9)
*-QRLMEDICLPRWGCLWEDDF;

wherein * represents the attachment point to either a linker (L_{P}) or the hFGF23 antagonistic polypeptide.
27. The hFGF23 antagonistic polypeptide according to any one of embodiments 1-22 or 24-26 wherein the polypeptide is a polypeptide derivative comprising a protraction moiety wherein said protraction moiety comprises a linker “L_{P}” selected from the group consisting of:
*—NH—(CH₂)₂—(O—(CH₂))ₖ—O—(CH₂)ₙ—CO—* (chem. 2a), wherein k is an integer in the range of 1-5, and n is an integer in the range of 1-5;

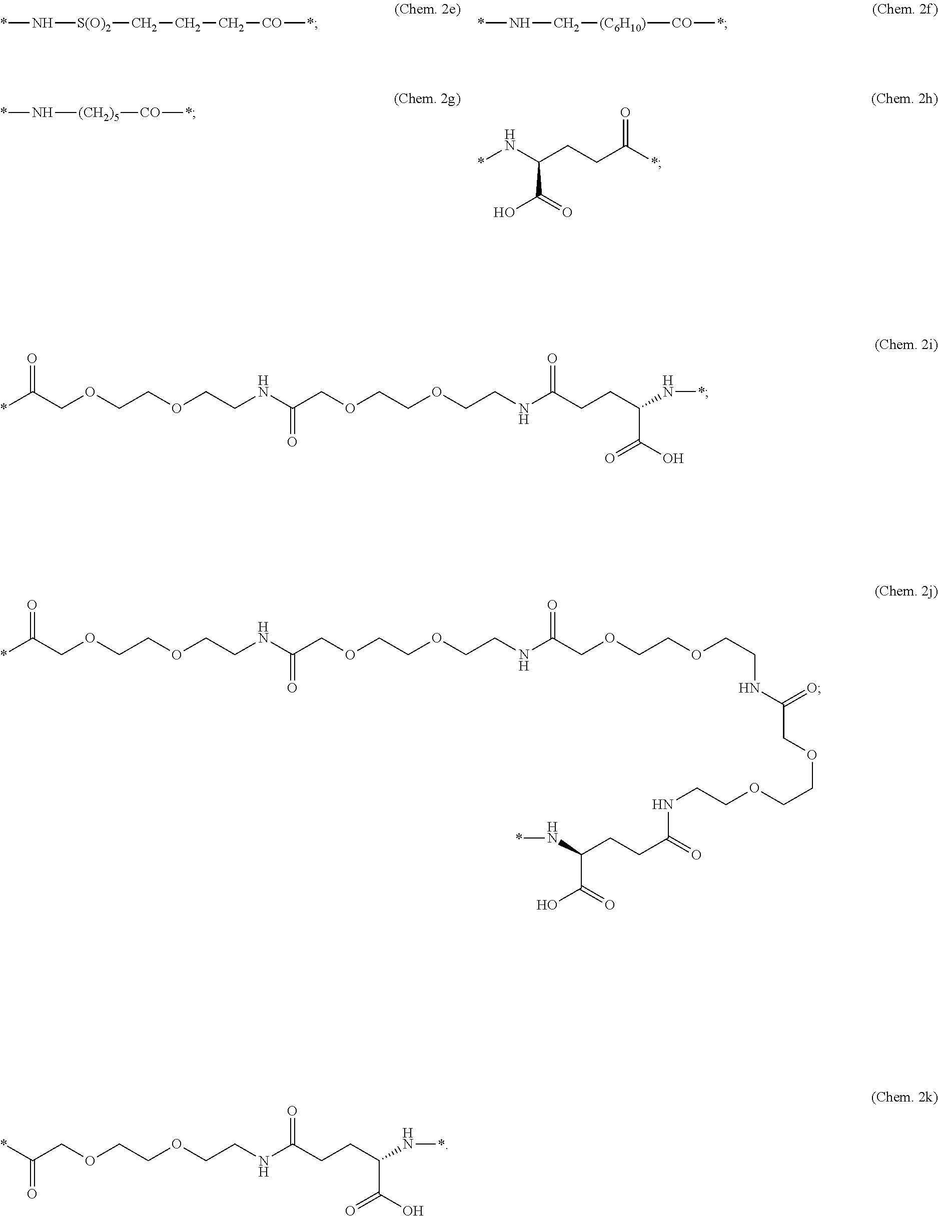

28. The hFGF23 antagonistic polypeptide according any one of embodiments 21-27 wherein the polypeptide is a polypeptide derivative comprising a protraction moiety wherein said protraction moiety is attached to the N-terminus amino acid residue of said polypeptide.
29. The hFGF23 antagonistic polypeptide according any one of embodiments 21-27 wherein the polypeptide is a polypeptide derivative comprising a protraction moiety wherein said protraction moiety is attached to the C-terminus amino acid residue of said polypeptide.
30. The hFGF23 antagonistic polypeptide according any one of embodiments 21-22 or 24-29 wherein the polypeptide is a polypeptide derivative comprising a protraction moiety wherein said protraction moiety is covalently attached to the side chain of a cysteine residue or a lysine residue in said polypeptide.
31. A compound selected from the group consisting of:

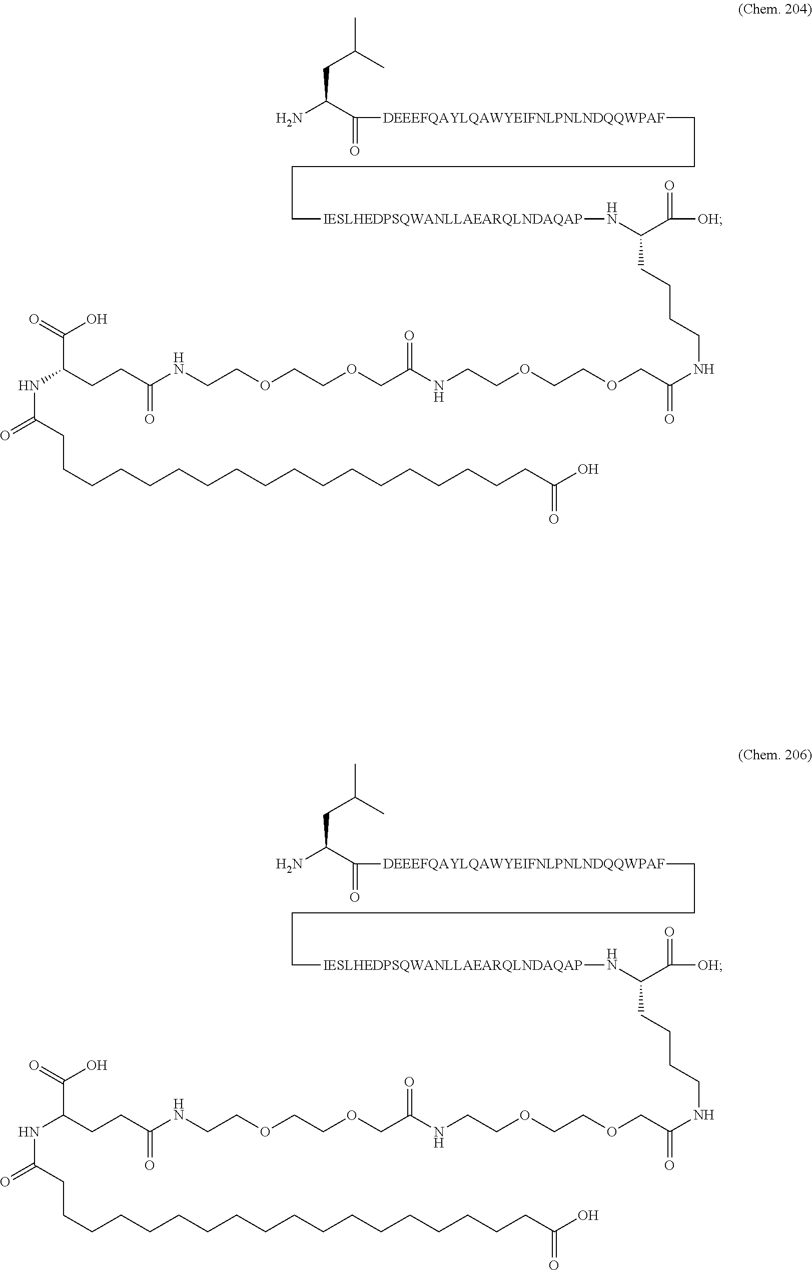

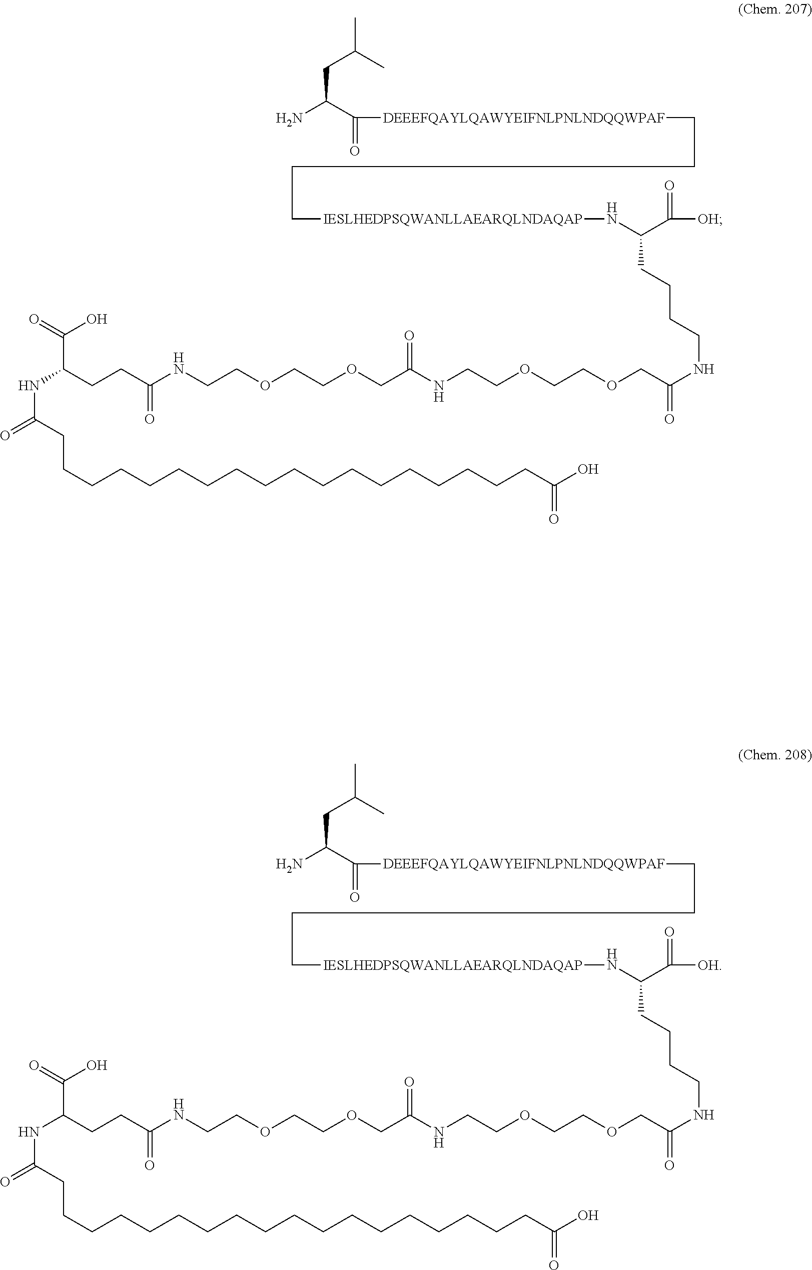

32. A hFGF23 antagonistic polypeptide derivative capable of binding hFGF23 represented by the structure:

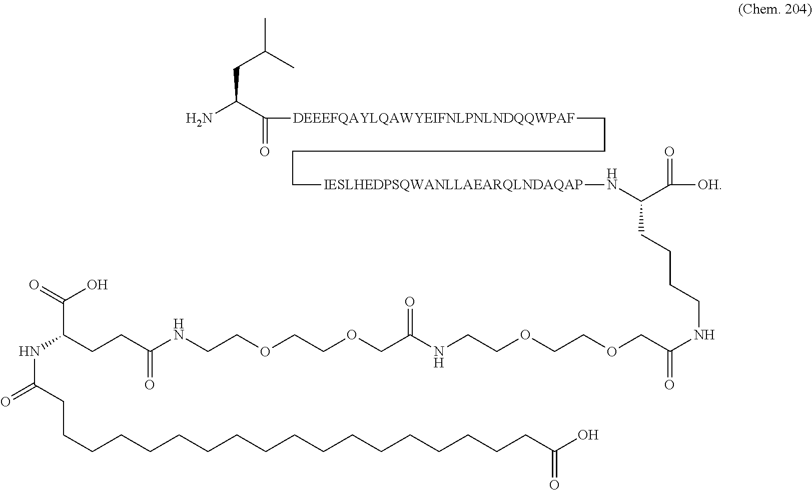

33. A hFGF23 antagonistic polypeptide derivative capable of binding to hFGF23 represented by the structure:

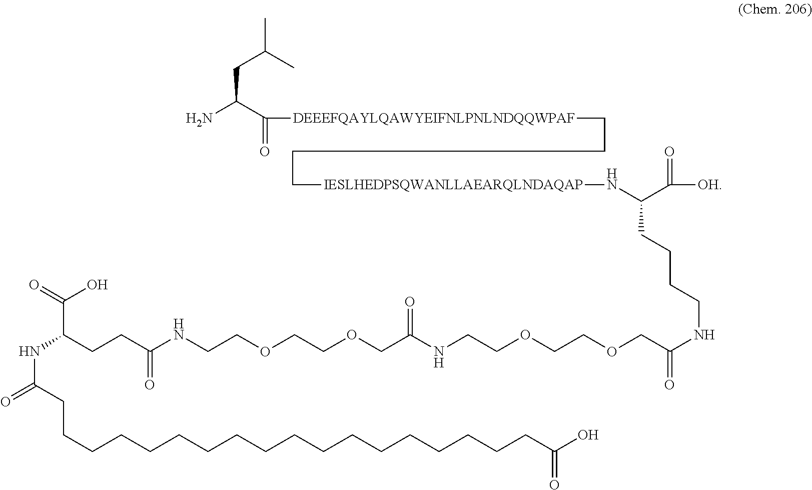

34. A hFGF23 antagonistic polypeptide derivative capable of binding to hFGF23 represented by the structure:

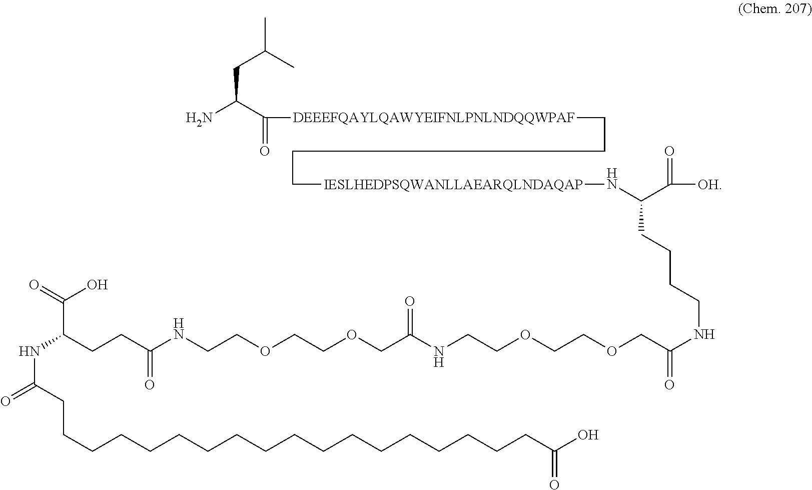

35. A hFGF23 antagonistic polypeptide derivative capable of binding to hFGF23 represented by the structure:

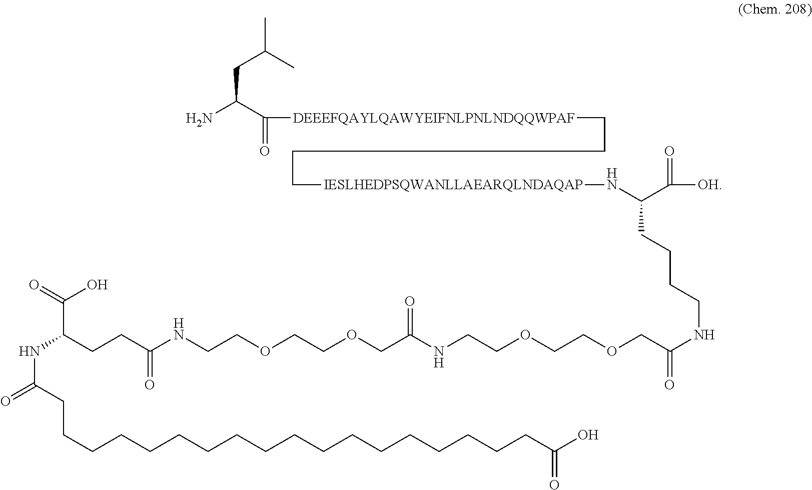

36. The compound according to embodiment 31 wherein the compound is a hFGF23 antagonistic polypeptide derivative capable of binding hFGF23.
37. The hFGF23 antagonistic polypeptide or polypeptide derivative according to any one of the preceding embodiments wherein the polypeptide or polypeptide derivative is a cross-species reactive polypeptide or polypeptide derivative.
38. The hFGF23 antagonistic polypeptide or polypeptide derivative according to the preceding embodiment wherein the polypeptide or polypeptide derivative is capable of binding to one or more of cynomolgus monkey FGF23, dog FGF23 and pig FGF23.
39. A pharmaceutical composition comprising the hFGF23 antagonistic polypeptide, polypeptide derivative or compound according to any one of the preceding embodiments and one or more pharmaceutically acceptable excipients.
40. The hFGF23 antagonistic polypeptide derivative, polypeptide derivative, compound, or composition according to any one of the preceding embodiments for use in medicine.
41. The hFGF23 antagonistic polypeptide derivative, polypeptide derivative, compound or composition according to any one of the preceding embodiments for use in the treatment of a disease characterized by elevated hFGF23 concentrations compared to that in healthy subjects.
42. The hFGF23 antagonistic polypeptide, polypeptide derivative, compound or composition according to any one of embodiments 1-39 for use in the treatment of a hypophosphatemic disorder.
43. The hFGF23 antagonistic polypeptide, polypeptide derivative, compound or composition according to any one of embodiments 1-39 for use in the treatment of tumor-induced osteomalacia, fibrous dysplasia, McCune-Albright syndrome, autosomal dominant hypophosphatemic rickets (ADHR) or X-linked hypophosphatemic rickets (XLH).
44. The hFGF23 antagonistic polypeptide, polypeptide derivative, compound or composition according to any one of embodiments 1-39 for use in the treatment of X-linked hypophosphatemic rickets (XLH).
45. A method of inhibiting hFGF23-induced signalling in patients in need thereof comprising administering to said subject the hFGF23 antagonistic polypeptide, polypeptide derivative, compound or composition according to any one of embodiments 1-39.
46. A method of treating a hypophosphatemic disease characterized by elevated hFGF23 concentrations compared to that observed in healthy subjects comprising administering to said subject the hFGF23 antagonistic polypeptide, polypeptide derivative, compound or composition according to any one of embodiments 1-39.
47. A method for increasing serum phosphate concentration in a subject suffering from a hypophosphatemic disease characterized by elevated hFGF23 concentrations compared to that observed in healthy subjects comprising administering to said subject the hFGF23 antagonistic polypeptide, polypeptide derivative, compound or composition according to any one of embodiments 1-39.
48. A method of treating a subject suffering from a hypophosphatemic disease, such as tumor-induced osteomalacia, fibrous dysplasia, McCune-Albright syndrome, autosomal dominant hypophosphatemic rickets (ADHR) or X-linked hypophosphatemic rickets (XLH), comprising administering to said subject the hFGF23 antagonistic polypeptide, polypeptide derivative, compound or composition according to any one of embodiments 1-39.
49. A method of treating a subject suffering from X-linked hypophosphatemic rickets (XLH), comprising administering to said subject the hFGF23 antagonistic polypeptide, polypeptide derivative, compound or composition according to any one of embodiments 1-39.
50. Use of the hFGF23 antagonistic polypeptide, polypeptide derivative, compound or composition according to any one of embodiments 1-39 for the manufacture of a medicament for the treatment of a subject in need thereof.
51. Use of the hFGF23 antagonistic polypeptide, polypeptide derivative, compound or composition according to any one of embodiments 1-39 for the manufacture of a medicament for the treatment of a hypophosphatemic disease, such as X-linked hypophosphatemic rickets (XLH).
52. A kit comprising the hFGF23 antagonistic polypeptide, polypeptide derivative, compound or composition according to any one of embodiments 1-39 and instructions for use.
53. A polynucleotide encoding the hFGF23 antagonistic polypeptide, polypeptide derivative or compound according to any one of embodiments 1-38.
54. An expression vector encoding a polynucleotide according to the preceding embodiment.
55. A host cell which produces the hFGF23 antagonistic polypeptide, polypeptide derivative or compound according to any one of embodiments 1-38.
56. A method of producing a hFGF23 antagonistic polypeptide or polypeptide derivative or compound according to any one of embodiments 1-38 comprising
   a) culturing a host cell according to the preceding embodiment under conditions permissive of expression of the polypeptide encoded by the expression vector of embodiment 54,
   b) isolating said polypeptide,
   c) optionally attaching a protraction moiety to said isolated polypeptide.
57. A hFGF23 antagonistic polypeptide, polypeptide derivative or compound obtainable by the method of embodiment 56.

### EXAMPLES

The below experimental section starts with a list of abbreviations and is followed by a section on the general methods for compound preparation and a section on the methods for measuring properties relevant for the exposure profile and other properties of the compounds. A number of specific examples have been included in each of the sections to illustrate the invention. All example compounds were prepared according to the general methods described herein.

### ABBREVIATIONS

Ado: 8-amino-3,6-dioxaoctanoic acid
Boc: tert-butyloxycarbonyl
Collidine: 2,4,6-trimethylpyridine
DCM: Dichloromethane
DIC: N,N′-diisopropylcarbodiimide
DMF: N,N-dimethylformamide
DTT: 1,4-dithiothreitol
FBS: fetal bovine serum
Fmoc: 9-fluorenylmethyloxycarbonyl
HEPES: 4-(2-hydroxyethyl)-1-piperazineethanesulfonic acid
HFIP: 1,1,1,3,3,3-hexafluoro-2-propanol or hexafluoroisopropanol
HPLC: High Performance Liquid Chromatography
i.v.: intravenous
LC: Liquid Chromatography
LCMS: Liquid Chromatography Mass Spectroscopy
MeCN: Acetonitrile
MQ: Milli-Q
MS: Mass Spectroscopy
MSA: Multiple Sequence Alignment
Mtt: 4-methyltrityl
NMP: N-methyl-2-pyrrolidone
OtBu: tert-butoxy
OXYMA PURE®: Ethyl cyano(hydroxyamino)acetate
OVA: Ovalbumin
Pbf: 2,2,4,6,7-pentamethyldihydrobenzofuran-5-sulfonyl
PBS: Phosphate Buffered Saline
RP: Reverse Phase
RP-HPLC: Reverse Phase High Performance Liquid Chromatography
RT: Room Temperature
RU: Response Units
s.c.: subcutaneous
SPPS: Solid Phase Peptide Synthesis
SPR: Surface Plasmon Resonance
tBu: tert-butyl
TFA: trifluoroacetic acid
TIPS: triisopropylsilane
Trt: triphenylmethyl (trityl)
UPLC: Ultra Performance Liquid Chromatography
UV: Ultraviolet

### Methods for Peptide Synthesis, Purification and Analysis

The Fmoc-protected amino acid derivatives used were: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH and Fmoc-Val-OH unless mentioned otherwise and were procured from Gyros Protein Technologies AB. Fmoc-Glu-OtBu, and resins for solid phase peptide synthesis were obtained from Novabiochem (Merck Millipore). Fmoc-Ado-OH was purchased from Bachem. Triisopropylsilane was purchased from Sigma. Fmoc-8-amino-3,6-dioxaoctanoic acid (Fmoc-Ado-OH) was purchased from Flamma Group, Italy. 16-(tert-Butoxy)-16-oxohexadecanoic acid was purchased from ABCR, 18-(tert-butoxy)-18-oxooctadecanoic acid was purchased from Solvias, and 20-(tert-butoxy)-20-oxoeicosanoic acid was prepared as is known in the art, e.g. as described in WO 2010102886 A1. Oxyma was purchased from Gyros Protein Technologies. N,N-dimethylformamide, piperidine, N,N-diisopropylcarbodiimide, trifluoroacetic acid and 1,1,1,3,3,3-hexafluoro-2-propanol were obtained from Biosolve. HPLC grade acetonitrile was procured from Merck.

Automated Fmoc SPPS was performed on a Symphony™ X (Gyros Protein Technologies AB). Fmoc-deprotection was achieved with 2×10 min treatments of 20 vol. % piperidine in 0.1 M Oxyma in DMF. Peptide couplings were performed using DIC/collidine. Amino acid/Oxyma solutions (0.3 M/0.3 M in DMF at a molar excess of 5-fold) were added to the resin followed by the same molar equivalent of DIC (1.5 M in DMF) and the same molar equivalent of collidine (1.5 M in DMF). The coupling procedure was allowed to proceed for 60 min unless specified otherwise.

After synthesis of polypeptide backbone, the protraction moiety was selectively installed on the resin-bound polypeptide via orthogonally protected amino acids such as Fmoc-Lys(Mtt)-OH in the desired position(s). The Mtt group was selectively removed by treatment with HFIP/DCM/TIPS (75:22.5:2.5) (3×15 min). Following this, the resin-bound polypeptide was subjected to treatment with 20 vol. % piperidine in 0.1 M Oxyma in DMF (1×5 min). Following deprotection, the resin was subsequently washed with DCM and DMF before the protraction moiety was introduced using standard SPPS procedures described above.

Global deprotection of resin bound peptides was performed after synthesis, and the resin was washed with DCM, and then subjected to a 2-3 h treatment with TFA/TIPS/DTT/water (95:2.5:2.5:2.5, v/v/v/v). The treatment was followed by precipitation in ice cold diethylether and collection by centrifugation. The pellet was resuspended in diethylether before being centrifuged once more. The washed pellet was dissolved in a suitable mixture of water, MeCN and acetic acid before purification by reversed-phase preparative HPLC. Crude peptides containing cysteine in position Xₕ and X₅₆, was folded before purification, by dissolving the crude peptide in DMSO (10 ml) followed by addition of 30 mM HEPES-buffer (40 ml, pH 7.8, aq) and stirred overnight. pH was lowered to 3 with trifluoroacetic acid before purification by reversed-phase preparative HPLC.

Purification of polypeptides and final analogues was done by preparative purification on a Waters™ Deltaprep 4000 Preparative Chromatography System using a reversed phase HPLC column. Elution was performed using a mobile phase composed of 0.1 vol. % TFA in MILLI-Q® water (Solvent A) and 0.1 vol. % TFA in HPLC grade acetonitrile (Solvent B) utilizing a linear gradient.

Elution was performed using a mobile phase composed of 0.1 vol. % TFA, 10 vol. % acetonitrile in Milli-Q water (Solvent A) and 0.1 vol. % TFA in HPLC grade acetonitrile (Solvent B) utilizing a linear gradient. Relevant fractions were assessed by LC-MS and purity of pooled fractions was assessed by analytical HPLC or UPLC. Fractions containing the pure target peptide were pooled and freeze dried.

### Analytical Procedures

TABLE 3
LC-MS Conditions
Method M64:

| | |
|---|---|
| System | LC-system: Waters ™ Acquity UPLC H Class |
| | Detector: Waters Xevo G2-XS Qtof |
| | Detection λ = 214 nm |
| Detector | Ionisation method: Electrospray |
| setup | Scanning range: 50-4000 m/z |
| | Operating mode: MS resolution mode (positive) |
| | Voltage: Capillary 3.00 kV, Sample cone 40 V, Source 80 V |
| | Temperature: Source 150° C., Desolvation 350° C. |
| | Scantime: 0.500 s |
| | Interscan delay: 0.014 s |
| Lockspray ™ | A solution of Leucine Enkephalin in 50 vol. % acetonitrile in Milli-Q water (0.2 |
| calibration | mg/L) was used as a lock mass for data calibration and sampled every 20 |
| | seconds. Lock mass: 556.2771 m/z |
| Eluents | Solvent A: 100 vol. % Milli-Q water |
| | Solvent B: 100 vol. % acetonitrile |
| | Solvent C: 97.9 vol. % Milli-Q water, 2 vol. % formic acid, 0.1 vol. % TFA |
| LC conditions | Column: Waters ™ Acquity BEH, C18, 1.7 μm, 2.1 mm × 50 mm |
| C18 column | Flow rate: 0.4 mL/min |
| (M64) | Gradient: 5 to 95% B over 4 mins maintaining a composition of 5% C |
| | throughout. Column oven temperature: 60° C. |

### Method: M111

LCMS111 (M111) is performed on a setup consisting of a Waters Acquity UPLC system with a Tunable UV detector (TUV) and a Waters Acquity QDa Mass Spectrometer (QDa). Eluents: A: Water with 0.05% trifluoroacetic acid; B: Acetonitrile 0.05% trifluoroacetic acid. The analysis is performed at 40° C. by injecting an appropriate volume of the sample (preferably 2-10 μl) onto the column which is eluted with a gradient of A and B. The UPLC conditions, detector settings and mass spectrometer settings are: Column: Waters Acquity UPLC BEH, C18, 1.7 μm, 2.1 mm×50 mm. Gradient: Linear 10%-90% acetonitrile during 1.6 min at 0.9 ml/min. UV detection: 214 nm using the TUV detector. MS detection: Atmospheric Pressure Ionization Electrospray (API-ES) with a scan range of 100-1250 Da using the QDa detector operating in positive mode.

### Compounds

Table 4 below lists Chem no., the SEQ ID NO, full backbone sequences, the residue R1 to which the linker attaches, the linker and the protractor. Selected—non-limiting—examples are shown below:

### Chem. 4, SEQ ID NO:53,

VDNREFNAYLQAWYEIFNLPNLNDNQWPAFIESLHEDPSQWANLLAEARRLNDAQAKE, R1: 57K, Linker: L1, Protractor: Chem 1b. The structure is shown below where the linker is attached to the epsilon-amino group of lysine in position 57.

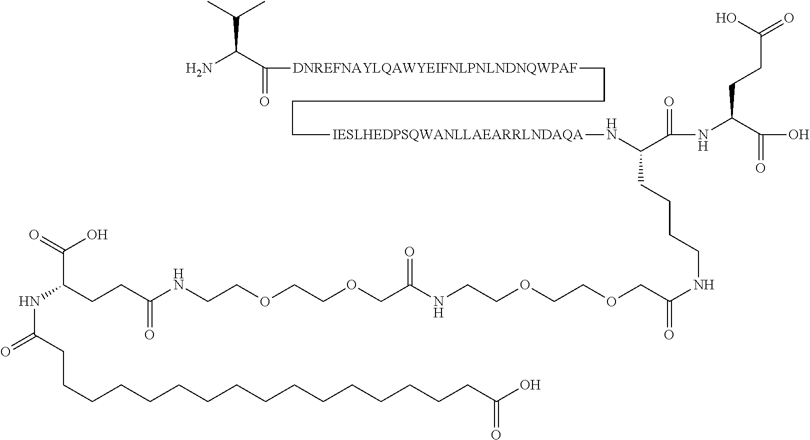

### Chem. 106, SEQ ID NO:138,

LDEEEFQAYLQAWYEIFNLPNLNDNQWPAFIESLHEDPSQWANLLAEARRLNDAQAPK, 58K, Linker: L1, Protractor: Chem 1c. The structure is shown below where the linker is attached to the epsilon-amino group of lysine in position 58.

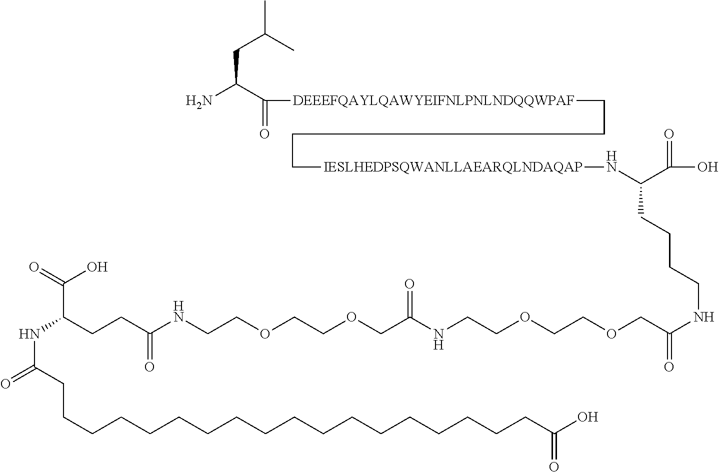

### Chem. 246, SEQ ID NO:274,

VDQEEFQAYLQAWYEIFNLPCLNDNQWPAFIESLHEDPSQWDNLLEEARRLNDAQCPK, 58K, Linker: L1, Protractor: Chem 1b. The structure is shown below where the linker is attached to the epsilon-amino group of lysine in position 58. Furthermore, when two cysteines (C) are present in the sequence in position Xₕ and X₅₆, it is understood by the skilled person, that a disulfide bridge is formed between the two cysteines, as shown in the structure below.

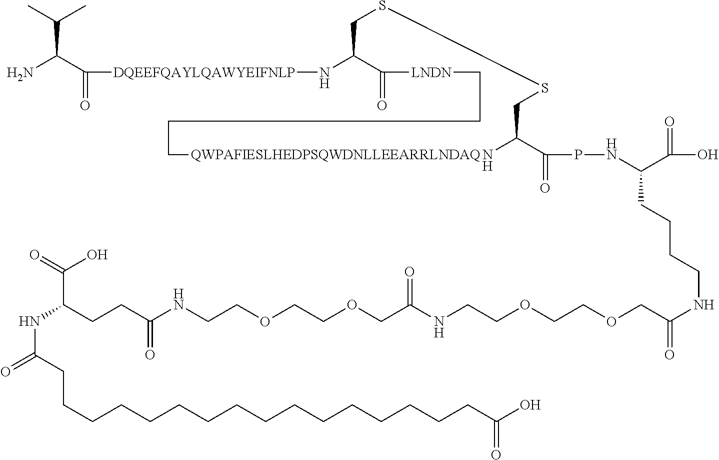

In Table 4 below, examples of compounds of the invention are disclosed. The table provides amino acid residue sequences and information on protraction moieties as well as their point of attachment to the hFGF23 antagonistic polypeptide. The Linker and Protractor columns provide a designation of the linker and protractor used, see also Table 1 and Table 2 above. Table 5 below provides technical information on the compounds, such as molecular weight and m/z values.

TABLE 4
hFGF23 antagonistic polypeptide and polypeptide derivatives

| | SEQ | | | | Protract |
|---|---|---|---|---|---|
| | ID | | | Linker | or (“P”), |
| Chem. | NO | Sequence | R1 | (“Lp”) | Chem. |
| 3 | 10 | VDNREFNAYLQAWYEIFNLPNLNDNQWPAFI | | | |
| | | ESLHEDPSQWANLLAEARRLNDAQAPE | | | |
| 4 | 53 | VDNREFNAYLQAWYEIFNLPNLNDNQWPAFI | 57K | L1 | 1b |
| | | ESLHEDPSQWANLLAEARRLNDAQAKE | | | |
| 5 | 54 | VDNTLYPLYLQAWYEIFNLPNLNDNQWPAFI | 53K | L1 | 1b |
| | | ESLHEDPSQWANLLAEARRLNKAQAPE | | | |
| 6 | 55 | VDNREFNAYLQAWYEIFNLPNLNDNQWPAFI | 53K | L1 | 1b |
| | | ESLHEDPSQWANLLAEARRLNKAQAPE | | | |
| 7 | 56 | VDNTLYPLYLQAWYEIFNLPNLNDNQWPAFI | 46K | L1 | 1b |
| | | ESLHEDPSQWANLLKEARRLNDAQAPE | | | |
| 8 | 57 | VDNREFNAYLQAWYEIFNLPNLNDNQWPAFI | 46K | L1 | 1b |
| | | ESLHEDPSQWANLLKEARRLNDAQAPE | | | |
| 9 | 58 | LDNDEFQLYLQAWYEIFNLPNLNDNQWPAFI | 58K | L1 | 1b |
| | | ESLHDDPSQWANLLEEARRLNDAQAPK | | | |
| 10 | 59 | LDNDEFTLYLQAWYEIFNLPNLNDNQWPAFI | 58K | L1 | 1b |
| | | ESLHDDPSQWANLLEEARRLNDAQAPK | | | |
| 11 | 60 | LDNTEFILYLQAWYEIFNLPNLNDNQWPAFI | 58K | L1 | 1b |
| | | ESLHDDPSQWANLLEEARRLNDAQAPK | | | |
| 12 | 61 | LDNDTFILYLQAWYEIFNLPNLNDNQWPAFI | 58K | L1 | 1b |
| | | ESLHDDPSQWANLLEEARRLNDAQAPK | | | |
| 13 | 62 | LDNDEFILYLQAWYEIFNLPNLNDNQWPAFI | 58K | L1 | 1b |
| | | ESLHADPSQWANLLEEARRLNDAQAPK | | | |
| 14 | 63 | VDNDEFILYLQAWYEIFNLPNLNDNQWPAFI | 58K | L1 | 1b |
| | | ESLHADPSQWANLLAEARRLNDAQAPK | | | |
| 15 | 64 | LDNDEFILYLQAWYEIFNLPNLNDNQWPAFI | 58K | L1 | 1b |
| | | ESLHADPSQWANLLAEARRLNDAQAPK | | | |
| 16 | 65 | IDNDEFILYLQAWYEIFNLPNLNDNQWPAFI | 58K | L1 | 1b |
| | | ESLHDDPSQWANLLAEARRLNDAQAPK | | | |
| 17 | 66 | NDNDEFILYLQAWYEIFNLPNLNDNQWPAFI | 58K | L1 | 1b |
| | | ESLHDDPSQWANLLAEARRLNDAQAPK | | | |
| 18 | 67 | VDQDEFILYLQAWYEIFNLPNLNDNQWPAFI | 58K | L1 | 1b |
| | | ESLHDDPSQWANLLAEARRLNDAQAPK | | | |
| 19 | 68 | VDNTEFILYLQAWYEIFNLPNLNDNQWPAFI | 58K | L1 | 1b |
| | | ESLHDDPSQWANLLAEARRLNDAQAPK | | | |
| 20 | 69 | VDNNEFILYLQAWYEIFNLPNLNDNQWPAFI | 58K | L1 | 1b |
| | | ESLHDDPSQWANLLAEARRLNDAQAPK | | | |
| 21 | 70 | VDNHEFILYLQAWYEIFNLPNLNDNQWPAFI | 58K | L1 | 1b |
| | | ESLHDDPSQWANLLAEARRLNDAQAPK | | | |
| 22 | 71 | VDNREFILYLQAWYEIFNLPNLNDNQWPAFI | 58K | L1 | 1b |
| | | ESLHDDPSQWANLLAEARRLNDAQAPK | | | |
| 23 | 72 | VDNDTFILYLQAWYEIFNLPNLNDNQWPAFI | 58K | L1 | 1b |
| | | ESLHDDPSQWANLLAEARRLNDAQAPK | | | |
| 24 | 73 | VDNDVFILYLQAWYEIFNLPNLNDNQWPAFI | 58K | L1 | 1b |
| | | ESLHDDPSQWANLLAEARRLNDAQAPK | | | |
| 25 | 74 | VDNDEFQLYLQAWYEIFNLPNLNDNQWPAFI | 58K | L1 | 1b |
| | | ESLHDDPSQWANLLAEARRLNDAQAPK | | | |
| 26 | 75 | VDNDEFNLYLQAWYEIFNLPNLNDNQWPAFI | 58K | L1 | 1b |
| | | ESLHDDPSQWANLLAEARRLNDAQAPK | | | |
| 27 | 76 | VDNDEFLLYLQAWYEIFNLPNLNDNQWPAFI | 58K | L1 | 1b |
| | | ESLHDDPSQWANLLAEARRLNDAQAPK | | | |
| 28 | 77 | VDNDEFALYLQAWYEIFNLPNLNDNQWPAFI | 58K | L1 | 1b |
| | | ESLHDDPSQWANLLAEARRLNDAQAPK | | | |
| 29 | 78 | VDNDEFRLYLQAWYEIFNLPNLNDNQWPAFI | 58K | L1 | 1b |
| | | ESLHDDPSQWANLLAEARRLNDAQAPK | | | |
| 30 | 79 | VDNDEFGLYLQAWYEIFNLPNLNDNQWPAFI | 58K | L1 | 1b |
| | | ESLHDDPSQWANLLAEARRLNDAQAPK | | | |
| 31 | 80 | VDNDEFPLYLQAWYEIFNLPNLNDNQWPAFI | 58K | L1 | 1b |
| | | ESLHDDPSQWANLLAEARRLNDAQAPK | | | |
| 32 | 81 | VDNDEFHLYLQAWYEIFNLPNLNDNQWPAFI | 58K | L1 | 1b |
| | | ESLHDDPSQWANLLAEARRLNDAQAPK | | | |
| 33 | 82 | VDNDEFVLYLQAWYEIFNLPNLNDNQWPAFI | 58K | L1 | 1b |
| | | ESLHDDPSQWANLLAEARRLNDAQAPK | | | |
| 34 | 83 | LDNDEFILYLQAWYEIFNLPWLNDNQWPAFI | 58K | L1 | 1b |
| | | ESLHADPSQWANLLEEARRLNDAQAPK | | | |
| 35 | 84 | LDNDEFILYLQAWYEIFNLPYLNDNQWPAFIE | 58K | L1 | 1b |
| | | SLHADPSQWANLLEEARRLNDAQAPK | | | |
| 36 | 85 | LDNDEFIEYLQAWYEIFNLPNLNDNQWPAFI | 58K | L1 | 1b |
| | | ESLHDDPSQRANLLAEARRLNDAQAPK | | | |
| 37 | 86 | LDNDEFILYLQAWYEIFQLPNLNDNQWPAFI | 58K | L1 | 1b |
| | | ESLHDDPSQWAQLLEEARRLNDAQAPK | | | |
| 38 | 87 | LDNDEFILYLQAWYEIFNLPWLNDNQWPAFI | 58K | L1 | 1b |
| | | ESLHDDPSQWAQLLEEARRLNDAQAPK | | | |
| 39 | 88 | LDNDEFILYLQAWYEIFNLPWLNDNQWPAFI | 58K | L1 | 1b |
| | | ESLHADPSQWAQLLEEARRLNDAQAPK | | | |
| 40 | 89 | VEGDEFNAYLQAWYEIFNLPNLNDNQWPAF | 58K | L1 | 1b |
| | | IESLHEDPSQWANLLAEARRLNDAQAPK | | | |
| 41 | 90 | VEGEEFNAYLQAWYEIFNLPNLNDNQWPAFI | 58K | L1 | 1b |
| | | ESLHEDPSQWANLLAEARRLNDAQAPK | | | |
| 42 | 91 | VDNTIFNAYLQAWYEIFNLPNLNDNQWPAFI | 58K | L1 | 1b |
| | | ESLHEDPSQWANLLAEARRLNDAQAPK | | | |
| 43 | 92 | VDNTVFNAYLQAWYEIFNLPNLNDNQWPAFI | 58K | L1 | 1b |
| | | ESLHEDPSQWANLLAEARRLNDAQAPK | | | |
| 44 | 93 | VDNDLFNAYLQAWYEIFNLPNLNDNQWPAFI | 58K | L1 | 1b |
| | | ESLHEDPSQWANLLAEARRLNDAQAPK | | | |
| 45 | 94 | VDNDIFNAYLQAWYEIFNLPNLNDNQWPAFI | 58K | L1 | 1b |
| | | ESLHEDPSQWANLLAEARRLNDAQAPK | | | |
| 46 | 95 | VDNDVFNAYLQAWYEIFNLPNLNDNQWPAFI | 58K | L1 | 1b |
| | | ESLHEDPSQWANLLAEARRLNDAQAPK | | | |
| 47 | 96 | VENTLFNAYLQAWYEIFNLPNLNDNQWPAFI | 58K | L1 | 1b |
| | | ESLHEDPSQWANLLAEARRLNDAQAPK | | | |
| 48 | 97 | VENTIFNAYLQAWYEIFNLPNLNDNQWPAFI | 58K | L1 | 1b |
| | | ESLHEDPSQWANLLAEARRLNDAQAPK | | | |
| 49 | 98 | VENTVFNAYLQAWYEIFNLPNLNDNQWPAFI | 58K | L1 | 1b |
| | | ESLHEDPSQWANLLAEARRLNDAQAPK | | | |
| 50 | 99 | VDNTLFNAYLQAWYEIFNLPNLNDNQWPAFI | 58K | L1 | 1b |
| | | ESLHEDPSQWANLLAEARRLNDAQAPK | | | |
| 51 | 100 | VDNTLFNAYLQAWWEIFNLPNLNDNQWPAF | 58K | L1 | 1b |
| | | IESLHEDPSQWANLLAEARRLNDAQAPK | | | |
| 52 | 101 | VDNTLFNAYLQAWFEIFNLPNLNDNQWPAFI | 58K | L1 | 1b |
| | | ESLHEDPSQWANLLAEARRLNDAQAPK | | | |
| 53 | 102 | VDNTLFNAYLQAWHEIFNLPNLNDNQWPAFI | 58K | L1 | 1b |
| | | ESLHEDPSQWANLLAEARRLNDAQAPK | | | |
| 54 | 103 | VDNTEFILYLQAWWEIFNLPNLNDNQWPAFI | 58K | L1 | 1b |
| | | ESLHDDPSQWANLLAEARRLNDAQAPK | | | |
| 55 | 104 | VDNTEFILYLQAWFEIFNLPNLNDNQWPAFI | 58K | L1 | 1b |
| | | ESLHDDPSQWANLLAEARRLNDAQAPK | | | |
| 56 | 105 | VDNTEFILYLQAWHEIFNLPNLNDNQWPAFI | 58K | L1 | 1b |
| | | ESLHDDPSQWANLLAEARRLNDAQAPK | | | |
| 57 | 106 | VDNTLFILYLQAWWEIFNLPNLNDNQWPAFI | 58K | L1 | 1b |
| | | ESLHDDPSQWANLLAEARRLNDAQAPK | | | |
| 58 | 107 | VEGDEFNAYLQAWYEIFNLPNLNDNQWPAFI | 58K | L1 | 1b |
| | | ESLHEDPSQWANLLEEARRLNDAQAPK | | | |
| 59 | 108 | VEGEEFNAYLQAWYEIFNLPNLNDNQWPAFI | 58K | L1 | 1b |
| | | ESLHEDPSQWANLLEEARRLNDAQAPK | | | |
| 60 | 109 | VDNTLFNAYLQAWYEIFNLPNLNDNQWPAFI | 58K | L1 | 1b |
| | | ESLHEDPSQWANLLEEARRLNDAQAPK | | | |
| 61 | 110 | VDNTIFNAYLQAWYEIFNLPNLNDNQWPAFI | 58K | L1 | 1b |
| | | ESLHEDPSQWANLLEEARRLNDAQAPK | | | |
| 62 | 111 | VDNTVFNAYLQAWYEIFNLPNLNDNQWPAFI | 58K | L1 | 1b |
| | | ESLHEDPSQWANLLEEARRLNDAQAPK | | | |
| 63 | 112 | VDNDLFNAYLQAWYEIFNLPNLNDNQWPAFI | 58K | L1 | 1b |
| | | ESLHEDPSQWANLLEEARRLNDAQAPK | | | |
| 64 | 113 | VDNDIFNAYLQAWYEIFNLPNLNDNQWPAFI | 58K | L1 | 1b |
| | | ESLHEDPSQWANLLEEARRLNDAQAPK | | | |
| 65 | 114 | VDNDVFNAYLQAWYEIFNLPNLNDNQWPAFI | 58K | L1 | 1b |
| | | ESLHEDPSQWANLLEEARRLNDAQAPK | | | |
| 66 | 115 | VENTLFNAYLQAWYEIFNLPNLNDNQWPAFI | 58K | L1 | 1b |
| | | ESLHEDPSQWANLLEEARRLNDAQAPK | | | |
| 67 | 116 | VENTIFNAYLQAWYEIFNLPNLNDNQWPAFI | 58K | L1 | 1b |
| | | ESLHEDPSQWANLLEEARRLNDAQAPK | | | |
| 68 | 117 | VDNTLFNAYLQAWWEIFNLPNLNDNQWPAF | 58K | L1 | 1b |
| | | IESLHEDPSQWANLLEEARRLNDAQAPK | | | |
| 69 | 118 | VDNTLFNAYLQAWHEIFNLPNLNDNQWPAFI | 58K | L1 | 1b |
| | | ESLHEDPSQWANLLEEARRLNDAQAPK | | | |
| 70 | 119 | VDNTEFILYLQAWWEIFNLPNLNDNQWPAFI | 58K | L1 | 1b |
| | | ESLHDDPSQWANLLEEARRLNDAQAPK | | | |
| 71 | 120 | LDNEEFNAYLQAWYEIFNLPNLNDNQWPAFI | 58K | L1 | 1b |
| | | ESLHEDPSQWANLLAEARRLNDAQAPK | | | |
| 72 | 121 | LDNREFQAYLQAWYEIFNLPNLNDNQWPAFI | 58K | L1 | 1b |
| | | ESLHEDPSQWANLLAEARRLNDAQAPK | | | |
| 73 | 122 | VDQEEFNAYLQAWYEIFNLPNLNDNQWPAF | 58K | L1 | 1b |
| | | IESLHEDPSQWANLLAEARRLNDAQAPK | | | |
| 74 | 123 | VDEEEFNAYLQAWYEIFNLPNLNDNQWPAFI | 58K | L1 | 1b |
| | | ESLHEDPSQWANLLAEARRLNDAQAPK | | | |
| 75 | 124 | VDNEEFQAYLQAWYEIFNLPNLNDNQWPAFI | 58K | L1 | 1b |
| | | ESLHEDPSQWANLLAEARRLNDAQAPK | | | |
| 76 | 125 | LDNELFNAYLQAWYEIFNLPNLNDNQWPAFI | 58K | L1 | 1b |
| | | ESLHEDPSQWANLLAEARRLNDAQAPK | | | |
| 77 | 126 | LDQTEFNAYLQAWYEIFNLPNLNDNQWPAFI | 58K | L1 | 1b |
| | | ESLHEDPSQWANLLAEARRLNDAQAPK | | | |
| 78 | 127 | LDETEFNAYLQAWYEIFNLPNLNDNQWPAFI | 58K | L1 | 1b |
| | | ESLHEDPSQWANLLAEARRLNDAQAPK | | | |
| 79 | 128 | LDDEEFNAYLQAWYEIFNLPNLNDNQWPAFI | 58K | L1 | 1b |
| | | ESLHEDPSQWANLLAEARRLNDAQAPK | | | |
| 80 | 129 | LDNEEFQAYLQAWYEIFNLPNLNDNQWPAFI | 58K | L1 | 1b |
| | | ESLHEDPSQWANLLAEARRLNDAQAPK | | | |
| 81 | 130 | VDETLFNAYLQAWYEIFNLPNLNDNQWPAFI | 58K | L1 | 1b |
| | | ESLHEDPSQWANLLAEARRLNDAQAPK | | | |
| 82 | 131 | VDQTEFQAYLQAWYEIFNLPNLNDNQWPAFI | 58K | L1 | 1b |
| | | ESLHEDPSQWANLLAEARRLNDAQAPK | | | |
| 83 | 132 | VDQEEFQAYLQAWYEIFNLPNLNDNQWPAFI | 58K | L1 | 1b |
| | | ESLHEDPSQWANLLAEARRLNDAQAPK | | | |
| 84 | 133 | VDEEEFQAYLQAWYEIFNLPNLNDNQWPAFI | 58K | L1 | 1b |
| | | ESLHEDPSQWANLLAEARRLNDAQAPK | | | |
| 85 | 134 | VDNTLFQAYLQAWYEIFNLPNLNDNQWPAFI | 58K | L1 | 1b |
| | | ESLHEDPSQWANLLAEARRLNDAQAPK | | | |
| 86 | 135 | LDNELFQAYLQAWYEIFNLPNLNDNQWPAFI | 58K | L1 | 1b |
| | | ESLHEDPSQWANLLAEARRLNDAQAPK | | | |
| 87 | 136 | LDQTLFNAYLQAWYEIFNLPNLNDNQWPAFI | 58K | L1 | 1b |
| | | ESLHEDPSQWANLLAEARRLNDAQAPK | | | |
| 88 | 137 | LDQEEFQAYLQAWYEIFNLPNLNDNQWPAFI | 58K | L1 | 1b |
| | | ESLHEDPSQWANLLAEARRLNDAQAPK | | | |
| 89 | 138 | LDEEEFQAYLQAWYEIFNLPNLNDNQWPAFI | 58K | L1 | 1b |
| | | ESLHEDPSQWANLLAEARRLNDAQAPK | | | |
| 90 | 139 | LDQELFNAYLQAWYEIFNLPNLNDNQWPAFI | 58K | L1 | 1b |
| | | ESLHEDPSQWANLLAEARRLNDAQAPK | | | |
| 91 | 140 | VDQTLFQAYLQAWYEIFNLPNLNDNQWPAFI | 58K | L1 | 1b |
| | | ESLHEDPSQWANLLAEARRLNDAQAPK | | | |
| 92 | 141 | LDQELFQAYLQAWYEIFNLPNLNDNQWPAFI | 58K | L1 | 1b |
| | | ESLHEDPSQWANLLAEARRLNDAQAPK | | | |
| 93 | 142 | LDEELFQAYLQAWYEIFNLPNLNDNQWPAFI | 58K | L1 | 1b |
| | | ESLHEDPSQWANLLAEARRLNDAQAPK | | | |
| 94 | 143 | LDQTLFQAYLQAWYEIFNLPNLNDNQWPAFI | 58K | L1 | 1b |
| | | ESLHEDPSQWANLLAEARRLNDAQAPK | | | |
| 95 | 144 | LDDTLFQAYLQAWYEIFNLPNLNDNQWPAFI | 58K | L1 | 1b |
| | | ESLHEDPSQWANLLAEARRLNDAQAPK | | | |
| 96 | 145 | VDNDEFNAYLQAWYEIFNLPNLNDNQWPAFI | 58K | L1 | 1b |
| | | ESLHEDPSQWANLLAEARRLNDAQAPK | | | |
| 97 | 146 | VDNREFIAYLQAWYEIFNLPNLNDNQWPAFI | 58K | L1 | 1b |
| | | ESLHEDPSQWANLLAEARRLNDAQAPK | | | |
| 98 | 147 | VDNREFLAYLQAWYEIFNLPNLNDNQWPAFI | 58K | L1 | 1b |
| | | ESLHEDPSQWANLLAEARRLNDAQAPK | | | |
| 99 | 148 | VDNREFNLYLQAWYEIFNLPNLNDNQWPAFI | 58K | L1 | 1b |
| | | ESLHEDPSQWANLLAEARRLNDAQAPK | | | |
| 100 | 149 | LDNEEFQAYLQAWYEIFNLPNLNDNQWPAFI | 58K | L1 | 1b |
| | | ESLHEDPSQWDNLLEEARRLNDAQAPK | | | |
| 101 | 150 | VDQEEFQAYLQAWYEIFNLPNLNDNQWPAF | 58K | L1 | 1b |
| | | IESLHEDPSQWDNLLAEARRLNDAQAPK | | | |
| 102 | 151 | VDNREFVAYLQAWYEIFNLPNLNDNQWPAFI | 58K | L1 | 1b |
| | | ESLHEDPSQWANLLAEARRLNDAQAPK | | | |
| 103 | 152 | VDNREFNAYLAAWYEIFNLPNLNDNQWPAFI | 58K | L1 | 1b |
| | | ESLHEDPSQWANLLAEARRLNDAQAPK | | | |
| 104 | 153 | VDNTEFQAYLQAWYEIFNLPNLNDNQWPAFI | 58K | L1 | 1b |
| | | ESLHEDPSQWANLLAEARRLNDAQAPK | | | |
| 105 | 154 | GEGTEFQAYLQAWYEIFNLPNLNDNQWPAFI | 58K | L1 | 1b |
| | | ESLHEDPSQWANLLAEARRLNDAQAPK | | | |
| 106 | 138 | LDEEEFQAYLQAWYEIFNLPNLNDNQWPAFI | 58K | L1 | 1c |
| | | ESLHEDPSQWANLLAEARRLNDAQAPK | | | |
| 107 | 155 | LDEEEFQAYLQAWYEIFNLPNLNDNQWPAFI | 58K | L1 | 1b |
| | | ESLHEDPSQWANLLEEARRLNDAQAPK | | | |
| 108 | 156 | LENEEFQAYLQAWYEIFNLPNLNDNQWPAFI | 58K | L1 | 1b |
| | | ESLHEDPSQWANLLAEARRLNDAQAPK | | | |
| 109 | 157 | LENEEFQAYLQAWYEIFNLPNLNDNQWPAFI | 58K | L1 | 1b |
| | | ESLHEDPSQWANLLEEARRLNDAQAPK | | | |
| 110 | 158 | LENEEFQLYLQAWYEIFNLPNLNDNQWPAFI | 58K | L1 | 1b |
| | | ESLHEDPSQWANLLEEARRLNDAQAPK | | | |
| 111 | 159 | LDEEEFQAYLQAWYEIFNLPNLNDNQWPAFI | 58K | L1 | 1b |
| | | ESLHEDPSQWANLLEEARQLNDAQAPK | | | |
| 112 | 160 | LDEEEFQAYLQAWYEIFNLPNLNDNQWPAFI | 58K | L1 | 1b |
| | | ESLHEDPSQWTNLLEEARQLNDAQAPK | | | |
| 113 | 161 | LDEEEFQAYLQAWYEIFNLPNLNDNQWPAFI | 58K | L1 | 1b |
| | | ESLHEDPSQWTSLLEEARQLNDAQAPK | | | |
| 114 | 162 | LDEEEFQAYLQAWYEIFNLPHLNDNQWPAFI | 58K | L1 | 1b |
| | | ESLHEDPSQWANLLEEARRLNDAQAPK | | | |
| 115 | 163 | LDEEEFQAYLQAWYEIFNLPNLNDNQWPAFI | 58K | L1 | 1b |
| | | EALHEDPSQWANLLEEARRLNDAQAPK | | | |
| 116 | 164 | LDEEEFQAYLQAWYEIFNLPNLNDNQWPAFI | 58K | L1 | 1b |
| | | ESLHEDPSQAANLLEEARRLNDAQAPK | | | |
| 117 | 165 | LDEEEFQAYLQAWYEIFNLPNLNDNQWPAFI | 58K | L1 | 1b |
| | | EQLHEDPSQWANLLEEARRLNDAQAPK | | | |
| 118 | 166 | LDEEEFQAYLQAWYEIFQLPNLNDNQWPAFI | 58K | L1 | 1b |
| | | ESLHEDPSQWANLLAEARRLNDAQAPK | | | |
| 119 | 167 | LDEEEFQAYLQAWYEIFNLPNLQDNQWPAFI | 58K | L1 | 1b |
| | | ESLHEDPSQWANLLAEARRLNDAQAPK | | | |
| 120 | 168 | LDEEEFQAYLQAWYEIFNLPNLNDQQWPAFI | 58K | L1 | 1b |
| | | ESLHEDPSQWANLLAEARRLNDAQAPK | | | |
| 121 | 169 | LDEEEFQAYLQAWYEIFNLPNLNDNQWPAFI | 58K | L1 | 1b |
| | | ESLHEDPSQWAQLLAEARRLNDAQAPK | | | |
| 122 | 170 | LDEEEFQAYLQAWYEIFNLPNLNDDQWPAFI | 58K | L1 | 1b |
| | | ESLHEDPSQWANLLEEARRLNDAQAPK | | | |
| 123 | 171 | LDEEEFQAYLQAWYEIFNLPNLNDEQWPAFI | 58K | L1 | 1b |
| | | ESLHEDPSQWANLLEEARRLNDAQAPK | | | |
| 124 | 172 | LDEEEFQAYLQAWYEIFNLPNLNDQQWPAFI | 58K | L1 | 1b |
| | | ESLHEDPSQWANLLEEARRLNDAQAPK | | | |
| 125 | 173 | LDEEEFQAYLQAWYEIFNLPNLNDSQWPAFI | 58K | L1 | 1b |
| | | ESLHEDPSQWANLLEEARRLNDAQAPK | | | |
| 126 | 174 | LDEEEFQAYLQAWYEIFNLPNLNDTQWPAFI | 58K | L1 | 1b |
| | | ESLHEDPSQWANLLEEARRLNDAQAPK | | | |
| 127 | 175 | LDEEEFQAYLQAWYEIFNLPNLNDHQWPAFI | 58K | L1 | 1b |
| | | ESLHEDPSQWANLLEEARRLNDAQAPK | | | |
| 128 | 176 | LDEEEFQAYLQAWYEIFNLPNLNDAQWPAFI | 58K | L1 | 1b |
| | | ESLHEDPSQWANLLEEARRLNDAQAPK | | | |
| 129 | 177 | LDEEEFQAYLQAWYEIFNLPNLNDLQWPAFI | 58K | L1 | 1b |
| | | ESLHEDPSQWANLLEEARRLNDAQAPK | | | |
| 130 | 178 | LDEEEFQAYLQAWYEIFNLPNLNDNQWPAFI | 58K | L1 | 1b |
| | | ESLHEDPSQWAELLEEARRLNDAQAPK | | | |
| 131 | 179 | LDEEEFQAYLQAWYEIFNLPNLNDNQWPAFI | 58K | L1 | 1b |
| | | ESLHEDPSQWAQLLEEARRLNDAQAPK | | | |
| 132 | 180 | LDEEEFQAYLQAWYEIFNLPNLNDEQWPAFI | 58K | L1 | 1b |
| | | ESLHEDPSQWAELLEEARRLNDAQAPK | | | |
| 133 | 181 | LDEEEFQAYLQAWYEIFNLPNLNDEQWPAFI | 58K | L1 | 1b |
| | | ESLHEDPSQWAQLLEEARRLNDAQAPK | | | |
| 134 | 182 | LDEEEFQAYLQAWYEIFNLPNLNDQQWPAFI | 58K | L1 | 1b |
| | | ESLHEDPSQWAELLEEARRLNDAQAPK | | | |
| 135 | 183 | LDEEEFQAYLQAWYEIFNLPNLNDQQWPAFI | 58K | L1 | 1b |
| | | ESLHEDPSQWAQLLEEARRLNDAQAPK | | | |
| 136 | 184 | LDEEEFQAYLQAWYEIFNLPYLNDNQWPAFI | 58K | L1 | 1b |
| | | ESLHEDPSQWANLLEEARRLNDAQAPK | | | |
| 137 | 185 | LDEEEFQAYLQAWYEIFNLPNLTDNQWPAFI | 58K | L1 | 1b |
| | | ESLHEDPSQWAELLEEARRLNDAQAPK | | | |
| 138 | 186 | LDEEEFQAYLQAWYEIFNLPNLTDNQWPAFI | 58K | L1 | 1b |
| | | ESLHEDPSQWAQLLEEARRLNDAQAPK | | | |
| 139 | 187 | LDEEEFQAYLQAWYEIFNLPNLTDEQWPAFI | 58K | L1 | 1b |
| | | ESLHEDPSQWANLLEEARRLNDAQAPK | | | |
| 140 | 188 | LDEEEFQAYLQAWYEIFNLPNLTDEQWPAFI | 58K | L1 | 1b |
| | | ESLHEDPSQWAELLEEARRLNDAQAPK | | | |
| 141 | 189 | LDEEEFQAYLQAWYEIFNLPNLTDEQWPAFI | 58K | L1 | 1b |
| | | ESLHEDPSQWAQLLEEARRLNDAQAPK | | | |
| 142 | 190 | LDEEEFQAYLQAWYEIFNLPNLTDQQWPAFI | 58K | L1 | 1b |
| | | ESLHEDPSQWANLLEEARRLNDAQAPK | | | |
| 143 | 191 | LDEEEFQAYLQAWYEIFNLPNLTDQQWPAFI | 58K | L1 | 1b |
| | | ESLHEDPSQWAELLEEARRLNDAQAPK | | | |
| 144 | 192 | LDEEEFQAYLQAWYEIFNLPNLTDQQWPAFI | 58K | L1 | 1b |
| | | ESLHEDPSQWAQLLEEARRLNDAQAPK | | | |
| 145 | 193 | LDEEEFQAYLQAWYEIFNLPNLNDNQWPAFI | 58K | L1 | 1b |
| | | ESLHEEPSQWANLLEEARRLNDAQAPK | | | |
| 146 | 194 | LDEEEFQAYLQAWYEIFNLPNLNDNQWPAFI | 58K | L1 | 1b |
| | | ESLHEEPSQWAELLEEARRLNDAQAPK | | | |
| 147 | 195 | LDEEEFQAYLQAWYEIFNLPNLNDNQWPAFI | 58K | L1 | 1b |
| | | ESLHEEPSQWAQLLEEARRLNDAQAPK | | | |
| 148 | 196 | LDEEEFQAYLQAWYEIFNLPNLNDEQWPAFI | 58K | L1 | 1b |
| | | ESLHEEPSQWANLLEEARRLNDAQAPK | | | |
| 149 | 197 | LDEEEFQAYLQAWYEIFNLPNLNDEQWPAFI | 58K | L1 | 1b |
| | | ESLHEEPSQWAELLEEARRLNDAQAPK | | | |
| 150 | 198 | LDEEEFQAYLQAWYEIFNLPNLNDEQWPAFI | 58K | L1 | 1b |
| | | ESLHEEPSQWAQLLEEARRLNDAQAPK | | | |
| 151 | 199 | LDEEEFQAYLQAWYEIFNLPNLNDQQWPAFI | 58K | L1 | 1b |
| | | ESLHEEPSQWANLLEEARRLNDAQAPK | | | |
| 152 | 200 | LDEEEFQAYLQAWYEIFNLPNLNDQQWPAFI | 58K | L1 | 1b |
| | | ESLHEEPSQWAELLEEARRLNDAQAPK | | | |
| 153 | 201 | LDEEEFQAYLQAWYEIFNLPNLNDDQWPAFI | 58K | L1 | 1b |
| | | ESLHEDPSQWAQLLEEARRLNDAQAPK | | | |
| 154 | 202 | LDEEEFQAYLQAWYEIFNLPNLSDNQWPAFI | 58K | L1 | 1b |
| | | ESLHEDPSQWANLLEEARRLNDAQAPK | | | |
| 155 | 203 | LDEEEFQAYLQAWYEIFNLPNLNDSQWPAFI | 58K | L1 | 1b |
| | | ESLHEDPSQWAQLLEEARRLNDAQAPK | | | |
| 156 | 204 | LDEEEFQAYLQAWYEIFNLPNLNDTQWPAFI | 58K | L1 | 1b |
| | | ESLHEDPSQWAQLLEEARRLNDAQAPK | | | |
| 157 | 205 | LDEEEFQAYLQAWYEIFNLPNLNDAQWPAFI | 58K | L1 | 1b |
| | | ESLHEDPSQWAQLLEEARRLNDAQAPK | | | |
| 158 | 206 | LDEEEFQAYLQAWYEIFNLPNLNDLQWPAFI | 58K | L1 | 1b |
| | | ESLHEDPSQWAQLLEEARRLNDAQAPK | | | |
| 159 | 207 | LDEEEFQAYLQAWYEIFNLPNLNDGQWPAFI | 58K | L1 | 1b |
| | | ESLHEDPSQWAQLLEEARRLNDAQAPK | | | |
| 160 | 208 | LDEEEFQAYLQAWYEIFNLPNLNDDQWPAFI | 58K | L1 | 1b |
| | | ESLHEDPSQWAELLEEARRLNDAQAPK | | | |
| 161 | 209 | LDEEEFQAYLQAWYEIFQLPNLNDNQWPAFI | 58K | L1 | 1b |
| | | ESLHEDPSQWANLLEEARRLNDAQAPK | | | |
| 162 | 210 | LDEEEFQAYLQAWYEIFNLPNLNDNQWPAFI | 58K | L1 | 1b |
| | | ESLHEDPSQWASLLEEARRLNDAQAPK | | | |
| 163 | 211 | LDEEEFQAYLQAWYEIFNLPNLNDSQWPAFI | 58K | L1 | 1b |
| | | ESLHEDPSQWAELLEEARRLNDAQAPK | | | |
| 164 | 212 | LDEEEFQAYLQAWYEIFNLPNLNDTQWPAFI | 58K | L1 | 1b |
| | | ESLHEDPSQWAELLEEARRLNDAQAPK | | | |
| 165 | 213 | LDEEEFQAYLQAWYEIFNLPNLNDAQWPAFI | 58K | L1 | 1b |
| | | ESLHEDPSQWAELLEEARRLNDAQAPK | | | |
| 166 | 214 | LDEEEFQAYLQAWYEIFNLPNLNDLQWPAFI | 58K | L1 | 1b |
| | | ESLHEDPSQWAELLEEARRLNDAQAPK | | | |
| 167 | 215 | LDEEEFQAYLQAWYEIFNLPNLNDGQWPAFI | 58K | L1 | 1b |
| | | ESLHEDPSQWAELLEEARRLNDAQAPK | | | |
| 168 | 216 | LDEEEFAAYLQAWYEIFNLPNLNDNQWPAFI | 58K | L1 | 1b |
| | | ESLHEDPSQWANLLEEARRLNDAQAPK | | | |
| 169 | 217 | LDEEEFQAYVQAWYEIFNLPNLNDNQWPAFI | 58K | L1 | 1b |
| | | ESLHEDPSQWANLLEEARRLNDAQAPK | | | |
| 170 | 218 | LDEEEFQAYIQAWYEIFNLPNLNDNQWPAFI | 58K | L1 | 1b |
| | | ESLHEDPSQWANLLEEARRLNDAQAPK | | | |
| 171 | 219 | LDEEEFQAYFQAWYEIFNLPNLNDNQWPAFI | 58K | L1 | 1b |
| | | ESLHEDPSQWANLLEEARRLNDAQAPK | | | |
| 172 | 220 | LDEEEFQAYLQAWFEIFNLPNLNDNQWPAFI | 58K | L1 | 1b |
| | | ESLHEDPSQWANLLEEARRLNDAQAPK | | | |
| 173 | 221 | LDEEEFQAYLQAWWEIFNLPNLNDNQWPAFI | 58K | L1 | 1b |
| | | ESLHEDPSQWANLLEEARRLNDAQAPK | | | |
| 174 | 222 | LDEEEFQAYLQAWHEIFNLPNLNDNQWPAFI | 58K | L1 | 1b |
| | | ESLHEDPSQWANLLEEARRLNDAQAPK | | | |
| 175 | 172 | LDEEEFQAYLQAWYEIFNLPNLNDQQWPAFI | 58K | L1 | 1c |
| | | ESLHEDPSQWANLLEEARRLNDAQAPK | | | |
| 176 | 172 | LDEEEFQAYLQAWYEIFNLPNLNDQQWPAFI | 58K | L1 | 1a |
| | | ESLHEDPSQWANLLEEARRLNDAQAPK | | | |
| 177 | 172 | LDEEEFQAYLQAWYEIFNLPNLNDQQWPAFI | 58K | L3 | 1b |
| | | ESLHEDPSQWANLLEEARRLNDAQAPK | | | |
| 178 | 172 | LDEEEFQAYLQAWYEIFNLPNLNDQQWPAFI | 58K | L3 | 1c |
| | | ESLHEDPSQWANLLEEARRLNDAQAPK | | | |
| 179 | 172 | LDEEEFQAYLQAWYEIFNLPNLNDQQWPAFI | 58K | L3 | 1a |
| | | ESLHEDPSQWANLLEEARRLNDAQAPK | | | |
| 180 | 172 | LDEEEFQAYLQAWYEIFNLPNLNDQQWPAFI | 58K | L2 | 1b |
| | | ESLHEDPSQWANLLEEARRLNDAQAPK | | | |
| 181 | 172 | LDEEEFQAYLQAWYEIFNLPNLNDQQWPAFI | 58K | L2 | 1c |
| | | ESLHEDPSQWANLLEEARRLNDAQAPK | | | |
| 182 | 172 | LDEEEFQAYLQAWYEIFNLPNLNDQQWPAFI | 58K | L2 | 1a |
| | | ESLHEDPSQWANLLEEARRLNDAQAPK | | | |
| 183 | 209 | LDEEEFQAYLQAWYEIFQLPNLNDNQWPAFI | 58K | L1 | 1c |
| | | ESLHEDPSQWANLLEEARRLNDAQAPK | | | |
| 184 | 223 | LDEEEFQAYLQAWYEIFNLPQLNDNQWPAFI | 58K | L1 | 1c |
| | | ESLHEDPSQWANLLEEARRLNDAQAPK | | | |
| 185 | 224 | LDEEEFQAYLQAWYEIFNLPNLQDNQWPAFI | 58K | L1 | 1c |
| | | ESLHEDPSQWANLLEEARRLNDAQAPK | | | |
| 186 | 179 | LDEEEFQAYLQAWYEIFNLPNLNDNQWPAFI | 58K | L1 | 1c |
| | | ESLHEDPSQWAQLLEEARRLNDAQAPK | | | |
| 187 | 225 | VDEEEFQAYLQAWYEIFNLPNLNDNQWPAFI | 58K | L1 | 1b |
| | | ESLHEDPSQWANLLEEARRLNDAQAPK | | | |
| 188 | 226 | VDEEEFQAYLQAWYEIFNLPNLNDQQWPAF | 58K | L1 | 1b |
| | | IESLHEDPSQWANLLEEARRLNDAQAPK | | | |
| 189 | 227 | LEEEEFQAYLQAWYEIFNLPNLNDNQWPAFI | 58K | L1 | 1b |
| | | ESLHEDPSQWANLLEEARRLNDAQAPK | | | |
| 190 | 228 | LEEEEFQAYLQAWYEIFNLPNLNDNQWPAFI | 58K | L1 | 1b |
| | | ESLHEEPSQWANLLEEARRLNDAQAPK | | | |
| 191 | 229 | LEEEEFQAYLQAWYEIFNLPNLNDQQWPAFI | 58K | L1 | 1b |
| | | ESLHEEPSQWANLLEEARRLNDAQAPK | | | |
| 192 | 225 | VDEEEFQAYLQAWYEIFNLPNLNDNQWPAFI | 58K | L1 | 1c |
| | | ESLHEDPSQWANLLEEARRLNDAQAPK | | | |
| 193 | 226 | VDEEEFQAYLQAWYEIFNLPNLNDQQWPAFI | 58K | L1 | 1c |
| | | ESLHEDPSQWANLLEEARRLNDAQAPK | | | |
| 194 | 227 | LEEEEFQAYLQAWYEIFNLPNLNDNQWPAFI | 58K | L1 | 1c |
| | | ESLHEDPSQWANLLEEARRLNDAQAPK | | | |
| 195 | 228 | LEEEEFQAYLQAWYEIFNLPNLNDNQWPAFI | 58K | L1 | 1c |
| | | ESLHEEPSQWANLLEEARRLNDAQAPK | | | |
| 196 | 229 | LEEEEFQAYLQAWYEIFNLPNLNDQQWPAFI | 58K | L1 | 1c |
| | | ESLHEEPSQWANLLEEARRLNDAQAPK | | | |
| 197 | 168 | LDEEEFQAYLQAWYEIFNLPNLNDQQWPAFI | 58K | L1 | 1c |
| | | ESLHEDPSQWANLLAEARRLNDAQAPK | | | |
| 198 | 230 | LDEEEFQAYLQAWYEIFNLPNLNDEQWPAFI | 58K | L1 | 1c |
| | | ESLHEDPSQWANLLAEARRLNDAQAPK | | | |
| 199 | 171 | LDEEEFQAYLQAWYEIFNLPNLNDEQWPAFI | 58K | L1 | 1c |
| | | ESLHEDPSQWANLLEEARRLNDAQAPK | | | |
| 200 | 231 | LDEEEFQAYLQAWYEIFNLPNLNDAQWPAFI | 58K | L1 | 1c |
| | | ESLHEDPSQWANLLAEARRLNDAQAPK | | | |
| 201 | 176 | LDEEEFQAYLQAWYEIFNLPNLNDAQWPAFI | 58K | L1 | 1c |
| | | ESLHEDPSQWANLLEEARRLNDAQAPK | | | |
| 202 | 232 | LDEEEFQAYLQAWYEIFNLPNLQDQQWPAFI | 58K | L1 | 1c |
| | | ESLHEDPSQWANLLEEARRLNDAQAPK | | | |
| 203 | 233 | LDEEEFQAYLQAWYEIFNLPNLNDQQWPAFI | 58K | L1 | 1c |
| | | ESLHEDPSQWAQLLAEARRLNDAQAPK | | | |
| 204 | 234 | LDEEEFQAYLQAWYEIFNLPNLNDQQWPAFI | 58K | L1 | 1c |
| | | ESLHEDPSQWANLLAEARQLNDAQAPK | | | |
| 205 | 235 | LDEEEFQAYLQAWYEIFNLPHLNDQQWPAFI | 58K | L1 | 1c |
| | | ESLHEDPSQWANLLEEARRLNDAQAPK | | | |
| 206 | 236 | LDEEEFQAYLQAWYEIFNLPNLNDQQWPAFI | 58K | L1 | 1c |
| | | ESLHEDPSQWANLLAEARRLNEAQAPK | | | |
| 207 | 237 | LDEEEFQAYLQAWYEIFNLPNLNDQQWPAFI | 58K | L1 | 1c |
| | | ESLHEDPSQWANLLEEARRLNEAQAPK | | | |
| 208 | 238 | LDEEEFQAYLQAWYEIFNLPNLNDQQWPAFI | 58K | L1 | 1c |
| | | ESLHEDPSQWANLLAEARQLNEAQAPK | | | |
| 209 | 190 | LDEEEFQAYLQAWYEIFNLPNLTDQQWPAFI | 58K | L1 | 1c |
| | | ESLHEDPSQWANLLEEARRLNDAQAPK | | | |
| 210 | 192 | LDEEEFQAYLQAWYEIFNLPNLTDQQWPAFI | 58K | L1 | 1c |
| | | ESLHEDPSQWAQLLEEARRLNDAQAPK | | | |
| 211 | 239 | LDEEEFQAYLQAWYEIFNLPNLDDAQWPAFI | 58K | L1 | 1c |
| | | ESLHEDPSQWANLLEEARRLNDAQAPK | | | |
| 212 | 240 | LDEEEFQAYLQAWYEIFNLPNLNDQQWPAFI | 58K | L1 | 1c |
| | | ESLHEDPSQWANLLEEARQLNDAQAPK | | | |
| 213 | 241 | LDEEEFQAYLQAWYEIFNLPNLTDAQWPAFI | 58K | L1 | 1c |
| | | ESLHEDPSQWANLLEEARRLNDAQAPK | | | |
| 214 | 242 | LDEEEFQAYLQAWYEIFNLPNLTDAQWPAFI | 58K | L1 | 1c |
| | | ESLHEDPSQWAQLLEEARRLNDAQAPK | | | |
| 215 | 243 | LDEEEFQAYLQAWYEIFNLPNLDDQQWPAFI | 58K | L1 | 1c |
| | | ESLHEDPSQWANLLAEARRLNDAQAPK | | | |
| 216 | 244 | LDEEEFQAYLQAWYEIFNLPNLDDQQWPAFI | 58K | L1 | 1c |
| | | ESLHEDPSQWANLLEEARRLNDAQAPK | | | |
| 217 | 245 | LEEEEFQAYLQAWYEIFNLPNLNDAQWPAFI | 58K | L1 | 1c |
| | | ESLHEDPSQWANLLEEARRLNDAQAPK | | | |
| 218 | 246 | LQEEEFQAYLQAWYEIFNLPNLNDAQWPAFI | 58K | L1 | 1c |
| | | ESLHEDPSQWANLLEEARRLNDAQAPK | | | |
| 219 | 247 | LEEEEFQAYLQAWYEIFNLPHLNDAQWPAFI | 58K | L1 | 1c |
| | | ESLHEDPSQWANLLEEARRLNDAQAPK | | | |
| 220 | 248 | LEEEEFQAYLQAWYEIFNLPNLTDAQWPAFI | 58K | L1 | 1c |
| | | ESLHEDPSQWANLLEEARRLNDAQAPK | | | |
| 221 | 249 | LEEEEFQAYLQAWYEIFNLPNLNDQQWPAFI | 58K | L1 | 1c |
| | | ESLHEDPSQWANLLEEARRLNDAQAPK | | | |
| 222 | 250 | LEEEEFQAYLQAWYEIFNLPNLNDYQWPAFI | 58K | L1 | 1c |
| | | ESLHEDPSQWANLLEEARRLNDAQAPK | | | |
| 223 | 251 | LEEEEFQAYLQAWYEIFNLPNLNDLQWPAFI | 58K | L1 | 1c |
| | | ESLHEDPSQWANLLEEARRLNDAQAPK | | | |
| 224 | 252 | LEEEEFQAYLQAWYEIFNLPNLNDAQWPAFI | 58K | L1 | 1c |
| | | ESLHEDPSQWASLLEEARRLNDAQAPK | | | |
| 225 | 253 | LEEEEFQAYLQAWYEIFNLPNLNDAQWPAFI | 58K | L1 | 1c |
| | | ESLHEDPSQWAELLEEARRLNDAQAPK | | | |
| 226 | 254 | LEEEEFQAYLQAWYEIFNLPNLNDAQWPAFI | 58K | L1 | 1c |
| | | ESLHEDPSQWANLLEEARRLNEAQAPK | | | |
| 227 | 255 | LEEEEFQAYLQAWYEIFNLPNLNDAQWPAFI | 58K | L1 | 1c |
| | | ESLHEDPSQWANLLEEARRLNTAQAPK | | | |
| 228 | 256 | LEEEEFQAYLQAWYEIFNLPNLNDAQWPAFI | 58K | L1 | 1c |
| | | ESLHEDPSQWANLLEEARRLNDIQAPK | | | |
| 229 | 257 | LEEEEFQAYLQAWYEIFNLPHLTDQQWPAFI | 58K | L1 | 1c |
| | | ESLHEEPSQWAELLEEARRLNEAQAPK | | | |
| 230 | 258 | LEEEEFQAYLQAWYEIFNLPHLTDQQWPAFI | 58K | L1 | 1c |
| | | ESLHEEPSQWAQLLEEARRLNEAQAPK | | | |
| 231 | 259 | LEEEEFQAYLQAWYEIFNLPNLTDQQWPAFI | 58K | L1 | 1c |
| | | ESLHEDPSQWAELLEEARRLNDAQAPK | | | |
| 232 | 260 | LEEEEFQAYLQAWYEIFNLPNLTDAQWPAFI | 58K | L1 | 1c |
| | | ESLHEDPSQWAELLEEARRLNDAQAPK | | | |
| 233 | 261 | LEEEEFQAYLQAWYEIFNLPNLTDAQWPAFI | 58K | L1 | 1c |
| | | ESLHEEPSQWAELLEEARRLNDAQAPK | | | |
| 234 | 262 | LEEEEFQAYLQAWYEIFNLPNLTDYQWPAFI | 58K | L1 | 1c |
| | | ESLHEEPSQWANLLEEARRLNDAQAPK | | | |
| 235 | 263 | LEEEEFQAYLQAWYEIFNLPNLTDQQWPAFI | 58K | L1 | 1c |
| | | ESLHEDPSQWAQLLEEARRLNEAQAPK | | | |
| 236 | 264 | LEEEEFQAYLQAWYEIFNLPNLNDQQWPAFI | 58K | L1 | 1c |
| | | ESLHEDPSQWAQLLEEARRLNEAQAPK | | | |
| 237 | 265 | LEEEEFQAYLQAWYEIFNLPNLTDAQWPAFI | 58K | L1 | 1c |
| | | ESLHEDPSQWAQLLEEARRLNEAQAPK | | | |
| 238 | 266 | LEEEEFQAYLQAWYEIFNLPNLTDYQWPAFI | 58K | L1 | 1c |
| | | ESLHEDPSQWANLLEEARRLNEAQAPK | | | |
| 239 | 267 | LEEEEFQAYLQAWYEIFNLPHLNDQQWPAFI | 58K | L1 | 1c |
| | | ESLHEEPSQWAQLLEEARRLNEAQAPK | | | |
| 240 | 268 | LEEEEFQAYLQAWYEIFNLPHLNDQQWPAFI | 58K | L1 | 1c |
| | | ESLHEEPSQWAQLLEEARRLNDAQAPK | | | |
| 241 | 269 | LEEEEFQAYLQAWYEIFNLPHLNDQQWPAFI | 58K | L1 | 1c |
| | | ESLHEEPSQWANLLEEARRLNEAQAPK | | | |
| 242 | 270 | LEEEEFQAYLQAWYEIFNLPHLNDQQWPAFI | 58K | L1 | 1c |
| | | ESLHEDPSQWAQLLEEARRLNEAQAPK | | | |
| 243 | 271 | LEEEEFQAYLQAWYEIFNLPHLNDQQWPAFI | 58K | L1 | 1c |
| | | ESLHEDPSQWAQLLEEARRLNDAQAPK | | | |
| 244 | 272 | LEEEEFQAYLQAWYEIFTLPHLTDQQWPAFI | 58K | L1 | 1c |
| | | ESLHEEPSQWAELLEEARRLNEAQAPK | | | |
| 245 | 273 | LEEEEFQAYLQAWYEIFTLPHLTDQQWPAFI | 58K | L1 | 1c |
| | | ESLHEEPSQWAQLLEEARRLNEAQAPK | | | |
| 246 | 274 | VDQEEFQAYLQAWYEIFNLPCLNDNQWPAF | 58K | L1 | 1b |
| | | IESLHEDPSQWDNLLEEARRLNDAQCPK | | | |
| 247 | 275 | LDNEEFQAYLQAWYEIFNLPCLNDNQWPAFI | 58K | L1 | 1b |
| | | ESLHEDPSQWDNLLEEARRLNDAQCPK | | | |
| 248 | 276 | LDNDEFILYLQAWYEIFNLPCLNDNQWPAFI | 58K | L1 | 1b |
| | | ESLHDDPSQWANLLAEARRLNDAQCPK | | | |
| 249 | 277 | LDNDEFILYLQAWYEIFNLPCLNDNQWPAFI | 58K | L1 | 1b |
| | | ESLHDDPSQWANLLEEARRLNDAQCPK | | | |
| 250 | 278 | LDNDEFQLYLQAWYEIFNLPCLNDNQWPAFI | 58K | L1 | 1b |
| | | ESLHDDPSQWANLLEEARRLNDAQCPK | | | |
| 251 | 279 | LDEEEFQAYLQAWYEIFNLPCLNDQQWPAFI | 58K | L1 | 1c |
| | | ESLHEDPSQWANLLEEARRLNDAQCPK | | | |
| 252 | 280 | LDEEEFQAYLQAWYEIFNLPCLTDSQWPAFI | 58K | L1 | 1c |
| | | ESLHEDPSQWANLLEEARRLNDAQCPK | | | |

Binding motif (BM) is highlighted in bold in the sequences of Table 4.

TABLE 5
Technical and analytical information on the
hFGF23 antagonistic polypeptide derivatives

| | | | LCMS |
|---|---|---|---|
| Chem. | Sumformula | m/z found | Method |
| 3 | C₃₀₉H₄₄₈N₈₄O₉₅ | m/7: 980.8 (avg) | M111 |
| 4 | C₃₄₅H₅₁₄N₈₈O₁₀₇ | m/7: 1087.5 (avg) | M111 |
| 5 | C₃₄₉H₅₂₂N₈₄O₁₀₄ | m/7: 1080.6 (avg) | M111 |
| 6 | C₃₄₆H₅₁₆N₈₈O₁₀₅ | m/8: 949.4 (avg) | M111 |
| 7 | C₃₅₀H₅₂₂N₈₄O₁₀₆ | m/7: 1086.9 (avg) | M111 |
| 8 | C₃₄₇H₅₁₆N₈₈O₁₀₇ | m/7: 1091.1 (avg) | M111 |
| 9 | C₃₄₉H₅₁₇N₈₅O₁₀₉ | m/7: 1093.6 (avg) | M111 |
| 10 | C₃₄₈H₅₁₆N₈₄O₁₀₉ | m/7: 1089.7 (avg) | M111 |
| 11 | C₃₅₀H₅₂₂N₈₄O₁₀₇ | m/7: 1089.4 (avg) | M111 |
| 12 | C₃₄₉H₅₂₀N₈₄O₁₀₇ | m/7: 1087.3 (avg) | M111 |
| 13 | C₃₄₉H₅₂₀N₈₄O₁₀₆ | m/7: 1085.1 (avg) | M111 |
| 14 | C₃₄₆H₅₁₆N₈₄O₁₀₄ | m/7: 1074.8 (avg) | M111 |
| 15 | C₃₄₇H₅₁₈N₈₄O₁₀₄ | m/7: 1076.7 (avg) | M111 |
| 16 | C₃₄₈H₅₁₈N₈₄O₁₀₆ | m/7: 1083.0 (avg) | M111 |
| 17 | C₃₄₆H₅₁₃N₈₅O₁₀₇ | m/7: 1083.0 (avg) | M111 |
| 18 | C₃₄₈H₅₁₈N₈₄O₁₀₆ | m/7: 1082.9 (avg) | M111 |
| 19 | C₃₄₇H₅₁₈N₈₄O₁₀₅ | m/7: 1078.9 (avg) | M111 |
| 20 | C₃₄₇H₅₁₇N₈₅O₁₀₅ | m/7: 1080.7 (avg) | M111 |
| 21 | C₃₄₉H₅₁₈N₈₆O₁₀₄ | m/7: 1084.0 (avg) | M111 |
| 22 | C₃₄₉H₅₂₃N₈₇O₁₀₄ | m/7: 1086.7 (avg) | M111 |
| 23 | C₃₄₆H₅₁₆N₈₄O₁₀₅ | m/7: 1076.8 (avg) | M111 |
| 24 | C₃₄₇H₅₁₈N₈₄O₁₀₄ | m/7: 1076.6 (avg) | M111 |
| 25 | C₃₄₆H₅₁₃N₈₅O₁₀₇ | m/7: 1082.9 (avg) | M111 |
| 26 | C₃₄₅H₅₁₁N₈₅O₁₀₇ | m/7: 1081.0 (avg) | M111 |
| 27 | C₃₄₇H₅₁₆N₈₄O₁₀₆ | m/7: 1080.9 (avg) | M111 |
| 28 | C₃₄₄H₅₁₀N₈₄O₁₀₆ | m/7: 1074.8 (avg) | M111 |
| 29 | C₃₄₇H₅₁₇N₈₇O₁₀₆ | m/7: 1087.0 (avg) | M111 |
| 30 | C₃₄₃H₅₀₈N₈₄O₁₀₆ | m/7: 1072.8 (avg) | M111 |
| 31 | C₃₄₆H₅₁₂N₈₄O₁₀₆ | m/7: 1078.6 (avg) | M111 |
| 32 | C₃₄₇H₅₁₂N₈₆O₁₀₆ | m/7: 1084.3 (avg) | M111 |
| 33 | C₃₄₆H₅₁₄N₈₄O₁₀₆ | m/7: 1078.9 (avg) | M111 |
| 34 | C₃₅₆H₅₂₄N₈₄O₁₀₅ | m/7: 1095.2 (avg) | M111 |
| 35 | C₃₅₄H₅₂₃N₈₃O₁₀₆ | m/7: 1091.9 (avg) | M111 |
| 36 | C₃₄₂H₅₁₆N₈₆O₁₀₈ | m/7: 1080.8 (avg) | M111 |
| 37 | C₃₅₂H₅₂₄N₈₄O₁₀₈ | m/7: 1095.1 (avg) | M111 |
| 38 | C₃₅₈H₅₂₆N₈₄O₁₀₇ | m/7: 1103.4 (avg) | M111 |
| 39 | C₃₅₇H₅₂₆N₈₄O₁₀₅ | m/7: 1097.1 (avg) | M111 |
| 40 | C₃₄₂H₅₀₆N₈₄O₁₀₆ | m/7: 1070.9 (avg) | M111 |
| 41 | C₃₄₃H₅₀₈N₈₄O₁₀₆ | m/7: 1072.9 (avg) | M111 |
| 42 | C₃₄₄H₅₁₃N₈₅O₁₀₄ | m/7: 1072.7 (avg) | M111 |
| 43 | C₃₄₃H₅₁₁N₈₅O₁₀₄ | m/7: 1070.7 (avg) | M111 |
| 44 | C₃₄₄H₅₁₁N₈₅O₁₀₅ | m/7: 1074.7 (avg) | M111 |
| 45 | C₃₄₄H₅₁₁N₈₅O₁₀₅ | m/7: 1074.7 (avg) | M111 |
| 46 | C₃₄₃H₅₀₉N₈₅O₁₀₅ | m/7: 1072.7 (avg) | M111 |
| 47 | C₃₄₅H₅₁₅N₈₅O₁₀₄ | m/7: 1074.7 (avg) | M111 |
| 48 | C₃₄₅H₅₁₅N₈₅O₁₀₄ | m/7: 1074.7 (avg) | M111 |
| 49 | C₃₄₄H₅₁₃N₈₅O₁₀₄ | m/7: 1072.7 (avg) | M111 |
| 50 | C₃₄₄H₅₁₃N₈₅O₁₀₄ | m/7: 1072.7 (avg) | M111 |
| 51 | C₃₄₆H₅₁₄N₈₆O₁₀₃ | m/7: 1076.0 (avg) | M111 |
| 52 | C₃₄₄H₅₁₃N₈₅O₁₀₃ | m/7: 1070.5 (avg) | M111 |
| 53 | C₃₄₁H₅₁₁N₈₇O₁₀₃ | m/7: 1069.0 (avg) | M111 |
| 54 | C₃₄₉H₅₁₉N₈₅O₁₀₄ | m/7: 1082.2 (avg) | M111 |
| 55 | C₃₄₇H₅₁₈N₈₄O₁₀₄ | m/7: 1076.6 (avg) | M111 |
| 56 | C₃₄₄H₅₁₆N₈₆O₁₀₄ | m/7: 1075.1 (avg) | M111 |
| 57 | C₃₅₀H₅₂₃N₈₅O₁₀₂ | m/7: 1079.9 (avg) | M111 |
| 58 | C₃₄₄H₅₀₈N₈₄O₁₀₈ | m/7: 1079.1 (avg) | M111 |
| 59 | C₃₄₅H₅₁₀N₈₄O₁₀₈ | m/7: 1081.2 (avg) | M111 |
| 60 | C₃₄₆H₅₁₅N₈₅O₁₀₆ | m/7: 1081.0 (avg) | M111 |
| 61 | C₃₄₆H₅₁₅N₈₅O₁₀₆ | m/7: 1081.0 (avg) | M111 |
| 62 | C₃₄₅H₅₁₃N₈₅O₁₀₆ | m/7: 1079.0 (avg) | M111 |
| 63 | C₃₄₆H₅₁₃N₈₅O₁₀₇ | m/7: 1083.0 (avg) | M111 |
| 64 | C₃₄₆H₅₁₃N₈₅O₁₀₇ | m/7: 1083.0 (avg) | M111 |
| 65 | C₃₄₅H₅₁₁N₈₅O₁₀₇ | m/7: 1081.0 (avg) | M111 |
| 66 | C₃₄₇H₅₁₇N₈₅O₁₀₆ | m/7: 1083.0 (avg) | M111 |
| 67 | C₃₄₇H₅₁₇N₈₅O₁₀₆ | m/7: 1083.1 (avg) | M111 |
| 68 | C₃₄₈H₅₁₆N₈₆O₁₀₅ | m/7: 1084.3 (avg) | M111 |
| 69 | C₃₄₃H₅₁₃N₈₇O₁₀₅ | m/7: 1077.3 (avg) | M111 |
| 70 | C₃₅₁H₅₂₁N₈₅O₁₀₆ | m/7: 1090.4 (avg) | M111 |
| 71 | C₃₄₅H₅₁₁N₈₅O₁₀₇ | m/7: 1081.0 (avg) | M111 |
| 72 | C₃₄₇H₅₁₈N₈₈O₁₀₅ | m/7: 1086.8 (avg) | M111 |
| 73 | C₃₄₅H₅₁₁N₈₅O₁₀₇ | m/7: 1081.0 (avg) | M111 |
| 74 | C₃₄₅H₅₁₀N₈₄O₁₀₈ | m/7: 1081.1 (avg) | M111 |
| 75 | C₃₄₅H₅₁₁N₈₅O₁₀₇ | m/7: 1081.0 (avg) | M111 |
| 76 | C₃₄₆H₅₁₅N₈₅O₁₀₅ | m/7: 1078.7 (avg) | M111 |
| 77 | C₃₄₅H₅₁₃N₈₅O₁₀₆ | m/7: 1079.0 (avg) | M111 |
| 78 | C₃₄₅H₅₁₂N₈₄O₁₀₇ | m/7: 1079.2 (avg) | M111 |
| 79 | C₃₄₅H₅₁₀N₈₄O₁₀₈ | m/7: 1081.1 (avg) | M111 |
| 80 | C₃₄₆H₅₁₃N₈₅O₁₀₇ | m/7: 1083.0 (avg) | M111 |
| 81 | C₃₄₅H₅₁₄N₈₄O₁₀₅ | m/7: 1074.9 (avg) | M111 |
| 82 | C₃₄₅H₅₁₃N₈₅O₁₀₆ | m/7: 1079.0 (avg) | M111 |
| 83 | C₃₄₆H₅₁₃N₈₅O₁₀₇ | m/7: 1083.0 (avg) | M111 |
| 84 | C₃₄₆H₅₁₂N₈₄O₁₀₈ | m/7: 1083.1 (avg) | M111 |
| 85 | C₃₄₅H₅₁₅N₈₅O₁₀₄ | m/7: 1074.7 (avg) | M111 |
| 86 | C₃₄₇H₅₁₇N₈₅O₁₀₅ | m/7: 1080.7 (avg) | M111 |
| 87 | C₃₄₆H₅₁₇N₈₅O₁₀₄ | m/7: 1076.7 (avg) | M111 |
| 88 | C₃₄₇H₅₁₅N₈₅O₁₀₇ | m/7: 1085.0 (avg) | M111 |
| 89 | C₃₄₇H₅₁₄N₈₄O₁₀₈ | m/7: 1085.1 (avg) | M111 |
| 90 | C₃₄₇H₅₁₇N₈₅O₁₀₅ | m/7: 1080.7 (avg) | M111 |
| 91 | C₃₄₆H₅₁₇N₈₅O₁₀₄ | m/7: 1076.8 (avg) | M111 |
| 92 | C₃₄₈H₅₁₉N₈₅O₁₀₅ | m/7: 1082.7 (avg) | M111 |
| 93 | C₃₄₈H₅₁₈N₈₄O₁₀₆ | m/7: 1082.9 (avg) | M111 |
| 94 | C₃₄₇H₅₁₉N₈₅O₁₀₄ | m/7: 1078.7 (avg) | M111 |
| 95 | C₃₄₆H₅₁₆N₈₄O₁₀₅ | m/7: 1076.9 (avg) | M111 |
| 96 | C₃₄₃H₅₀₇N₈₅O₁₀₇ | m/7: 1077.0 (avg) | M111 |
| 97 | C₃₄₇H₅₁₉N₈₇O₁₀₄ | m/7: 1082.8 (avg) | M111 |
| 98 | C₃₄₇H₅₁₉N₈₇O₁₀₄ | m/7: 1082.7 (avg) | M111 |
| 99 | C₃₄₈H₅₂₀N₈₈O₁₀₅ | m/7: 1088.9 (avg) | M111 |
| 100 | C₃₄₉H₅₁₅N₈₅O₁₁₁ | m/7: 1097.5 (avg) | M111 |
| 101 | C₃₄₇H₅₁₃N₈₅O₁₀₉ | m/7: 1089.3 (avg) | M111 |
| 102 | C₃₄₆H₅₁₇N₈₇O₁₀₄ | m/7: 1080.7 (avg) | M111 |
| 103 | C₃₄₃H₅₁₁N₈₇O₁₀₄ | m/7: 1074.7 (avg) | M111 |
| 104 | C₃₄₄H₅₁₁N₈₅O₁₀₆ | m/7: 1077.6 (avg) | M111 |
| 105 | C₃₄₀H₅₀₄N₈₄O₁₀₅ | m/6: 1242.7 (avg) | M111 |
| 106 | C₃₄₉H₅₁₈N₈₄O₁₀₈ | m/6: 1269.969 (mono) | M64 |
| 107 | C₃₄₉H₅₁₆N₈₄O₁₁₀ | m/6: 1274.967 (mono) | M64 |
| 108 | C₃₄₇H₅₁₅N₈₅O₁₀₇ | m/7: 1085.8 (avg) | M111 |
| 109 | C₃₄₉H₅₁₇N₈₅O₁₀₉ | m/7: 1093.8 (avg) | M111 |
| 110 | C₃₅₂H₅₂₃N₈₅O₁₀₉ | m/6: 1281.82 (mono) | M64 |
| 111 | C₃₄₈H₅₁₂N₈₂O₁₁₁ | m/7: 1090.2 (avg) | M111 |
| 112 | C₃₄₉H₅₁₄N₈₂O₁₁₂ | m/7: 1094.2 (avg) | M111 |
| 113 | C₃₄₈H₅₁₃N₈₁O₁₁₂ | m/7: 1089.8 (avg) | M111 |
| 114 | C₃₅₁H₅₁₇N₈₅O₁₀₉ | m/7: 1096.6 (avg) | M111 |
| 115 | C₃₄₉H₅₁₆N₈₄O₁₀₉ | m/7: 1091.8 (avg) | M111 |
| 116 | C₃₄₁H₅₁₁N₈₃O₁₁₀ | m/7: 1077.4 (avg) | M111 |
| 117 | C₃₅₁H₅₁₉N₈₅O₁₁₀ | m/7: 1099.8 (avg) | M111 |
| 118 | C₃₄₈H₅₁₆N₈₄O₁₀₈ | m/7: 1087.6 (avg) | M111 |
| 119 | C₃₄₈H₅₁₆N₈₄O₁₀₈ | m/7: 1087.6 (avg) | M111 |
| 120 | C₃₄₈H₅₁₆N₈₄O₁₀₈ | m/7: 1087.6 (avg) | M111 |
| 121 | C₃₄₈H₅₁₆N₈₄O₁₀₈ | m/7: 1087.7 (avg) | M111 |
| 122 | C₃₄₉H₅₁₅N₈₃O₁₁₁ | m/7: 1094.0 (avg) | M111 |
| 123 | C₃₅₀H₅₁₇N₈₃O₁₁₁ | m/7: 1095.8 (avg) | M111 |
| 124 | C₃₅₀H₅₁₈N₈₄O₁₁₀ | m/7: 1095.5 (avg) | M111 |
| 125 | C₃₄₈H₅₁₅N₈₃O₁₁₀ | m/7: 1090.1 (avg) | M111 |
| 126 | C₃₄₉H₅₁₇N₈₃O₁₁₀ | m/7: 1092.2 (avg) | M111 |
| 127 | C₃₅₁H₅₁₇N₈₅O₁₀₉ | m/7: 1097.4 (avg) | M111 |
| 128 | C₃₄₈H₅₁₅N₈₃O₁₀₉ | m/7: 1088.0 (avg) | M111 |
| 129 | C₃₅₁H₅₂₁N₈₃O₁₀₉ | m/7: 1093.7 (avg) | M111 |
| 130 | C₃₅₀H₅₁₇N₈₃O₁₁₁ | m/7: 1095.6 (avg) | M111 |
| 131 | C₃₅₀H₅₁₈N₈₄O₁₁₀ | m/7: 1095.4 (avg) | M111 |
| 132 | C₃₅₁H₅₁₈N₈₂O₁₁₂ | m/7: 1097.6 (avg) | M111 |
| 133 | C₃₅₁H₅₁₉N₈₃O₁₁₁ | m/7: 1097.5 (avg) | M111 |
| 134 | C₃₅₁H₅₁₉N₈₃O₁₁₁ | m/7: 1097.4 (avg) | M111 |
| 135 | C₃₅₁H₅₂₀N₈₄O₁₁₀ | m/7: 1097.3 (avg) | M111 |
| 136 | C₃₅₄H₅₁₉N₈₃O₁₁₀ | m/7: 1100.5 (avg) | M111 |
| 137 | C₃₅₀H₅₁₈N₈₂O₁₁₁ | m/7: 1093.6 (avg) | M111 |
| 138 | C₃₅₀H₅₁₉N₈₃O₁₁₀ | m/7: 1093.5 (avg) | M111 |
| 139 | C₃₅₀H₅₁₈N₈₂O₁₁₁ | m/7: 1093.5 (avg) | M111 |
| 140 | C₃₅₁H₅₁₉N₈₁O₁₁₂ | m/7: 1095.8 (avg) | M111 |
| 141 | C₃₅₁H₅₂₀N₈₂O₁₁₁ | m/7: 1095.6 (avg) | M111 |
| 142 | C₃₅₀H₅₁₉N₈₃O₁₁₀ | m/7: 1093.5 (avg) | M111 |
| 143 | C₃₅₁H₅₂₀N₈₂O₁₁₁ | m/7: 1095.6 (avg) | M111 |
| 144 | C₃₅₁H₅₂₁N₈₃O₁₁₀ | m/7: 1095.5 (avg) | M111 |
| 145 | C₃₅₀H₅₁₈N₈₄O₁₁₀ | m/7: 1095.7 (avg) | M111 |
| 146 | C₃₅₁H₅₁₉N₈₃O₁₁₁ | m/7: 1097.7 (avg) | M111 |
| 147 | C₃₅₁H₅₂₀N₈₄O₁₁₀ | m/7: 1097.4 (avg) | M111 |
| 148 | C₃₅₁H₅₁₉N₈₃O₁₁₁ | m/7: 1097.7 (avg) | M111 |
| 149 | C₃₅₂H₅₂₀N₈₂O₁₁₂ | m/7: 1099.6 (avg) | M111 |
| 150 | C₃₅₂H₅₂₁N₈₃O₁₁₁ | m/7: 1099.5 (avg) | M111 |
| 151 | C₃₅₁H₅₂₀N₈₄O₁₁₀ | m/7: 1097.5 (avg) | M111 |
| 152 | C₃₅₂H₅₂₁N₈₃O₁₁₁ | m/7: 1099.6 (avg) | M111 |
| 153 | C₃₅₀H₅₁₇N₈₃O₁₁₁ | m/7: 1095.7 (avg) | M111 |
| 154 | C₃₄₈H₅₁₅N₈₃O₁₁₀ | m/7: 1089.5 (avg) | M111 |
| 155 | C₃₄₉H₅₁₇N₈₃O₁₁₀ | m/7: 1091.7 (avg) | M111 |
| 156 | C₃₅₀H₅₁₉N₈₃O₁₁₀ | m/7: 1093.7 (avg) | M111 |
| 157 | C₃₄₉H₅₁₇N₈₃O₁₀₉ | m/7: 1089.4 (avg) | M111 |
| 158 | C₃₅₂H₅₂₃N₈₃O₁₀₉ | m/7: 1095.3 (avg) | M111 |
| 159 | C₃₄₈H₅₁₅N₈₃O₁₀₉ | m/7: 1087.4 (avg) | M111 |
| 160 | C₃₅₀H₅₁₆N₈₂O₁₁₂ | m/7: 1095.7 (avg) | M111 |
| 161 | C₃₅₀H₅₁₈N₈₄O₁₁₀ | m/7: 1095.4 (avg) | M111 |
| 162 | C₃₄₈H₅₁₅N₈₃O₁₁₀ | m/7: 1089.4 (avg) | M111 |
| 163 | C₃₄₉H₅₁₆N₈₂O₁₁₁ | m/7: 1091.8 (avg) | M111 |
| 164 | C₃₅₀H₅₁₈N₈₂O₁₁₁ | m/7: 1093.6 (avg) | M111 |
| 165 | C₃₄₉H₅₁₆N₈₂O₁₁₀ | m/7: 1089.3 (avg) | M111 |
| 166 | C₃₅₂H₅₂₂N₈₂O₁₁₀ | m/7: 1095.5 (avg) | M111 |
| 167 | C₃₄₈H₅₁₄N₈₂O₁₁₀ | m/7: 1087.6 (avg) | M111 |
| 168 | C₃₄₇H₅₁₃N₈₃O₁₀₉ | m/7: 1085.1 (avg) | M111 |
| 169 | C₃₄₈H₅₁₄N₈₄O₁₁₀ | m/7: 1091.5 (avg) | M111 |
| 170 | C₃₄₉H₅₁₆N₈₄O₁₁₀ | m/7: 1093.5 (avg) | M111 |
| 171 | C₃₅₂H₅₁₄N₈₄O₁₁₀ | m/7: 1098.3 (avg) | M111 |
| 172 | C₃₄₉H₅₁₆N₈₄O₁₀₉ | m/7: 1091.1 (avg) | M111 |
| 173 | C₃₅₁H₅₁₇N₈₅O₁₀₉ | m/7: 1096.8 (avg) | M111 |
| 174 | C₃₄₆H₅₁₄N₈₆O₁₀₉ | m/7: 1089.7 (avg) | M111 |
| 175 | C₃₅₂H₅₂₂N₈₄O₁₁₀ | m/7: 1099.5 (avg) | M111 |
| 176 | C₃₄₈H₅₁₄N₈₄O₁₁₀ | m/7: 1091.4 (avg) | M111 |
| 177 | C₃₄₄H₅₀₇N₈₃O₁₀₇ | m/7: 1074.7 (avg) | M111 |
| 178 | C₃₄₆H₅₁₁N₈₃O₁₀₇ | m/7: 1078.7 (avg) | M111 |
| 179 | C₃₄₂H₅₀₃N₈₃O₁₀₇ | m/7: 1070.5 (avg) | M111 |
| 180 | C₃₆₂H₅₄₀N₈₆O₁₁₆ | m/7: 1136.9 (avg) | M111 |
| 181 | C₃₆₄H₅₄₄N₈₆O₁₁₆ | m/7: 1140.7 (avg) | M111 |
| 182 | C₃₆₀H₅₃₆N₈₆O₁₁₆ | m/7: 1133.0 (avg) | M111 |
| 183 | C₃₅₂H₅₂₂N₈₄O₁₁₀ | m/7: 1099.5 (avg) | M111 |
| 184 | C₃₅₂H₅₂₂N₈₄O₁₁₀ | m/7: 1099.5 (avg) | M111 |
| 185 | C₃₅₂H₅₂₂N₈₄O₁₁₀ | m/7: 1099.4 (avg) | M111 |
| 186 | C₃₅₂H₅₂₂N₈₄O₁₁₀ | m/7: 1099.4 (avg) | M111 |
| 187 | C₃₄₈H₅₁₄N₈₄O₁₁₀ | m/7: 1091.4 (avg) | M111 |
| 188 | C₃₄₉H₅₁₆N₈₄O₁₁₀ | m/7: 1093.5 (avg) | M111 |
| 189 | C₃₅₀H₅₁₈N₈₄O₁₁₀ | m/7: 1095.4 (avg) | M111 |
| 190 | C₃₅₁H₅₂₀N₈₄O₁₁₀ | m/7: 1097.5 (avg) | M111 |
| 191 | C₃₅₂H₅₂₂N₈₄O₁₁₀ | m/7: 1099.4 (avg) | M111 |
| 192 | C₃₅₀H₅₁₈N₈₄O₁₁₀ | m/7: 1095.4 (avg) | M111 |
| 193 | C₃₅₁H₅₂₀N₈₄O₁₁₀ | m/7: 1097.5 (avg) | M111 |
| 194 | C₃₅₂H₅₂₂N₈₄O₁₁₀ | m/7: 1099.4 (avg) | M111 |
| 195 | C₃₅₃H₅₂₄N₈₄O₁₁₀ | m/7: 1101.5 (avg) | M111 |
| 196 | C₃₅₄H₅₂₆N₈₄O₁₁₀ | m/7: 1103.4 (avg) | M111 |
| 197 | C₃₅₀H₅₂₀N₈₄O₁₀₈ | m/6: 1272.297 (mono) | M64 |
| 198 | C₃₅₀H₅₁₉N₈₃O₁₀₉ | m/6: 1272.458 (mono) | M64 |
| 199 | C₃₅₂H₅₂₁N₈₃O₁₁₁ | m/5: 1538.34 (mono) | M64 |
| 200 | C₃₄₈H₅₁₇N₈₃O₁₀₇ | m/6: 1262.8 (mono) | M64 |
| 201 | C₃₅₀H₅₁₉N₈₃O₁₀₉ | m/6: 1272.458 (mono) | M64 |
| 202 | C₃₅₃H₅₂₄N₈₄O₁₁₀ | m/6: 1284.307 (mono) | M64 |
| 203 | C₃₅₁H₅₂₂N₈₄O₁₀₈ | m/6: 1274.633 (mono) | M64 |
| 204 | C₃₄₉H₅₁₆N₈₂O₁₀₉ | m/6: 1267.617 (mono) | M64 |
| 205 | C₃₅₄H₅₂₃N₈₅O₁₀₉ | m/6: 1285.802 (mono) | M64 |
| 206 | C₃₅₁H₅₂₂N₈₄O₁₀₈ | m/6: 1274.642 (mono) | M64 |
| 207 | C₃₅₃H₅₂₄N₈₄O₁₁₀ | m/6: 1284.305 (mono) | M64 |
| 208 | C₃₅₀H₅₁₈N₈₂O₁₀₉ | m/5: 1523.761 (mono) | M64 |
| 209 | C₃₅₂H₅₂₃N₈₃O₁₁₀ | m/5: 1535.63 (mono) | M64 |
| 210 | C₃₅₃H₅₂₅N₈₃O₁₁₀ | m/6: 1282.137 (mono) | M64 |
| 211 | C₃₅₀H₅₁₈N₈₂O₁₁₀ | m/6: 1272.64 (mono) | M64 |
| 212 | C₃₅₁H₅₁₈N₈₂O₁₁₁ | m/5: 1532.57 (mono) | M64 |
| 213 | C₃₅₀H₅₂₀N₈₂O₁₀₉ | m/6: 1270.306 (mono) | M64 |
| 214 | C₃₅₁H₅₂₂N₈₂O₁₀₉ | m/6: 1272.656 (mono) | M64 |
| 215 | C₃₅₀H₅₁₉N₈₃O₁₀₉ | m/7: 1090.853 (mono) | M64 |
| 216 | C₃₅₂H₅₂₁N₈₃O₁₁₁ | m/7: 1100.0 (avg) | M111 |
| 217 | C₃₅₁H₅₂₁N₈₃O₁₀₉ | m/8: 957.8 (avg) | M111 |
| 218 | C₃₅₁H₅₂₂N₈₄O₁₀₈ | m/7: 1093.7 (avg) | M111 |
| 219 | C₃₅₃H₅₂₂N₈₄O₁₀₈ | m/7: 1097.1 (avg) | M111 |
| 220 | C₃₅₁H₅₂₂N₈₂O₁₀₉ | m/7: 1091.5 (avg) | M111 |
| 221 | C₃₅₃H₅₂₄N₈₄O₁₁₀ | m/7: 1101.7 (avg) | M111 |
| 222 | C₃₅₇H₅₂₅N₈₃O₁₁₀ | m/7: 1107.0 (avg) | M111 |
| 223 | C₃₅₄H₅₂₇N₈₃O₁₀₉ | m/7: 1099.8 (avg) | M111 |
| 224 | C₃₅₀H₅₂₀N₈₂O₁₀₉ | m/7: 1089.8 (avg) | M111 |
| 225 | C₃₅₂H₅₂₂N₈₂O₁₁₀ | m/8: 959.2 (avg) | M111 |
| 226 | C₃₅₂H₅₂₃N₈₃O₁₀₉ | m/7: 1095.9 (avg) | M111 |
| 227 | C₃₅₁H₅₂₃N₈₃O₁₀₈ | m/7: 1091.8 (avg) | M111 |
| 228 | C₃₅₄H₅₂₇N₈₃O₁₀₉ | m/7: 1100.3 (avg) | M111 |
| 229 | C₃₅₈H₅₃₁N₈₃O₁₁₀ | m/7: 1109.3 (avg) | M111 |
| 230 | C₃₅₈H₅₃₂N₈₄O₁₀₉ | m/7: 1109.3 (avg) | M111 |
| 231 | C₃₅₄H₅₂₆N₈₂O₁₁₁ | m/7: 1101.9 (avg) | M111 |
| 232 | C₃₅₂H₅₂₃N₈₁O₁₁₀ | m/7: 1094.0 (avg) | M111 |
| 233 | C₃₅₃H₅₂₅N₈₁O₁₁₀ | m/7: 1096.4 (avg) | M111 |
| 234 | C₃₅₈H₅₂₈N₈₂O₁₁₀ | m/7: 1107.2 (avg) | M111 |
| 235 | C₃₅₅H₅₂₉N₈₃O₁₁₀ | m/7: 1104.0 (avg) | M111 |
| 236 | C₃₅₅H₅₂₈N₈₄O₁₁₀ | m/7: 1106.0 (avg) | M111 |
| 237 | C₃₅₃H₅₂₆N₈₂O₁₀₉ | m/8: 959.5 (avg) | M111 |
| 238 | C₃₅₈H₅₂₈N₈₂O₁₁₀ | m/7: 1107.2 (avg) | M111 |
| 239 | C₃₅₈H₅₃₁N₈₅O₁₀₉ | m/7: 1111.2 (avg) | M111 |
| 240 | C₃₅₇H₅₂₉N₈₅O₁₀₉ | m/7: 1109.1 (avg) | M111 |
| 241 | C₃₅₇H₅₂₉N₈₅O₁₀₉ | m/7: 1109.1 (avg) | M111 |
| 242 | C₃₅₇H₅₂₉N₈₅O₁₀₉ | m/7: 1109.0 (avg) | M111 |
| 243 | C₃₅₆H₅₂₇N₈₅O₁₀₉ | m/7: 1107.5 (avg) | M111 |
| 244 | C₃₅₈H₅₃₂N₈₂O₁₁₀ | m/7: 1107.6 (avg) | M111 |
| 245 | C₃₅₈H₅₃₃N₈₃O₁₀₉ | m/7: 1107.5 (avg) | M111 |
| 246 | C₃₄₈H₅₁₂N₈₄O₁₁₀S₂ | m/5: 1539.578 (mono) | M64 |
| 247 | C₃₄₈H₅₁₂N₈₄O₁₁₀S₂ | m/5: 1539.578 (mono) | M64 |
| 248 | C₃₄₇H₅₁₅N₈₃O₁₀₅S₂ | m/5: 1518.781 (mono) | M64 |
| 249 | C₃₄₉H₅₁₇N₈₃O₁₀₇S₂ | m/5: 1530.586 (mono) | M64 |
| 250 | C₃₄₈H₅₁₄N₈₄O₁₀₈S₂ | m/5: 1533.58 (mono) | M64 |
| 251 | C₃₅₁H₅₁₉N₈₃O₁₀₉S₂ | m/5: 1542.014 (mono) | M64 |
| 252 | C₃₄₉H₅₁₇N₈₁O₁₀₉S₂ | m/5: 1531.2 (mono) | M64 |

(avg) means average molecular mass peak, (mono) means monoisotopic peak in the MS spectrum.

### Example 1: Design of Compounds and Binding Motifs (BM)

The hFGF23 antagonistic polypeptides as disclosed herein are designed molecules developed on a three-helix bundle protein domain derived from the Z domain of staphylococcal protein A, that bind to the Fc part of immunoglobulins. By randomizing selected amino acids in the range residues 9 to residue 35 on the two first helices, which constitute the binding surface to Fc, large libraries were constructed, from which potent hFGF23 binders were isolated by a variety of methods. The randomization of the Fc binding surface residues in the range residues 9 to residue 35 and subsequent selection of polypeptide variants lead to replacement of the Fc binding capacity with a capacity for binding hFGF23 (selection #1). The binding motif (BM) thus corresponds to residues 9 to 35. A new library was designed after analysis of the results from selection #1 by randomizing other residues within the binding motif (BM) to improve binding capacity for hFGF23 even further (selection #2). The polypeptide variants from the selection #2 were subsequently optimized for potency by amino acid substitutions within the binding motif (BM) and outside the binding motif (BM) (selection #3) leading to the hFGF23 antagonistic polypeptides as disclosed herein. Polypeptide variants from selection #1 and selection #2 were optimized for in vivo half-life by identifying positions in the polypeptide backbone, where the protraction moiety could be attached and retain binding capacity for hFGF23.

The hFGF23 antagonistic polypeptides of the invention can be functionalized with genetic fusions to protein modules of interest or by covalent amino acid residue side chain attachment to functional moieties, such as toxins, imaging agents, and/or one or more protraction moieties.

### Example 2: Inhibitory Effect on hFGF23-Induced Signalling in Serum-Response-Luciferase Reporter Gene Assay

The purpose of this assay is to demonstrate and characterize to what degree the hFGF23 antagonistic polypeptide derivatives (“polypeptides”) provide inhibitory effect on the hFGF23-induced signalling mediated by both FGFR1c as receptor and alpha-klotho (KL) as co-receptor. hFGF23 is known to signal specifically through this trimeric complex of FGFR1c/KL/hFGF23 via the MAPK/ERK pathway. Treatment of cells with hFGF23 leads to luciferase expression and after addition of detection reagent eventually to a luminescence signal. Compounds of the invention inhibited the hFGF23-induced signalling with an IC₅₀<1 nM or reduced the hFGF23-induced signalling with >50% at 1 nM.

### Assay Principle

A reporter gene assay was established to determine the ability of the polypeptide compounds to inhibit signalling. We used luciferase under control of the Serum-Response-Element (SRE) to measure canonical hFGF23-induced signalling.

HEK293 cells were first stably transfected with a human KL expression plasmid encoding zeocin resistance gene for mammalian selection. After generating a monoclonal HEK293/KL cell line from a single clone in the presence of 50 μg/ml Zeocin (Invitrogen #R25001), the reporter plasmid based on pGL4.33[luc2P/SRE/Hygro](Promega #E1340) encoding luciferase under the control of the SRE was stably transfected. The result was a HEK293/KL/SRE cell line derived from a single clone named 17-3 under 200 μg/ml hygromycin selection. HEK293/KL/SRE cells were maintained under standard cell culture conditions at 37° C. in full media based on DMEM (Gibco #31966-021) supplemented with 1% Penicillin-Streptomycin (Gibco #15140-122) and 10% FBS (Thermo Fisher Scientific #10091148), 50 μg/ml Zeocin and 200 μg/ml Hygromycin (Invitrogen #10687010).

In order to assess polypeptide compound-mediated inhibition of hFGF23-induced signalling, HEK293/KL/SRE cells were seeded in starvation media in poly-D-lysine coated black 384-well plates (Corning #354663) with a density of 10.000 cells per well. Starvation media was based on DMEM supplemented with 0.5% human serum albumin and 1% Penicillin-Streptomycin. The next day, polypeptide compounds and recombinant hFGF23 (SEQ ID NO:5) (RnD Systems #2604-FG-025) were diluted in starvation media and co-incubated for 1 h at 22° C. prior to addition to cells. Cells were incubated for 4 h at 37° C. followed by lysis and detection of luminescence using EnVision 2104 μlate reader with ultrasensitive luminescence detection (PerkinElmer).

Polypeptide compounds were tested either in single-dose or dose-response. For single-dose experiments, polypeptide compounds were applied at a final concentration of 1 nM. For dose-response experiments, polypeptide compounds were serial-diluted and applied to the cells at concentrations ranging between 50 and 0.032 nM. For both assay setups a concentration of 45 μM hFGF23 was applied corresponding to hFGF23's EC₈₀. In single-dose experiments, compound response data were normalized to the minimum and maximum response in the absence and presence of hFGF23, respectively. Results were expressed as “% residual activity”. The following formula was applied:

$$residual activity in \% = \frac{y - minimum}{maximum - minimum} * 100 \%$$

y=luminescence response of polypeptide compound
minimum=no hFGF23 treatment
maximum=hFGF23 treatment

Dose-response experiments serve the purpose of determining the half-maximal inhibitory concentration (IC₅₀) in order to compare the in vitro potency of the compounds. For that, a four-parameter logistic equation (4PL-fit) was applied:

$$Y = bottom + \frac{top - bottom}{1 + {( \frac{IC 50}{X} )}^{Hillslope}}$$

Y=response, decreasing as x is increasing
x=concentration of polypeptide compound
IC₅₀=half-maximal inhibitory concentration
HillSlope=steepness of linear part of dose-response curve

Analysis for both single-dose and dose-response experiments was done in Datalab using Python scripts.

### Materials:

Zeocin (Invitrogen #R2500)
Hygromycin (Invitrogen #10687010)
pGL4.33[luc2P/SRE/Hygro](Promega #E1340)
DMEM (Gibco #31966-021)
Penicillin-Streptomycin (Gibco #15140-122)
FBS (Thermo Fischer #10091148)
Hygromycin (Invitrogen #10687010)
Poly-D-lysine coated black 384-well plates (Corning #354663)
Recombinant human FGF23 (amino acids 25-251) (RnD Systems #2604-FG-025)

### Results

The results are shown in Table 6 which summarises inhibitory potencies in the form of IC₅₀ values of the compounds of the invention or the residual hFGF23 activity at a compound concentration of 1 nM. An IC₅₀ value of less than 1 nM or a % residual activity at 1 nM below 50% implies that a compound has surprisingly strong inhibitory activity under the experimental conditions.

TABLE 6
hFGF23-inhibitory potencies (IC₅₀) and % residual activity at 1 nM

| | | % residual | | | % residual |
|---|---|---|---|---|---|
| | IC₅₀ | activity at 1 | | IC₅₀ | activity at 1 |
| Chem. | (nM) | nM | Chem. | (nM) | nM |
| 4 | 0.803 | | 129 | 0.286 | |
| 5 | 0.646 | | 130 | 0.262 | |
| 6 | 0.893 | | 131 | 0.413 | |
| 7 | 0.497 | | 132 | 0.432 | |
| 8 | 0.722 | | 133 | 0.344 | |
| 9 | | 21 | 134 | 0.412 | |
| 10 | | 38 | 135 | 0.172 | |
| 11 | | 46 | 136 | 0.300 | |
| 12 | | 38 | 137 | 0.283 | |
| 13 | | 41 | 138 | 0.215 | |
| 14 | | 46 | 139 | 0.290 | |
| 15 | | 38 | 140 | 0.112 | |
| 16 | | 46 | 141 | 0.237 | |
| 17 | | 49 | 142 | 0.142 | |
| 18 | | 44 | 143 | 0.181 | |
| 19 | | 47 | 144 | 0.163 | |
| 20 | | 47 | 145 | 0.591 | |
| 21 | | 47 | 146 | 0.428 | |
| 22 | | 30 | 147 | 0.917 | |
| 23 | | 33 | 148 | 0.407 | |
| 24 | | 48 | 149 | | 29 |
| 25 | | 26 | 150 | 0.514 | |
| 26 | | 42 | 151 | 0.465 | |
| 27 | | 32 | 152 | 0.391 | |
| 28 | | 39 | 153 | 0.204 | |
| 29 | | 30 | 154 | 0.372 | |
| 30 | | 44 | 155 | 0.362 | |
| 31 | | 42 | 156 | 0.298 | |
| 32 | | 48 | 157 | 0.614 | |
| 33 | | 38 | 158 | 0.363 | |
| 34 | | 37 | 159 | 0.546 | |
| 35 | | 39 | 160 | 0.591 | |
| 36 | | 49 | 161 | | 25 |
| 37 | | 40 | 162 | | 13 |
| 38 | | 42 | 163 | | 11 |
| 39 | | 34 | 164 | | 17 |
| 40 | | 42 | 165 | 0.239 | |
| 41 | | 42 | 166 | 0.322 | |
| 42 | | 44 | 167 | 0.860 | |
| 43 | | 42 | 168 | 0.550 | |
| 44 | | 32 | 169 | | 39 |
| 45 | | 27 | 170 | | 23 |
| 46 | | 41 | 171 | | 37 |
| 47 | | 46 | 172 | 0.221 | |
| 48 | | 47 | 173 | 0.223 | |
| 49 | | 47 | 174 | 0.325 | |
| 50 | | 31 | 175 | 0.093 | |
| 51 | | 30 | 176 | 0.146 | |
| 52 | | 43 | 177 | | 9 |
| 53 | | 41 | 178 | | 11 |
| 54 | | 41 | 179 | 0.259 | |
| 55 | | 43 | 180 | 0.238 | |
| 56 | | 49 | 181 | 0.198 | |
| 57 | | 45 | 182 | 0.456 | |
| 58 | | 41 | 183 | | 20 |
| 59 | | 44 | 184 | | 34 |
| 60 | | 29 | 185 | | 15 |
| 61 | | 44 | 186 | 0.134 | |
| 62 | | 41 | 187 | 0.231 | |
| 63 | | 40 | 188 | | 21 |
| 64 | | 37 | 189 | | 12 |
| 65 | | 37 | 190 | 0.334 | |
| 66 | | 49 | 191 | | 14 |
| 67 | | 48 | 192 | 0.264 | |
| 68 | | 38 | 193 | 0.503 | |
| 69 | | 45 | 194 | 0.140 | |
| 70 | | 40 | 195 | 0.206 | |
| 71 | | 27 | 196 | 0.204 | |
| 72 | | 30 | 197 | 0.055 | |
| 73 | | 29 | 198 | 0.159 | |
| 74 | | 37 | 199 | 0.105 | |
| 75 | | 35 | 200 | 0.081 | |
| 76 | | 34 | 201 | 0.106 | |
| 77 | | 22 | 202 | 0.742 | |
| 78 | | 24 | 203 | 0.077 | |
| 79 | | 27 | 204 | 0.083 | |
| 80 | | 20 | 205 | 0.087 | |
| 81 | | 34 | 206 | 0.076 | |
| 82 | | 27 | 207 | 0.074 | |
| 83 | | 24 | 208 | 0.109 | |
| 84 | | 29 | 209 | 0.050 | |
| 85 | | 28 | 210 | 0.039 | |
| 86 | | 27 | 211 | 0.137 | |
| 87 | | 36 | 212 | 0.093 | |
| 88 | | 18 | 213 | 0.035 | |
| 89 | 0.084 | | 214 | 0.034 | |
| 90 | | 21 | 215 | 0.196 | |
| 91 | | 25 | 216 | 0.641 | |
| 92 | | 20 | 217 | 0.657 | |
| 93 | | 17 | 218 | 0.583 | |
| 94 | | 24 | 219 | 0.379 | |
| 95 | | 25 | 220 | 0.101 | |
| 96 | | 41 | 221 | 0.195 | |
| 97 | | 40 | 222 | 0.258 | |
| 98 | | 47 | 223 | 0.340 | |
| 99 | | 48 | 224 | 0.273 | |
| 100 | | 39 | 225 | 0.293 | |
| 101 | | 49 | 226 | 0.216 | |
| 102 | | 41 | 227 | 0.483 | |
| 103 | | 47 | 228 | 0.305 | |
| 104 | 0.527 | | 229 | 0.226 | |
| 105 | 0.343 | | 230 | 0.307 | |
| 106 | 0.083 | | 231 | 0.104 | |
| 107 | 0.127 | | 232 | 0.149 | |
| 108 | 0.166 | | 233 | 0.166 | |
| 109 | 0.391 | | 234 | 0.473 | |
| 110 | 0.258 | | 235 | 0.218 | |
| 111 | 0.332 | | 236 | 0.273 | |
| 112 | 0.681 | | 237 | 0.200 | |
| 113 | 0.722 | | 238 | 0.558 | |
| 114 | 0.329 | | 239 | 0.158 | |
| 115 | 0.320 | | 240 | 0.182 | |
| 116 | 0.556 | | 241 | 0.185 | |
| 117 | 0.413 | | 242 | 0.372 | |
| 118 | 0.804 | | 243 | 0.100 | |
| 119 | 0.625 | | 244 | 0.274 | |
| 120 | 0.328 | | 245 | 0.467 | |
| 121 | 0.263 | | 246 | 0.171 | |
| 122 | 0.212 | | 247 | 0.051 | |
| 123 | 0.307 | | 248 | 0.882 | |
| 124 | 0.176 | | 249 | 0.511 | |
| 125 | 0.212 | | 250 | 0.158 | |
| 126 | 0.261 | | 251 | 0.172 | |
| 127 | 0.352 | | 252 | 0.083 | |
| 128 | 0.169 | | 3 | 0.296 | |

Analysis of the data shows that the compounds as disclosed herein are capable of inhibiting hFGF23-induced signalling. It is demonstrated that the compounds—having high % sequence identity, such as at least 93% sequence identity—retain inhibitory activity towards hFGF23. The compounds are thus suitable for use in the treatment of hypophosphatemic disorders, such as X-linked hypophosphatemic rickets (XLH).

### Example 3: Surface Plasmon Resonance (SPR) Characterization of the Interaction Between hFGF23 Antagonistic Polypeptides and hFGF23

The purpose of this assay is to demonstrate that hFGF23 antagonistic polypeptides (“polypeptides”) of the invention bind to hFGF23 and to determine the binding affinity. Binding studies are executed on a Biacore™ T200, Biacore™ 8K, or Biacore™ 8K+ (all from Cytiva), instruments designed to quantify molecular interactions in real-time utilizing Surface Plasmon Resonance (SPR). The experiments are conducted at either 10° C. or 25° C., while samples are maintained at 10° C. in the sample compartment. The SPR instruments' reported signals, expressed in response units (RU), directly correlate with the mass on the sensor chip surfaces within eight parallel channels, each containing two serial flow cells.

The procedure begins with the covalent immobilization of an in-house anti-HPC4 monoclonal antibody onto both flow cells of a Series S Sensor Chip CM4 (Cytiva) using the Amine Coupling Kit (Cytiva) following the manufacturer's guidelines. Subsequently, HPC4-tagged recombinant hFGF23 (SEQ ID NO:6) is captured by injecting it over flow cell 2. This creates an active target surface on flow cell 2 and a reference surface in flow cell 1, featuring only immobilized anti-HPC4 antibody.

The binding of the polypeptides to the captured target is examined by injecting the analyte over both flow cells, enabling comparative analyses of different compounds' binding to the captured target relative to the reference surface. Compounds of the invention are serially diluted into the running buffer (10 mM HEPES pH 7.4, 150 mM NaCl, 3 mM EDTA, 0.05% v/v Surfactant P20), injected at a rate of 50 μl min⁻¹ for 240 seconds, and allowed to dissociate for 900 seconds. After each analyte injection cycle, the CM4 surface is regenerated via 10 mM Glycine pH 1.5 injections. This step eliminates the captured target and any bound polypeptides from the immobilized antibody surface, preparing it for the subsequent interaction sample pair. The regeneration procedure does not remove the covalently immobilized anti-HPC4 capture antibody from the chip surface.

Polypeptide binding curves undergo processing, which involves subtraction of reference surface signals as well as blank buffer injections over captured hFGF23. This process allows for the correction of instrument noise, bulk shift, and drift during sample injections. The polypeptides association and dissociation rate constants, namely kₐ (association rate) and k_{d} (dissociation rate), are extracted by globally fitting a 1:1 Langmuir model to the data using Biacore Insight Evaluation Software (version 5.0.18.22102). The affinity between compounds of the invention and the target is quantified by the equilibrium dissociation constant (K_{D}), calculated from the determined kinetics by the equation K_{D}=k_{d}/kₐ. The results are presented in Table 7 below.

TABLE 7
Affinity values

| Chem. | K_{D} (nM) |
|---|---|
| 89 | 1.61 |
| 175 | 0.99 |
| 197 | 1.05 |
| 201 | 1.67 |
| 204 | 0.81 |
| 205 | 0.86 |
| 206 | 1.80 |

Analysis of the data shows that the compounds exhibit desirable binding towards hFGF23. It was further demonstrated that compounds having high % sequence identity bind to hFGF23 with an affinity (K_{D}) of 2 nM or less, and in particular 1.8 nM or less.

### Example 4: Surface Plasmon Resonance (SPR) Characterization of the Interaction Between hFGF23 Antagonistic Polypeptides and FGF23 from Non-Human Species

The purpose of this assay is to demonstrate that hFGF23 antagonistic polypeptides (“polypeptides”) of the invention bind to FGF23 from non-human species and to determine the binding affinity. The assay was conducted similarly to Example 3, except hFGF23 was replaced with FGF23 from dog, pig or monkey. The results are presented in Table 8 below.

TABLE 8
Affinity values

| Chem. | K_{D} (nM, dog) | K_{D} (nM, pig) | K_{D} (nM, monkey) |
|---|---|---|---|
| 89 | 7.63 | 1.10 | 3.12 |
| 175 | 4.12 | 0.66 | 1.65 |
| 197 | 4.32 | 0.84 | 1.84 |
| 201 | 6.24 | 0.61 | 2.61 |
| 204 | 2.92 | 0.56 | 0.39 |
| 205 | 3.32 | 0.56 | 1.33 |

Analysis of the data shows that the compounds exhibit desirable cross-species reactivity binding towards FGF23 from dog, pig and monkey.

### Example 5: Pharmacokinetic Study 1 in Minipigs

The aim of the study was to determine the half-life in blood following i.v. administration of certain compounds of the invention.

The studies were done in female Göttingen minipigs (Ellegaard Minipigs A/S, Dalmose, Denmark) with a body weight of approximately 25 kg. The polypeptides were administered to the minipigs intravenously, n=3 per group, and the dose was 10 nmol/kg. The vehicle of the formulations was 50 mM phosphate and 70 mM sodium chloride at pH 7.4.

Blood was sampled for up to 312 hours through an indwelling permanent venous catheter and collected in EDTA tubes (Sarstedt, Germany). Plasma was separated and analysed for compound concentration using Liquid chromatography mass spectrometry (LC-MS).

Non-compartmental (NCA) pharmacokinetic analysis was done in PHOENIX® WINNONLIN® v. 8.1 (Certara L. P. Princeton, NJ, USA). The terminal elimination phase was fitted via linear regression with uniform weighting. Nominal sampling times and actual doses were used for NCA.

Concentrations of polypeptides in minipig plasma were assessed with plasma protein precipitation followed by turboflow liquid chromatography mass spectrometry (TF-LC-MS).

Calibration standards and quality control samples were prepared by spiking compounds into blank plasma followed by serial dilution. All plasma samples underwent sample preparation by addition of four volumes of ethanol to one volume of plasma sample. The precipitation solvent contained 20 nM internal standard. Following centrifugation at 4° C., the supernatant was diluted with one volume of purified water containing 1% formic acid. Precipitated plasma samples were first cleaned up and concentrated on a TurboFlow Cyclone column (0.5×50 mm from Thermo Fisher Scientific) operated at 30° C. and then transferred to an analytical column. The analytical column was an XBridge Peptide BEH C18 column (2.1×50 mm, 3.5 μm particle size from Waters) and operated at 60° C. Analytical separations were conducted with a linear gradient. Mobile phase A was water with 5% organic solvent (acetonitrile/methanol (1:1, by volume)) and 1% formic acid, and mobile phase B was water with 95% organic solvent (acetonitrile/methanol (1:1, by volume)) and 1% formic acid. The mass spectrometer coupled to the TF-LC system was either a TSQ Altis or a Q Exactive (both from Thermo Fisher Scientific). The mass spectrometers were operated in positive ionization mode and selected reaction monitoring on the TSQ Altis and parallel reaction monitoring on the Q Exactive.

Samples were quantified using linear regression against the response from the calibration standards with 1/X² weighting. The response was the analyte peak area divided by the internal standard peak area. Measured concentration for quality control samples and calibration standards were within ±15% of the nominal concentration, except for one quality control sample that was −17% of nominal concentration. The lower limit of quantification was between 0.250 nM and 1.00 nM.

### Results

Results are shown in Table 9 below, wherein column T1/2 shows the half-life (T1/2) in blood. A high half-life value is desirable.

TABLE 9
Pharmacokinetic parameters after intravenous administration

| Chem. | T½ (h) | Protractor |
|---|---|---|
| 197 | 47.1 | C20 diacid |
| 111 | 47.9 | C18 diacid |
| 107 | 49.6 | C18 diacid |
| 89 | 51.6 | C18 diacid |
| 204 | 51.9 | C20 diacid |
| 201 | 58.8 | C20 diacid |
| 106 | 59.7 | C20 diacid |

No adverse clinical observations deemed related to the test compounds were observed during the study.

The results show that protraction at position 58 with the C18 diacid or C20 diacid results in clinically meaningful half-life in blood, in polypeptide compounds having high sequence identity.

A long half-life indicates that the given compound is capable of providing its inhibitory effect for a long period of time after dosing in comparison with a compound having a relatively shorter half-life. Longer half-life permits less frequent dosing when in use, which is desirable to provide an increased convenience for the patients.

### Example 6: Pharmacokinetic and Pharmacodynamic Study in Dogs

The aim of the present study was to evaluate the pharmacokinetics and pharmacodynamics of compounds of the invention in male Beagle dog following single subcutaneous (s.c.) injection. A single s.c. injection of Chem. 206, Chem. 207 and Chem. 208 was administrated to male Beagle dog (n=3 per compound, target dose 10 nmol/kg). The composition of injection formulation is listed as follow: 20 mM L-histidine; 150 mM L-arginine hydrochloride; pH=6.49-6.51.

Fifteen male beagle dogs from Marshall, Beijing, China, aged between 2.60 and 9.10 years old and weighing between 11.3 and 15.0 kg, were included in the study. The dogs were group-housed during the study, and each animal was tattooed with unique number for identification. The housing rooms were maintained at a temperature of 16-25° C. and a humidity level of 40-70%. The light cycle was set to 12 hours of light and 12 hours of dark. During study period, dogs were fed once daily of commercially available canine food (Canine Adult Medium Dogs Dry Dog Food, ROYAL CANIN®, Shanghai, China; 150-225 g/dog/day) at 13:00. Animals were overnight fasted for the morning sampling. Animals had free access to quality drinking water. On the day of experiment initiation, dogs received a s.c. single bolus injection of test compounds around 9:00. Blood samples (1.2 mL at each time point) were collected in EDTA-coated tubes at the following time points for the measurement of plasma concentrations of test compounds and plasma phosphate: pre-dose (0 min), 5, 15, 30, 45 min, 1, 1.5, 2, 4, 7, 10 h, 24, 30, 48, 72, 96, 120, 144, 168, 192, 216, 240, 264, 288, 312, 336 h. Blood samples were kept on ice until centrifugation (4 min, 4° C., 4000 rpm). After centrifugation, two sets of 100 μl EDTA plasma (one for quantification of test compound and one for quantification of phosphate) at each sampling time point were transferred immediately into Micronic tubes and stored at −20° C. until analyses.

### Plasma Analysis of Test Compound

Plasma concentrations of Chem. 206, Chem. 207 and Chem. 208 were assayed by plasma protein precipitation and analysed by turboflow liquid chromatography mass spectrometry (TF-LC-MS). Calibrators were prepared by spiking blank dog plasma with Chem. 206, Chem. 207 and Chem. 208 in the range from 0.5 to 2000 nM. Calibrators, plasma blanks or study samples were prepared for TF-LC-MS by protein precipitation by adding three volumes of ethanol containing 50 nM of internal standard (ISTD) (Chem. 175) to one volume of sample followed by centrifugation at 4000 rpm at 4° C. for 60 minutes. One volume of supernatant was mixed with two volumes of water (with 0.1% formic acid). The mixture was analysed by TF-LC-MS using a Cyclone turboflow column (0.5×50 mm, ThermoFisher Scientific) and an Agilent poroshell 120 SB-C18 column (2.1×50 mm, 2.7 μm particle). A gradient elution was conducted using mobile phase A (consisting of milli-Q water with 0.1% formic acid and 5% methanol/acetonitrile (50/50)) and mobile phase B (consisting of methanol/acetonitrile (50/50) with 0.1% formic acid and 5% milli-Q water). The mass spectrometer was operated in positive ionization mode. An Orbitrap Exploris 240 mass spectrometer (ThermoFisher Scientific) was used as detector in parallel reaction monitoring mode (Chem. 206: m/z 1912.4600@1748.5375; Chem. 207: m/z 1926.9600@1767.5369; Chem. 208: m/z 1905.4500@2211.0012; Chem. 175: m/z 1923.4500@1762.8643). Linear calibration curves (weighed 1/x2) were used for calculating the concentration in the plasma samples. Quality control samples were included. The deviation between nominal and calculated concentration in the calibrators and quality control samples were below 15%. The lower limit of quantification (LLOQ) is 2.0 nM.

### Pharmacokinetic Analysis of Test Compound

Plasma concentration values of test compounds at each sampling time point were imported into PHOENIX® WINNONLIN® version 8.4.0.6172 for non-compartmental analysis (NCA) using Linear Up Log Down method and weighting 1/(Y*Y). Actual sampling time was used for NCA calculations. Individual concentration values below LLOQ were treated as zero for the timepoints before the first detectable exposure and were treated as missing values for the timepoints after Tmax. Actual doses, calculated using the actual concentration of the formulation, were used for NCA analysis.

Results are shown in Table 10 below.

TABLE 10
Pharmacokinetic parameters after s.c. administration

| Chem. | T½ (h) | Protractor |
|---|---|---|
| 206 | 77 | C20 diacid |
| 207 | 74 | C20 diacid |
| 208 | 88 | C20 diacid |

At the test dose, compounds were well-tolerated, and no adverse effects were observed in the test animals. The results show that protraction at position 58 with C20 diacid results in clinically meaningful half-life in blood, in polypeptide compounds having high sequence identity.

### Example 7: Plasma Phosphate Levels in Minipigs and Dogs

In order to determine the in vivo effect of the compounds of the invention, plasma phosphate levels were measured in the PK studies described in Example 5 and Example 6.

### Method Serum Pi:

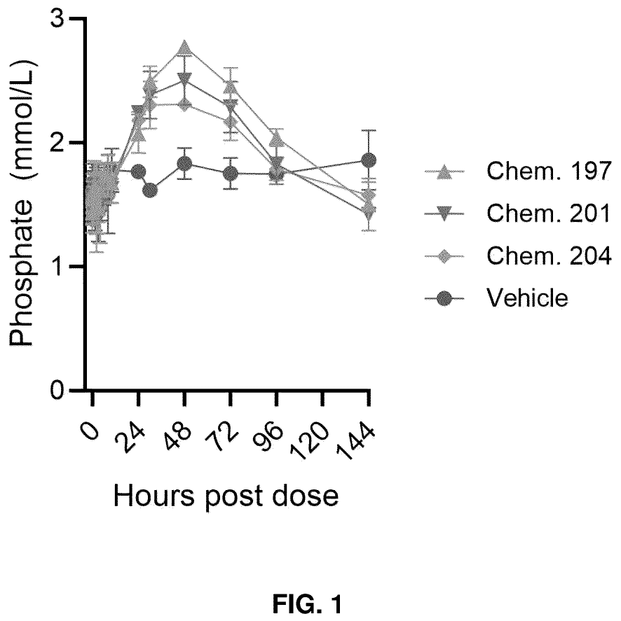

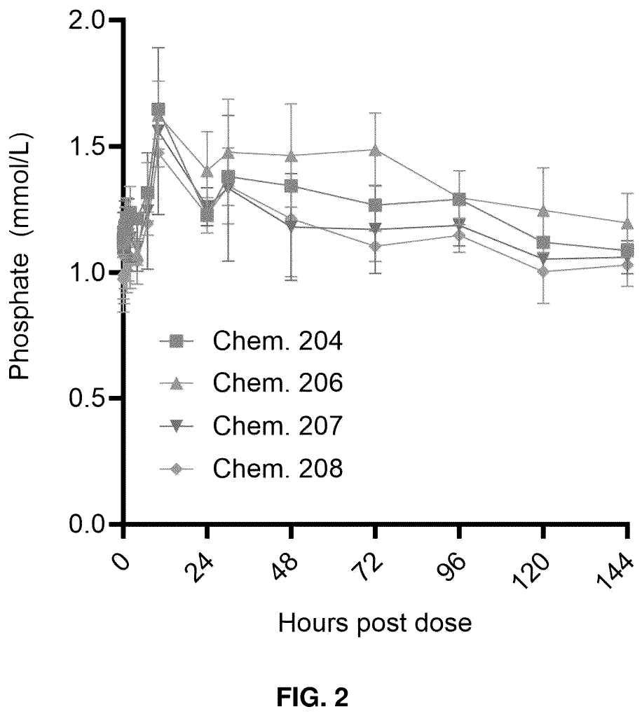

Plasma phosphate was measured with a PHOS2 inorganic phosphate assay v2.0 (Roche) on a Cobas pro c503 analytical instrument. Animals were administered a single intravenous dose of 10 nmol/kg (minipigs, Example 5) or a single subcutaneous dose of 10 nmol/kg (Beagle dogs, Example 6) of compound. Plasma samples for analysis were collected at dosing (0), at 5, 15 and 30 minutes, and at 1, 1.5, 2, 3, 4, 6, 8, 10, 24, 30, 48, 72, 96 and 144 hours post dose. Data is reported in FIG. 1/Table 11 and FIG. 2/Table 12 showing plasma phosphate levels in mmol/L.

TABLE 11
Plasma phosphate levels (mmol/L) in
minipigs after intravenous dosing

| Time | | Chem. 197 | Chem. 201 | Chem. 204 | Vehicle |
|---|---|---|---|---|---|
| 0 | min | 1.44 | 1.45 | 1.44 | 1.59 |
| 5 | min | 1.44 | 1.56 | 1.43 | 1.55 |
| 15 | min | 1.57 | 1.66 | 1.61 | 1.39 |
| 30 | min | 1.59 | 1.57 | 1.59 | 1.52 |
| 1 | h | 1.63 | 1.60 | 1.71 | 1.67 |
| 1.5 | h | 1.64 | 1.66 | 1.66 | 1.50 |
| 2 | h | 1.50 | 1.48 | 1.48 | 1.53 |
| 3 | h | 1.47 | 1.39 | 1.42 | 1.51 |
| 4 | h | 1.62 | 1.49 | 1.48 | 1.48 |
| 6 | h | 1.73 | 1.65 | 1.66 | 1.62 |
| 8 | h | 1.73 | 1.62 | 1.70 | 1.59 |
| 10 | h | 1.68 | 1.63 | 1.71 | 1.78 |
| 24 | h | 2.07 | 2.24 | 2.18 | 1.77 |
| 30 | h | 2.49 | 2.39 | 2.31 | 1.62 |
| 48 | h | 2.78 | 2.50 | 2.31 | 1.83 |
| 72 | h | 2.46 | 2.29 | 2.17 | 1.75 |
| 96 | h | 2.04 | 1.83 | 1.79 | 1.75 |
| 144 | h | 1.50 | 1.42 | 1.58 | 1.86 |

TABLE 12
Plasma phosphate levels (mmol/L) in dogs after s.c. administration

| Time | | Chem. 204 | Chem. 206 | Chem. 207 | Chem. 208 |
|---|---|---|---|---|---|
| 0 | min | 1.13 | 1.12 | 1.16 | 0.97 |
| 5 | min | 1.12 | 1.12 | 1.13 | 0.99 |
| 15 | min | 1.16 | 1.14 | 1.16 | 0.99 |
| 30 | min | 1.18 | 1.16 | 1.17 | 1.06 |
| 1 | h | 1.19 | 1.20 | 1.16 | 1.06 |
| 1.5 | h | 1.22 | 1.22 | 1.15 | 1.01 |
| 2 | h | 1.22 | 1.18 | 1.17 | 1.07 |
| 3 | h | 1.24 | 1.23 | 1.14 | 1.04 |
| 4 | h | 1.21 | 1.07 | 1.10 | 1.04 |
| 6 | h | 1.32 | 1.32 | 1.24 | 1.19 |
| 8 | h | 1.65 | 1.62 | 1.56 | 1.47 |
| 10 | h | 1.23 | 1.40 | 1.26 | 1.23 |
| 24 | h | 1.38 | 1.48 | 1.33 | 1.34 |
| 30 | h | 1.34 | 1.46 | 1.18 | 1.21 |
| 48 | h | 1.27 | 1.49 | 1.17 | 1.10 |
| 72 | h | 1.29 | 1.30 | 1.19 | 1.15 |
| 96 | h | 1.12 | 1.25 | 1.05 | 1.00 |
| 144 | h | 1.09 | 1.20 | 1.06 | 1.03 |

The results show that compounds of the invention are capable of increasing plasma phosphate levels in vivo.

### Example 8: Crystallization and Epitope Mapping of Chem. 3 Using X-Ray Crystallography

In order to determine where compounds of the invention bind to hFGF23, crystallization studies were performed to determine epitope residues on hFGF23.

hFGF23 and Test Compound for Complex Purification and Initial Crystallization

The complex consisting of the hFGF23 core domain (SEQ ID NO:4) and Chem. 3 (SEQ ID NO:10) was prepared by mixing 1.5× molar excess of Chem. 3 (SEQ ID NO: 10) with hFGF23 core domain (SEQ ID NO:4), incubating for 2 hours at 4° C. and purifying by size exclusion chromatography (SEC) using a Superdex75 16/60 column running in 10 mM HEPES pH 7.5, 500 mM NaCl. The purified complex was collected from the SEC peak fractions, concentrated to 3.7-7.4 mg/ml and used for crystallization trials in 96 well sitting drops using commercial crystallization screens incubated at 18° C. Initial crystals were obtained in a drop containing 0.18 μl reservoir solution (0.2 M Potassium formate, 20% (w/v) PEG 3350) and 0.36 μl protein solution. The initial crystals were used to prepare a seedstock for subsequent seeding crystallization trails. The seedstock was prepared as follows: 1) crushing the initial crystals in the drop, 2) transfer the drop containing the crushed crystals into a seed bead tube (HR2-320 Hampton research) containing 50 μl reservoir solution (0.2 M Potassium formate, 20% (w/v) PEG 3350) and 3) shaking on a vortex mixer for 2 min.

Crystallization and Structure Determination of hFGF23 Core Domain (SEQ ID NO:4) in Complex with Chem. 3

Crystals were obtained at 18° C. by seeding 35 nl seedstock into a drop containing 0.36 μl protein solution and 0.36 μl reservoir solution (0.2 M Calcium acetate hydrate, 20% w/v Polyethylene glycol 3,350). The crystals were cryo-protected in glycerol and frozen in liquid nitrogen. Synchrotron X-ray diffraction data was collected at the Swiss Light Source, processed in XDS/XSCALE to a resolution of 1.8 Å in space group 4 (P21). The structure was determined by molecular replacement running in Phenix using the following pdb entries as search models; 2P39 and 5DJT (chain B only). The solution from molecular replacement was in space group 4 (P21) and had two complexes in the asymmetric unit (complex 1 and complex 2). The structure was refined in Phenix to 1.8 Å resolution.

### Brief Description and Interpretation of the Structure

The two complexes in the asymmetric unit showed high structural similarity with RMSD of 0.658 Å (backbone) and 1.058 Å (all atom) calculated in PyMOL using “Align” without outlier rejection. The overall structure of the hFGF23 core domain (SEQ ID NO:4) resembled the previous (pdb entry 2P39) crystal structure of hFGF23 with backbone RMSD of 1.304 Å calculated in PyMOL using “Align” without outlier rejection.

The interaction between hFGF23 and its receptor (heterodimer of FGFR1c and alpha-klotho) has been characterized previously by x-ray crystallography (pdb entry 5W21). A structural comparison of the hFGF23/receptor complex (5W21) and the Chem. 3/hFGF23 core domain (SEQ ID NO:4) complex showed a significant overlap between the binding sites of the hFGF23 receptor and Chem. 3 on hFGF23, confirming that Chem. 3 directly prevents hFGF23 from interaction with the hFGF23 receptor and thereby inhibits the function of hFGF23.

### Determination of the Epitope of Chem. 3

Epitope residues (3.5 Å distance between any non-hydrogen atoms in Chem. 3 and hFGF23) comprises the following residues in hFGF23 (SEQ ID NO:1): M74, R76, T86, R91, F108, P110, R114, L166, I167 and N170.

### Example 9: In Silico Epitope Determination

Alphafold v. 2.3 (www.biorxiv.org/content/10.1101/2021.10.04.463034v2) was used to in silico fold hFGF23 (SEQ NO:1) with Chem. 206, Chem. 207, and Chem. 208, respectively. For multiple sequence alignment, the reduced database setting was applied and the number of recycles was set to 5. All other model parameters were set to ‘default’.

The identified epitopes determined at 3.5 Å distance between any non-hydrogen atoms in the tested compound and hFGF23 comprise the following residues in hFGF23:
Chem. 206: R76, T86, R91, Y93, Y107, R114, F169 and N170.
Chem. 207: W36, R76, T86, R91, Y107, R114, 1167, F169 and N170.
Chem. 208: R76, T86, R91, R114, L166 and F169.

Taken together with Example 8 all tested compounds bind to an epitope comprising amino acid residues R76, T86, R91 and R114 on hFGF23. These residues (highlighted in bold above) are thus regarded as key epitope residues for the compounds of the invention.

### Example 10: Pharmacokinetic Study 2 in Minipigs

The aim of the study was to determine the half-life in blood following i.v. administration of certain compounds of the invention.

The studies were done in female Göttingen minipigs (Ellegaard Minipigs A/S, Dalmose, Denmark) with a body weight of approximately 25 kg. The polypeptides were administered to the minipigs intravenously, n=3 per group, and the dose was 14 nmol/kg. The vehicle of the formulations was 50 mM phosphate and 70 mM sodium chloride and 0.007% polysorbate20 at pH 7.4.

Blood was sampled for up to 312 hours through an indwelling permanent venous catheter and collected in EDTA tubes (Sarstedt, Germany). Plasma was separated and analysed for compound concentration using Liquid chromatography mass spectrometry (LC-MS).

Non-compartmental (NCA) pharmacokinetic analysis was done in PHOENIX© WINNONLIN® v. 8.1 (Certara L. P. Princeton, NJ, USA). The terminal elimination phase was fitted via linear regression with uniform weighting. Nominal sampling times and actual doses were used for NCA.

Concentrations of polypeptides in minipig plasma were assessed with plasma protein precipitation followed by turboflow liquid chromatography mass spectrometry (TF-LC-MS). Calibration standards and quality control samples were prepared by spiking compounds into blank plasma followed by serial dilution. All plasma samples underwent sample preparation by addition of four volumes of ethanol to one volume of plasma sample. The precipitation solvent contained 20 nM internal standard. Following centrifugation at 4° C., the supernatant was diluted with one volume of purified water containing 1% formic acid. Precipitated plasma samples were first cleaned up and concentrated on a TurboFlow Cyclone column (0.5×50 mm from Thermo Fisher Scientific) operated at 30° C. and then transferred to an analytical column. The analytical column was an XBridge Peptide BEH C18 column (2.1×50 mm, 3.5 μm particle size from Waters) and operated at 60° C. Analytical separations were conducted with a linear gradient. Mobile phase A was water with 5% organic solvent (acetonitrile/methanol (1:1, by volume)) and 1% formic acid, and mobile phase B was water with 95% organic solvent (acetonitrile/methanol (1:1, by volume)) and 1% formic acid. The mass spectrometer coupled to the TF-LC system was either a TSQ Altis or a Q Exactive (both from Thermo Fisher Scientific). The mass spectrometers were operated in positive ionization mode and selected reaction monitoring on the TSQ Altis and parallel reaction monitoring on the Q Exactive. Samples were quantified using linear regression against the response from the calibration standards with 1/X² weighting. The response was the analyte peak area divided by the internal standard peak area. Measured concentration for quality control samples and calibration standards were within ±15% of the nominal concentration, except for one quality control sample that was −17% of nominal concentration. The lower limit of quantification was between 0.250 nM and 1.00 nM.

### Results

Results are shown in Table 13 below, wherein column T½ shows the half-life (T½) in blood. A high half-life value is desirable.

TABLE 13
Pharmacokinetic parameters after intravenous administration

| Chem. | T½ (h) | Protractor |
|---|---|---|
| 206 | 61.5 | C20 diacid |

No adverse clinical observations deemed related to the test compound were observed during the study.

The result show that protraction at position 58 with the C20 diacid results in clinically meaningful half-life in blood.

A long half-life indicates that the given compound is capable of providing its inhibitory effect for a prolonged period after dosing in comparison with a compound having a relatively shorter half-life. Longer half-life permits less frequent dosing when in use, which is desirable to provide an increased convenience for the patients.

### Example 11: Plasma Phosphate Levels in Minipigs

In order to determine the in vivo effect of the compound of the invention designated Chem. 206 herein, plasma phosphate levels were measured in the PK study described in Example 10.

### Method Serum Pi:

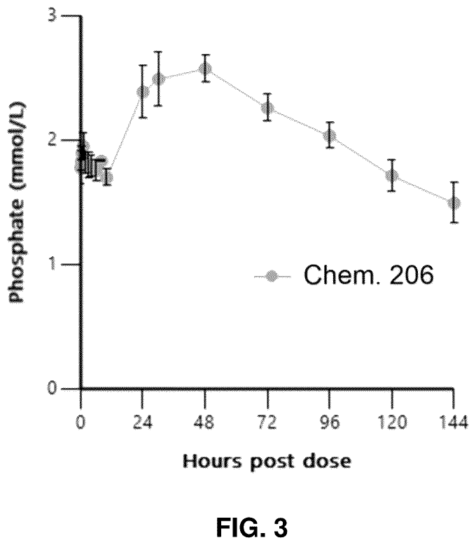

Plasma phosphate was measured with a PHOS2 inorganic phosphate assay v2.0 (Roche) on a Cobas pro c503 analytical instrument. Animals were administered a single intravenous dose of 14 nmol/kg of compound. Plasma samples for analysis were collected at dosing (0), at 5, 15 and 30 minutes, and at 1, 1.5, 2, 3, 4, 6, 8, 10, 24, 30, 48, 72, 96 and 144 hours post dose. Data is reported in FIG. 3/Table 14 showing plasma phosphate levels in mmol/L.

TABLE 14
Plasma phosphate levels (mmol/L) in
minipigs after intravenous dosing

| | |
|---|---|
| Time | Chem. 206 |

| | | |
|---|---|---|
| 0 | min | 1.78 |
| 5 | min | |
| 15 | min | 1.83 |
| 30 | min | 1.90 |
| 1 | h | 1.95 |
| 1.5 | h | 1.82 |
| 2 | h | 1.80 |
| 3 | h | 1.80 |
| 4 | h | 1.76 |
| 6 | h | 1.83 |
| 8 | h | 1.70 |
| 10 | h | 2.39 |
| 24 | h | 2.49 |
| 30 | h | 2.58 |
| 48 | h | 2.26 |
| 72 | h | 2.04 |
| 96 | h | 1.72 |
| 144 | h | 1.50 |

The results show that compounds of the invention are capable of increasing plasma phosphate levels in vivo.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A hFGF23 antagonistic polypeptide, which is capable of binding an epitope comprising the amino acid residues selected from the group consisting of:
a) R76, T86, R91 and R114;
b) M74, R76, T86, R91, F108, P110, R114, L166, I167 and N170;
c) R76, T86, R91, Y93, Y107, R114, F169 and N170;
d) W36, R76, T86, R91, Y107, R114, 1167, F169 and N170; and
e) R76, T86, R91, R114, L166 and F169;
on hFGF23 according to SEQ ID NO:1 as determined at 3.5 Å.

2. A hFGF23 antagonistic polypeptide, which is capable of binding hFGF23 according to SEQ ID NO:1, comprising a binding motif (BM) which consists of an amino acid residue sequence selected from
i)
XₐX_{b}X_{c}WX_{d}EIXₑX_{f}X_{g}PXₕLXᵢDXⱼQWPAFIEXₖLH
according to SEQ ID NO: 2
wherein, independently of each other,
Xₐ is F or Y;
X_{b} is F, I, L or V;
X_{c} is A or Q;
X_{d} is A, F, H, Y or W;
Xₑ is F or Y;
X_{f} is N, Q or T;
X_{g} is A or L;
Xₕ is C, H, N, Q, W or Y;
Xᵢ is D, N, Q, S or T;
Xⱼ is A, D, E, G, H, L, N, Q, S, T or Y;
Xₖ is A, Q or S; and
ii) an amino acid residue sequence having at least 93% identity to the sequence defined in i).

3. The hFGF23 antagonistic polypeptide according to claim 2, wherein the (BM) is selected from the group consisting of:
SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51 and SEQ ID NO:52;
optionally comprising 1, 2 or 3 substitution(s), wherein said substitution(s) can take place in any one of positions 1, 2, 3, 6, 9, 10, 11, 13, 15, 17 and/or 25 in the selected (BM) sequence.

4. The hFGF23 antagonistic polypeptide according to claim 3, wherein the substitution(s) is/are conservative substitution(s).

5. The hFGF23 antagonistic polypeptide according to claim 2, which comprises a binding motif (BM), wherein said polypeptide comprises an amino acid residue sequence selected from
a)
X₁X₂X₃X₄X₅X₆X₇X₈-(BM)-X₃₆X₃₇PSQX₄₁X₄₂X₄₃
LLX₄₆EARX₅₀LX₅₂X₅₃X₅₄QX₅₆X₅₇X₅₈
according to SEQ ID NO: 3,
wherein (BM) consists of an amino acid residue sequence as defined in claim 2(i),
wherein, independently of each other,
X₁ is G, I, L, N or V;
X₂ is D, E or Q;
X₃ is D, E, G, N or Q;
X₄ is D, E, H, N, R or T;
X₅ is E, I, L, T or V;
X₆ is F, W or Y;
X₇ is A, G, H, I, L, N, P, Q, R, T or V;
X₈ is A, L or E;
X₃₆ is A, D or E;
X₃₇ is D or E;
X₄₁ is A, R or W;
X₄₂ is A, D or T;
X₄₃ is E, N, Q or S;
X₄₆ is A, E or K;
X₅₀ is Q or R;
X₅₂ is N or E;
X₅₃ is D, E, K or T;
X₅₄ is A or I;
X₅₆ is A or C;
X₅₇ is K or P;
X₅₈ is C, K or E; and
b) an amino acid residue sequence having at least 93% identity to the sequence defined in a).

6. The hFGF23 antagonistic polypeptide according to claim 5 wherein, independently of each other,
X₁ is G, I, L or V;
X₂ is D or E;
X₃ is D or E;
X₄ is D or E;
X₅ is E;
X₆ is F;
X₇ is I or Q;
X₈ is A or L;
Xₐ is Y;
X_{b} is V;
X_{c} is Q;
X_{d} is Y;
Xₑ is F or Y;
X_{f} is N or Q;
X_{g} is L;
Xₕ is N;
Xᵢ is N or T;
Xⱼ is A, N or Q;
Xₖ is S;
X₃₆ is D or E;
X₃₇ is D or E;
X₄₁ is W;
X₄₂ is A;
X₄₃ is N;
X₄₆ is A or E;
X₅₀ is Q or R;
X₅₂ is N;
X₅₃ is E;
X₅₄ is A;
X₅₆ is A;
X₅₇ is P;
X₅₈ is K.

7. The hFGF23 antagonistic polypeptide according to claim 2, wherein said binding motif (BM) forms part of a three-helix bundle protein domain, said three-helix bundle protein domain being a variant of protein Z, which is derived from domain B of staphylococcal Protein A.

8. An hFGF23 antagonistic polypeptide, which is capable of binding hFGF23 according to SEQ ID NO:1, having at least 93% identity to a hFGF23 antagonistic polypeptide selected from the group consisting of:
Chem. 197 according to SEQ ID NO:168;
Chem. 201 according to SEQ ID NO:176;
Chem. 204 according to SEQ ID NO:234;
Chem. 206 according to SEQ ID NO:236;
Chem. 207 according to SEQ ID NO:237; and
Chem. 208 according to SEQ ID NO:238.

9. The hFGF23 antagonistic polypeptide according to claim 2, wherein the polypeptide is selected from the group consisting of:
Chem. 89;
Chem. 106;
Chem. 107;
Chem. 111;
Chem. 197;
Chem. 201;
Chem. 204;
Chem. 206;
Chem. 207; and
Chem. 208.

10. The hFGF23 antagonistic polypeptide according to claim 2 wherein the polypeptide is capable of binding hFGF23 with a K_{D} value of less than 100 μM such as less than 10 μM, such as less than 9 μM, such as less than 8 μM, such as less than 7 μM, such as less than 6 μM, such as less than 5 μM, such as less than 4 μM, such as less than 3 μM, such as less than 2 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 10 nM, such as less than 1 nM, preferably less than 0.1 μM, such as less than 10 nM, such as less than 2 nM, such as less than 1 nM.

11. The hFGF23 antagonistic polypeptide according to claim 2 wherein said polypeptide comprises a protraction moiety.

12. The hFGF23 antagonistic polypeptide according to claim 11 wherein the protraction moiety is covalently attached to an amino acid residue side chain group on said polypeptide.

13. The hFGF23 antagonistic polypeptide according to claim 11 wherein the protraction moiety comprises a C16, C17, C18, C19, C20, C21 or C22 fatty acid, such as a C18, C19 or C20 fatty diacid.

14. The hFGF23 antagonistic polypeptide according to claim 11 wherein said protraction moiety comprises a protractor “P” selected from the group consisting of: 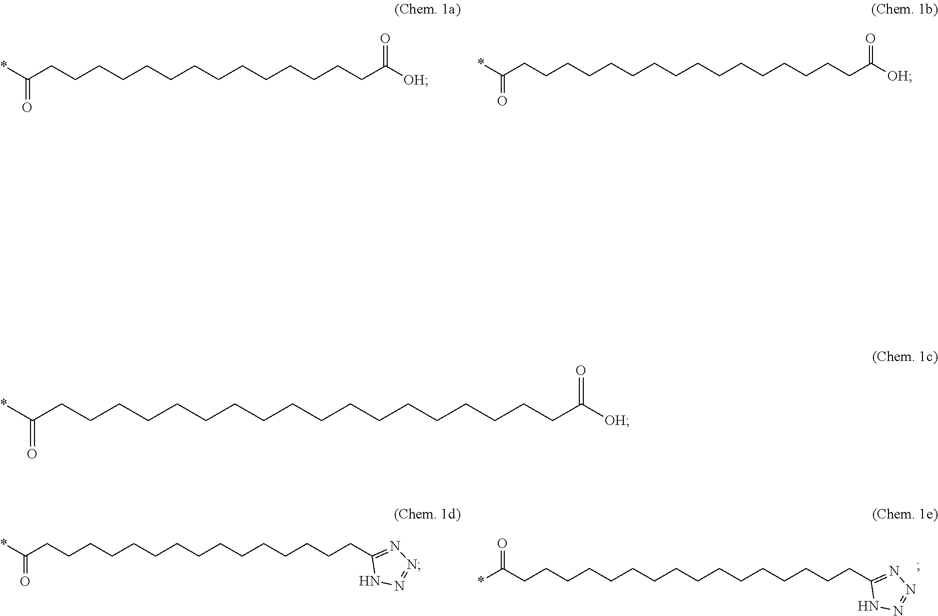 [SMILES: *C(=O)CCCCCCCCCCCCCCC(=O)O ; *C(=O)CCCCCCCCCCCCCCCCC(=O)O ; *C(=O)CCCCCCCCCCCCCCCCCCC(=O)O ; *C(=O)CCCCCCCCCCCCCCCc1nnn[nH]1 ; *C(=O)CCCCCCCCCCCCCCCCc1nnn[nH]1]
(Chem. 1f, SEQ ID NO: 9)
QRLMEDICLPRWGCLWEDDF -*;
and
(Chem. 1f, SEQ ID NO: 9)
*-QRLMEDICLPRWGCLWEDDF;
wherein * represents the attachment point to either a linker (L_{P}) or the hFGF23 antagonistic polypeptide.

15. The hFGF23 antagonistic polypeptide according to claim 2 wherein the polypeptide is a polypeptide derivative comprising a protraction moiety wherein said protraction moiety comprises a linker “L_{P}” selected from the group consisting of:
*—NH—(CH₂)₂—(O—(CH₂))ₖ—O—(CH₂)ₙ—CO—* (chem. 2a), wherein k is an integer in the range of 1-5, and n is an integer in the range of 1-5;

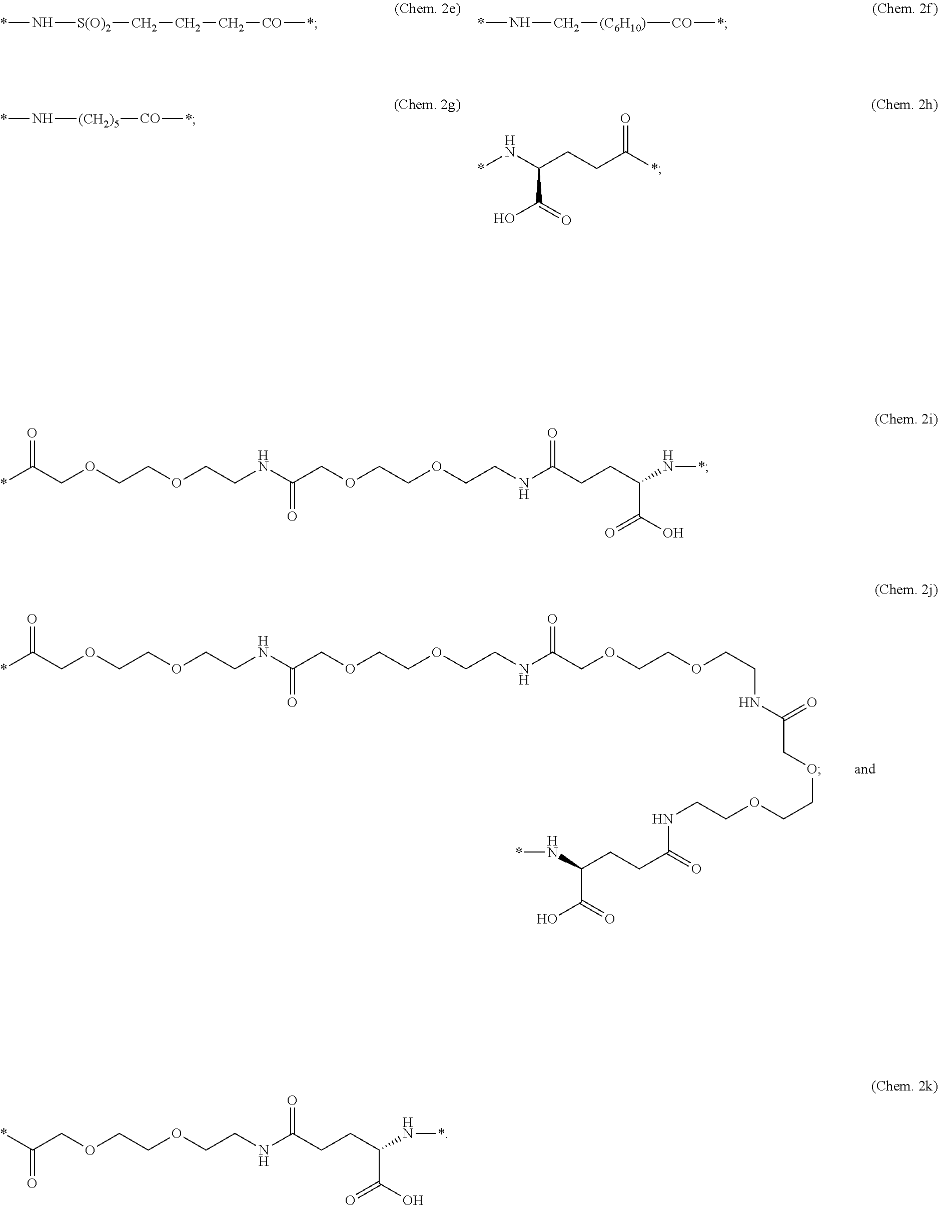

16. The hFGF23 antagonistic polypeptide according claim 15 wherein the protractor “P” is Chem. 1c and the linker L_{P} is Chem. 2i (L1).

17. The hFGF23 antagonistic polypeptide according claim 2 wherein the polypeptide is a polypeptide derivative comprising a protraction moiety wherein said protraction moiety is covalently attached to the side chain of a cysteine or a lysine residue in said polypeptide.

18. A compound selected from the group consisting of:

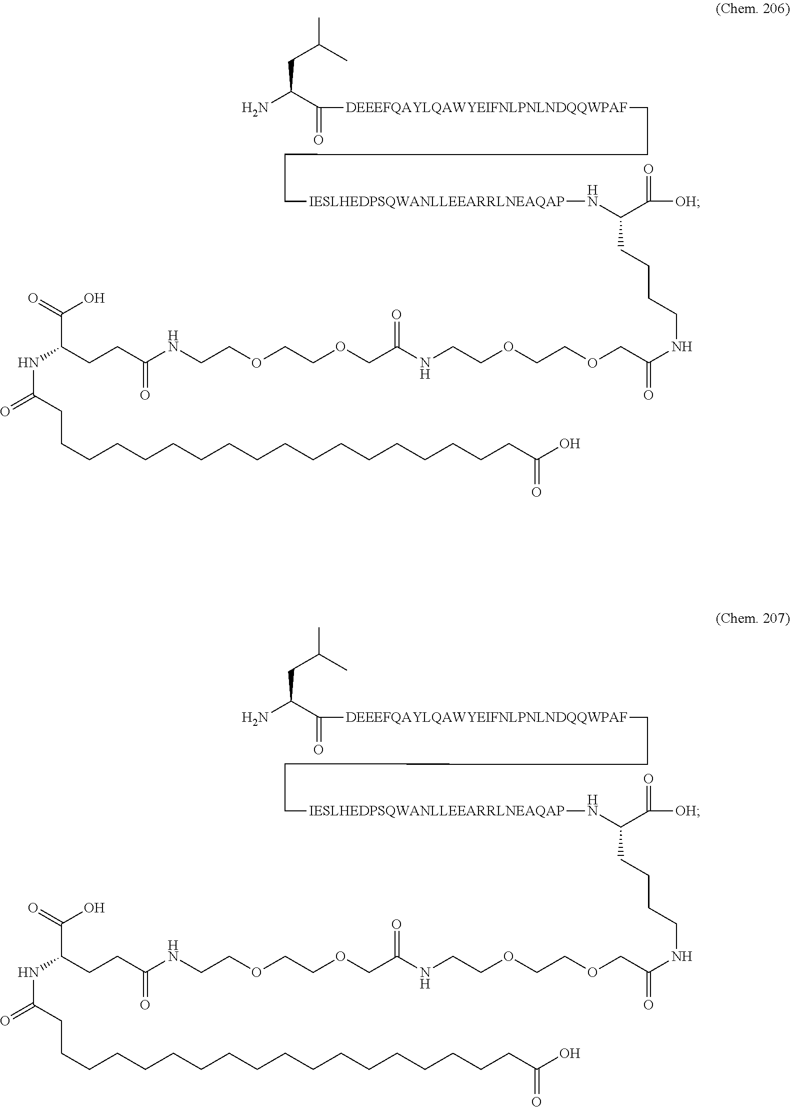

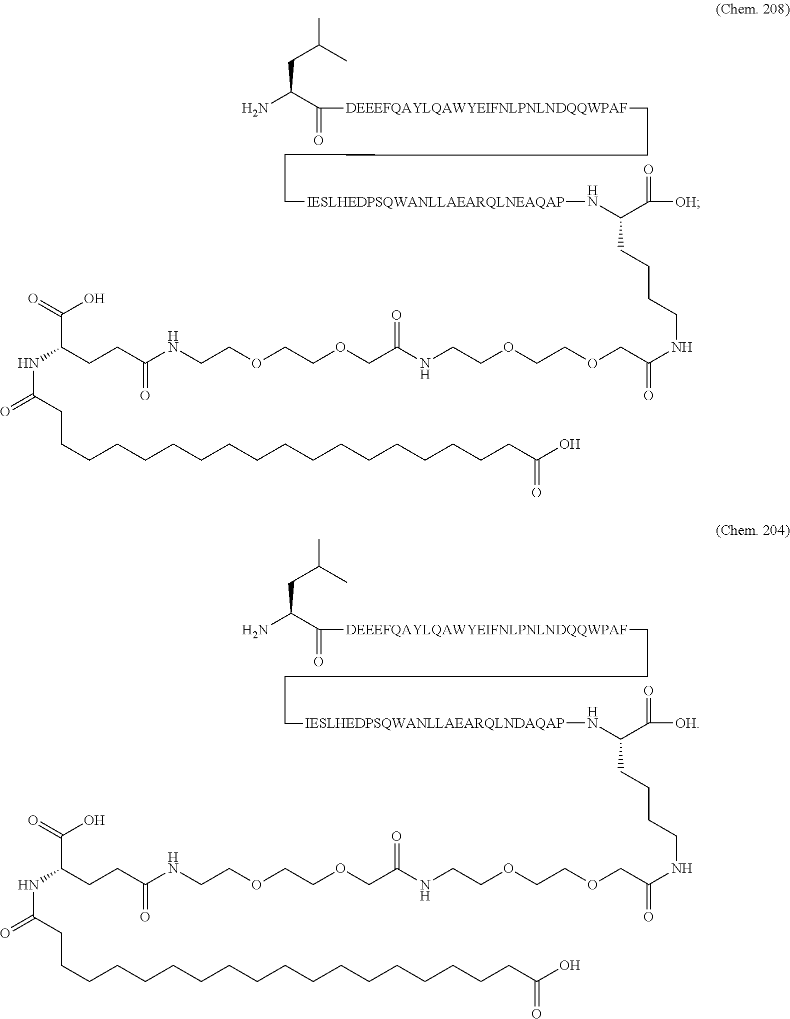

19. The compound according to claim 18 wherein the compound is a hFGF23 antagonistic polypeptide derivative capable of binding hFGF23 according to SEQ ID NO:1.

20. A hFGF23 antagonistic polypeptide derivative according to claim 18 represented by the formula:

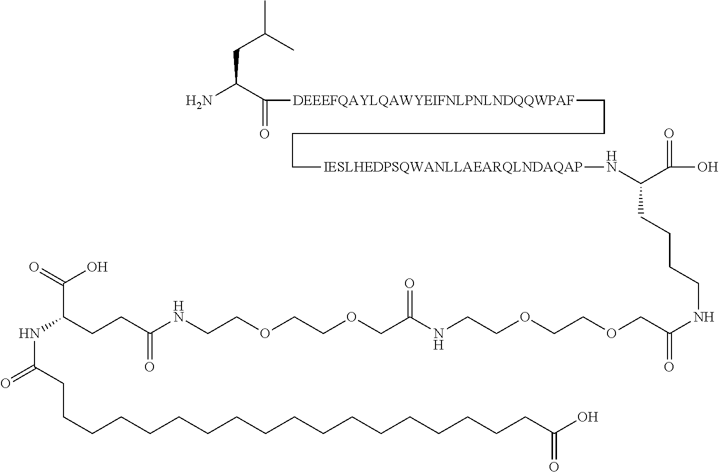

21. A pharmaceutical composition comprising the hFGF23 antagonistic polypeptide, polypeptide derivative or compound according to claim 2 and one or more pharmaceutically acceptable excipient(s).

22. (canceled)

23. A method of treating tumor-induced osteomalacia, fibrous dysplasia, McCune-Albright syndrome, autosomal dominant hypophosphatemic rickets (ADHR) or X-linked hypophosphatemic rickets (XLH) comprising administering an effective amount of the hFGF23 antagonistic polypeptide, polypeptide derivative, compound, or composition according to to a patient in need thereof.

24. A kit comprising the hFGF23 antagonistic polypeptide, polypeptide derivative, compound, or composition according to claim 2 and instructions for use.
